(19) United States
(12) Patent Application Publication
(10) Pub. No.: US 2023/0357834 A1
(43) Pub. Date: Nov. 9, 2023

(54) HYBRID PROTOCOLS AND BARCODING SCHEMES FOR MULTIPLE SEQUENCING TECHNOLOGIES

(71) Applicant: The Regents of the University of California, Oakland, CA (US)

(72) Inventors: Charles Chiu, Oakland, CA (US); Wei Gu, Oakland, CA (US); Xianding Deng, Oakland, CA (US); Shaun Arevalo, Oakland, CA (US); Allan Gopez, Oakland, CA (US)

(21) Appl. No.: 18/026,287

(22) PCT Filed: Sep. 24, 2021
(86) PCT No.: PCT/US2021/051924

§ 371 Date: Mar. 14, 2023

Related U.S. Application Data
(60) Provisional application No. 63/083,868, filed on Sep. 26, 2020.

(51) Int. Cl. C12Q 1/6869

(52) U.S. Cl. CPC C12Q 1/6869

(57) ABSTRACT

The disclosure provides for hybrid protocols and barcoding schemes that allow for sequencing of targeted polynucleotides in multiple types of sequencing platforms, and applications thereof, including for metagenomic analysis.

### CROSS REFERENCE TO RELATED APPLICATIONS

This application is a U.S. National Phase Application filed under 35 U.S.C. § 317 and claims priority to International Application No. PCT/US2021/051924, filed Sep. 24, 2021, which application claims priority under 35 U.S.C. § 119 from Provisional Application Ser. No. 63/083,868 filed Sep. 26, 2020, the disclosures of which are incorporated herein by reference.

### STATEMENT OF GOVERNMENT SUPPORT

This invention was made with government support under grants HL105704, R21 AI120977 and R33 AI129077, awarded by The National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Oct. 28, 2021, is named 00138-012WO1_SL.txt and is 114,277 bytes in size.

### TECHNICAL FIELD

The disclosure provides for hybrid protocols and barcoding schemes that allow for sequencing of targeted polynucleotides in multiple types of sequencing platforms, and applications thereof, including for metagenomic analysis.

### BACKGROUND

Early detection of causative microorganisms in patients with severe infections is important to informing clinical interventions and administering appropriately targeted antibiotics. Timely and accurate diagnosis, however, remains highly challenging for many hospitalized patients. As most infectious syndromes present with indistinguishable clinical manifestations, broad-based, multiplexed diagnostic tests are urgently needed but not yet available for the vast majority of potential pathogens. Some microorganisms are difficult to grow in culture (e.g., Treponema pallidum, Bartonella sp.), or unculturable (e.g., some viruses), while others (e.g., mycobacteria and molds) can take weeks to grow and speciate. Accurate molecular detection by PCR provides an alternative diagnostic approach to culture, but is hypothesis-driven and thus requires a priori suspicion of the causative pathogen(s).

### SUMMARY

Metagenomic analysis by next-generation sequencing of random, “shotgun” reads has a number of applications, including (1) clinical diagnosis, (2) pathogen discovery, (3) de novo genome assembly, (4) whole-exome sequencing, (5) targeted gene panel sequencing, (5) transcriptome profiling, and (6) whole-genome resequencing. Disclosed herein is a metagenomic next-generation sequencing (mNGS) method using cell-free DNA from body fluids to identify pathogens. The performance of mNGS testing of 182 body fluids from 160 acutely ill patients was evaluated using two sequencing platforms in comparison to microbiological testing using culture, 16S bacterial PCR, and/or 28S-ITS fungal PCR. Test sensitivity and specificity of detection were 79% and 91% for bacteria and 91% and 89% for fungi, respectively, by Illumina sequencing; 75% and 81% for bacteria and 91% and 100% for fungi, respectively, by nanopore sequencing. In a case series of 12 patients with culture/PCR-35 negative body fluids but for whom an infectious diagnosis was ultimately established, 7 (58%) were mNGS-positive. Real-time computational analysis enabled pathogen identification by nanopore sequencing in a median 50-minute sequencing and 6-hour sample-to-answer time. The Rapid mNGS methods of the disclosure are promising tools for diagnosis of unknown infections from body fluids.

The disclosure provides an oligonucleotide comprising barcodes for use in multiple types of next generation sequencing technologies, the barcodes comprising at least about 18 to about 160 nucleotides in length having a first nucleotide domain and at least one second nucleotide domain; wherein the first nucleotide domain comprises 4-12 nucleotides (4-12mer) of the barcode located at either end of the barcode and wherein the 4-12mer are compatible with a next generation sequencing technology that utilizes bridge amplification, wherein the second nucleotide domain comprises 14-35 nucleotides (14-35mer) of the barcode and wherein the 14-35mers are compatible with a next generation sequencing that utilizes nanopores, wherein at least a minimum Levenshtein distance between a pair of 4-12mers is utilized, and wherein the Levenshtein distance has been maximized between a pair of barcodes in order to minimize barcode “crosstalk”. In one embodiment, the oligonucleotide further comprises a flow cell attachment domain. In a further embodiment, the flow cell attachment domain comprises a sequence selected from SEQ ID NO:1, 2, 3 or 4. In another embodiment, the oligonucleotide further comprises a sequencing primer binding domain. In another embodiment, the barcode is comprised of the 4-12mer and the second domain comprises 3 sets of 10mers that when concatenated together form a 34-42mer, wherein the last nucleotide is removed to form the 33-41mer barcode. In another embodiment, the oligonucleotide comprises a sequence selected from any one of SEQ ID Nos: 226-416 and 417. In another embodiment of any of the foregoing embodiments, oligonucleotide consists of 47-80 nucleotides. In another embodiment, the oligonucleotide is 62-83 nucleotides in length.

The disclosure also provides an oligonucleotide comprising barcodes for use in multiple types of next generation sequencing technologies, the barcodes comprising at least about 18 to about 39 nucleotides in length having a first nucleotide domain and at least one second nucleotide domain; wherein the first nucleotide domain comprises 4-9 nucleotides (4-9mer) of the barcode located at either end of the barcode and wherein the 4-9mers are compatible with a next generation sequencing technology that utilizes bridge amplification, wherein the second nucleotide domain comprises 14-35 nucleotides (14-35mer) of the barcode and wherein the 14-35mers are compatible with a next generation sequencing that utilizes nanopores, wherein at least a minimum Levenshtein distance between a pair of 4-9mers is utilized, and wherein the Levenshtein distance has been maximized between a pair of barcodes in order to minimize barcode “crosstalk”.

The disclosure also provides an oligonucleotide barcode sequence for use in multiple types of next generation sequencing, wherein the oligonucleotide barcode is about 24 to 39 nucleotides in length and comprises a first oligonucleotide barcode domain of about 4-12 nucleotides (4-12mer) at the 5′ or 3′ end of the oligonucleotide barcode and a second oligonucleotide barcode domain of about 10-29 nucleotides in length operably linked to the first oligonucleotide barcode domain, wherein the Levenshtein distance has been maximized between a pair of oligonucleotide barcodes in order to minimize barcode “crosstalk”; wherein the first oligonucleotide barcode domain is compatible with next generation sequencing using bridge amplification; wherein the second oligonucleotide barcode domain is compatible with next generation sequencing using nanopores; and wherein the oligonucleotide has a minimum Levenshtein distance between a pair of 4-9mers. In one embodiment, the barcode is about 36-39 nucleotides in length. In still another or further embodiment, the oligonucleotide comprises a sequence selected from the group consisting of SEQ ID Nos: 226-416 and 417.

The disclosure also provides a set of oligonucleotides comprising a barcode as set forth herein. In another embodiment, each barcode is located between a pair of sequencing adaptors. In still a further embodiment, the pair of sequencing adaptors have sequences selected from (i) or (ii): (i) CAAGCAGAAGACGGCATACGAGAT (SEQ ID NO:1), and GTGACTGGAGTTCAGACGTGTGCTCTTCCGATC*T (SEQ ID NO:2); or (ii) AATGATACGGCGACCACCGAGATCTACAC (SEQ ID NO:3), and ACACTCTTTCCCTACACGACGCTCTTCCGATC*T (SEQ ID NO:4), wherein * indicates a phosphorothioate bond between the nucleotides. In still another embodiment, the set of oligonucleotides are PCR primers used for sequencing library barcoding.

The disclosure also provides a sequencing library comprising the set of barcodes as described herein. In another embodiment, the sequencing library is used for an application selected from: pathogen discovery, environmental metagenomics, de novo genome assembly, whole-exome sequencing, transcriptomics sequencing, targeted gene panel sequencing or whole-genome resequencing.

The disclosure also provides a method for rapid pathogen detection in a sample using metagenomic next-generation sequencing (mNGS), comprising: obtaining one or more samples comprising cell-free DNA (cfDNA); generating a plurality of sequencing reads comprising a barcode from the set of barcodes of the disclosure using next-generation sequencing; performing metagenomic analysis on the plurality of sequencing read data using a clinical bioinformatics software pipeline that can rapidly analyze sequencing read data for pathogenic DNA; determining and identifying pathogen(s) in the one or more samples based upon the metagenomic analysis of the sequencing read data. In another embodiment, the one or more samples comprises a body fluid sample from a subject. In a further embodiment, the body fluid sample is an infected body fluid sample. In still another or further embodiment, the body fluid sample is selected from cerebrospinal fluid, urine, semen, pericardial fluid, pleural fluid, peritoneal fluid, synovial fluid, amniotic fluid, fetal fibronectin, saliva, sweat, eye vitreous humor, eye aqueous humor, bronchoalveolar lavage fluid, breast milk, bile, and ascites fluid. In still a further embodiment, the one or more samples further comprise a blood serum sample. In another embodiment, the next-generation sequencing comprises sequencing technology that utilizes bridge amplification. In another or further embodiment, the next-generation sequencing comprises or further comprise sequencing technology that utilizes nanopores. In still another embodiment, the clinical bioinformatics software pipeline that can rapidly analyze sequencing read data for pathogenic DNA is SURPI+ or SURPIrt. In still another embodiment, the pathogen(s) comprise one or more pathogenic bacteria. In another embodiment, the pathogen(s) comprise one or more pathogenic fungi.

The disclosure provides a set of paired 37mer barcodes comprising dual indexes that are configured for dual use in multiple types of next generation sequencing technologies, wherein the Levenshtein distance has been maximized between each pair of 37mer barcodes in order to minimize barcode “crosstalk”; wherein the first 8 nucleotides (8mer) of each pair of 37mer barcodes is compatible with a next generation sequencing technology that utilizes bridge amplification, and wherein at least a minimum Levenshtein distance between each pair of 8mers is utilized; wherein at least a minimum Levenshtein distance between each pair of 37mers barcodes is used so that the 37mer barcode is compatible with a next generation sequencing technology that utilizes nanopores.

In a certain embodiment, the disclosure provides for a composition or method as substantially described and/or illustrated herein.

### DESCRIPTION OF DRAWINGS

FIG. 1-C provides various embodiments of the metagenomic next-generation sequencing (mNGS) method of the disclosure. (A) Schematic of mNGS body fluid analysis workflow. The clinical gold standard consisted of aggregated results from cultures, bacterial 16S PCR, and/or fungal 28S-ITS PCR, while the composite standard also included confirmatory digital PCR with Sanger sequencing and clinical adjudication. For nanopore sequencing in <6 h, 40-60 min are needed for nucleic acid extraction, 2-2.5 h for mNGS library preparation, 1 h for nanopore 1D library preparation, and 1 h for nanopore sequencing and analysis. (B) Analysis workflow for the 182 total body fluid samples in the study. 170 samples were included in the accuracy assessment, while 12 samples collected from patients with a clinical diagnosis of infection but negative microbiological testing were included for mNGS analysis. The pie chart displays the body fluid sample types analyzed in the study. (C) Timing for mNGS testing relative to culture. Whereas culture-based pathogen identification can take days to weeks, mNGS testing using nanopore or Illumina sequencing platforms has a 5-24 h overall turnaround time.

FIG. 2A-F demonstrates mNGS testing accuracy and relative pathogen burden in body fluid samples. (A) ROC curves of Illumina (n=43 samples) and nanopore (n=42 samples) training sets based on culture and 16S testing. Plotted are mNGS test sensitivities and specificities at nRPM threshold values ranging from 0.1 to 100. (B) ROC curves of both training sets based on a composite standard. (C) Contingency tables for the independent Illumina (n=127 samples) and nanopore (n=43 samples) validation sets. PPA and NPA are shown in lieu of sensitivity and specificity respectively if a composite standard was used. The scoring system for determination of positive and negative results is described in Table 5. (D) ROC curves stratified by body fluid type (n=170 samples in total). Plotted is the performance of the combined Illumina training and validation datasets relative to composite standard testing. Plasma is not counted as a body fluid in panels D and F but is plotted as a separate set. (E) Direct comparison of Illumina with nanopore sequencing (79 bacteria) across all body fluids. The yield of pathogen-specific reads based on a nRPM metric is linearly correlated and comparable between nanopore and Illumina sequencing. (F) Relative pathogen burden in body fluids, stratified by body fluid and microorganism type. The burden of pathogen cfDNA in body fluid samples is estimated using calculated nRPM values. Based on Illumina data, bacterial cfDNA in plasma was significantly lower on average than in local body fluids (p=0.0035), and pathogen cfDNA in body fluids was significantly higher for bacteria than for fungi (p=0.0049). All box plots represent the median (centre), the interquartiles (minima and maxima), and 1.5× interquartile range (whiskers). All p-values are calculated using a two-sided Welch's t-test. Abbreviations: ROC, receiver operator characteristic; nRPM, normalized reads per million; PPA, positive percent agreement; NPA, negative percent agreement; cfDNA, cell-free DNA.

FIG. 3A-C provides a comparison of mNGS with 16S (bacterial) or 28S-ITS (fungal) PCR. The Venn diagram shows all cases out of 182 where mNGS and associated 16S or 28S-ITS PCR detected a microorganism. Krona plots depict genus and species levels of all sequence-matched bacterial or fungal reads depending on the microorganism type. (A) mNGS and 16S/28S-ITS PCR testing results for 14 culture-negative body fluid samples. (B) Concordant bacterial cases (n=7 samples). (C) Discordant bacterial cases (n=3 samples). In case S31 (top left), mNGS identified the causative pathogen in a case of necrotizing pneumonia, Klebsiella pneumoniae, whereas 16S PCR testing was falsely positive for Streptococcus mitis. In case S88 (right), mNGS identified Klebsiella aerogenes in CSF that tested negative by culture and 16S PCR, a finding confirmed by culture of an infected deep brain stimulator located upstream of the lumbar puncture site (drawing and axial slice). Nanopore sequencing was able to detect both bacteria within 3 minutes after start of sequencing (xy plots with dotted line showing the detection threshold). In case S144 (bottom left), Mycobacterium avium complex was detected by 16S PCR but not by mNGS. (D) Fungal cases (n=4 samples). In 3 discordant cases, mNGS testing detected the causative pathogen while 28S-ITS testing was negative. All 3 mNGS results was orthogonally confirmed by concurrent or subsequent culture of the body fluid or culture of biopsy tissue. For additional details on the cases, please see Table 10 and Clinical Vignettes presented in the Examples. Abbreviations: BAL, bronchoalveolar lavage fluid; CT, computed tomography.

FIG. 4A-B provides a comparison of relative pathogen burden in paired body fluid and plasma samples. (A) Schematic showing concurrent collection of blood plasma and body fluid samples from the same patient. (B) Bar plot of the nRPM corresponding to 9 organisms in paired body fluid and plasma samples from 7 patients. The vertical lines show the thresholds used for a positive bacterial (nRPM=2.6) or fungal (nRPM=0.1) detection. The checkboxes denote microorganisms that were not identified by conventional microbiological testing (culture and/or 16S PCR) but that were orthogonally confirmed by dPCR, serology, and/or clinical adjudication (see Clinical Vignettes presented in the Examples). Abbreviation: nRPM, normalized reads per million.

FIG. 5A-I presents metagenomic sequencing of body fluids. (A) Log scale plot of the bacterium Achromobacter xylosoxidans from mNGS data, a common background contaminant in sequencing libraries. There is a log-linear relationship between the qPCR cycle threshold (Ct) value and the RPM corresponding to Achromobacter xylosoxidans. The background level of Achromobacter xylosoxidans is inversely correlated with the input concentration and is relatively constant. (B) Precision-recall curves based on the Illumina training bacterial dataset in comparison with the composite standard. (C) Precision-recall curves based on nanopore bacterial training datasets. (D) Precision-recall curves for Illumina and nanopore training fungal datasets. (E) Pie chart showing distribution of bacterial pathogen titers as estimated by semi-quantitative culture. (F) Plot of nRPM values versus semi-quantitative bacterial titers. The nRPM corresponding to bacteria cultured in enrichment broth was significantly lower than the other higher-titer cultures (p=0.006). (G) Relative pathogen burden in positive and negative (non-infectious) body fluid samples. (H) Nanopore time to detection (minutes) across different body fluid types. Each data point represents the time to detection of the organism, if any, in each body fluid sample. (I) Nanopore time to detection (minutes) in relation to pathogen DNA abundance in samples (reads per million, RPM). All box plots represent the median (centre), the interquartiles (minima and maxima), and 1.5× interquartile range (whiskers).

FIG. 6A-D presents ROC curves of mNGS test performance. ROC curves are plotted from validation set data based on a clinical gold standard or composite standard. Data are presented as median true positive rates +/− the 95% confidence intervals. The 95% confidence interval was obtained via a bootstrap method with 2000 resampling iterations. (A) Illumina dataset, bacterial detection (n=127 samples). (B) Nanopore dataset, bacterial detection (n=43 samples), (C) Illumina dataset, fungal detection (n=127, 32 fungal organisms). (D) Nanopore dataset, fungal detection (n=43, 11 fungal organisms).

FIG. 7A-D displays the relationship of external positive control organism titer with mNGS detection signal (expressed in nRPM). Simple linear regression of normalized reads per million (nRPM) over four replicates per dilution factor, calculated as genome equivalents per mL (GE/mL) for (A) Streptococcus uberis, (B) Rhodobacter sphaeroides, (C) Millerozyma farinosa, and (D) Aspergillus oryzae.

FIG. 8A-E provides orthogonal testing for Case S31: Klebsiella pneumoniae infection of pleural fluid. (A) Genomic coverage of K. pneumoniae from Illumina mNGS. Sequencing spanned 36,490 base pairs, or 0.65% of the K. pneumoniae genome. (B) Orthogonal confirmation of K. pneumoniae by dPCR of the sequencing library. Nine negative controls from other cases were run in parallel. Out of 10 sequencing libraries, only Case S31 had any positive droplets (n=43 of 12022 total droplets as circled). (C) Orthogonal confirmation of K. pneumoniae by dPCR of the DNA extract. Three positive droplets were detected, indicating a low positive result. (D) Orthogonal confirmation of K. pneumoniae by dPCR of contralateral pleural fluid (sample C31). 29 and 24 positive droplets were detected out of 2 replicates. Digital PCR targeting Streptococcus mitis on both pleural fluids did not yield any positive droplets. The positive controls for these experiments were from sheared DNA from Klebsiella pneumoniae and Streptococcus mitis respectively, whereas the negative control was water. (E) Sanger sequencing of the K. pneumoniae amplicon from dPCR. Shown are sequencing traces confirming the presence of K. pneumoniae (SEQ ID NOS 419, 418, 418, 418, and 418, respectively, in order of appearance).

FIG. 9A-C provides orthogonal testing for Cases S88: Klebsiella aerogenes from cerebrospinal fluid and S87: Bartonella henselae from a skin abscess. (A) Orthogonal confirmation of K. aerogenes by dPCR of the DNA extract. The sample was run in parallel with 9 negative controls. Out of 10 sequencing libraries, only Case S88 had positive dPCR droplets (n=61). (B) Genomic coverage of K. aerogenes from Illumina mNGS. The assembled genomic regions spanned 536,461 bp, or 9.9% of the bacterial genome. (C) Orthogonal confirmation of Bartonella henselae by dPCR of the DNA extract. Positive dPCR droplets (n=12) are seen in abscess fluid and the positive control consisting of sheared DNA from Bartonella henselae (ATCC). The negative control was water.

FIG. 10A-D shows length distributions of pathogen cfDNA in mNGS data. Analysis is performed on the 87 body fluid samples sequenced on both Illumina and nanopore platforms. (A) Diagram showing how original genomic DNA lengths are recovered. Paired-end sequencing data is aligned to either a human or microbial genome, followed by determination of fragment length from the start and end positions and construction of a read length histogram. (B) Histogram of average DNA lengths for human, bacterial, and fungal organisms obtained from mNGS data. Human DNA is observed to peak at the stereotypical 160 bp nucleosome footprint; both bacterial and fungal DNA are most abundant at sizes of <100 bp, but a higher molecular weight tail is observed extending to 500-600 bp. (C) Histogram of bacterial read lengths according to sequencing platform. Illumina and nanopore sequencing platforms produce different size distributions. (D) Length analysis of mNGS-positive, 16S-negative cases. Comparison of the length profiles of the 16S discordant bacteria cases (S31 and S88), 16S concordant bacteria cases (mean of S10, S36, S41, S69, S85, S128), and all bacteria mean. The pathogen cfDNA in cases S31 and S88 are more fragmented, with the vast majority of fragments <300 bp. The relative paucity of longer fragments could hinder 16S PCR amplification.

FIG. 11A-E provides a comparison of different threshold variables on the training set to calibrate the thresholds for each variable used. The final thresholds used are circled in each ROC chart. (A) Comparison of different minimal read thresholds for bacteria calling. Based on this data and prior selection of minimal reads, we selected a minimal of 3 reads for the validation set. (B) Comparison of using or not using a PCR Ct value normalization for bacteria calling. Normalization resulted in higher specificity and was used on the validation set. (C) Comparison of using a same-genus/same-family filter to decrease an informatics artifact where a pathogen burden is high and related species would appear at significant lower values. Using this filter improved specificity. (D) Comparison of different minimal read thresholds for fungal calling. We selected a minimal of 1 read based on the significantly higher sensitivity at the lowest threshold. (E) Comparison of using or not using a PCR Ct value normalization for fungal calling. Normalization resulted in higher specificity and was used on the validation set.

FIG. 12 provides 192 37mer barcode sequences of the disclosure.

### DETAILED DESCRIPTION

As used herein and in the appended claims, the singular forms “a,” “an,” and “the” include plural referents unless the context clearly dictates otherwise. Thus, for example, reference to “a body fluid” includes a plurality of such body fluids and reference to “the organism” includes reference to one or more organisms and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments only and is not intended to limit the scope of the disclosure, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means±1%.

As used herein, the term “amount” or “level” in reference to a targeted biomolecule, refers to a quantity of the targeted molecule that is detectable or measurable in a sample and/or control.

As used herein, the term “biological sample” includes any sample(s) that is taken from a subject which contains one or more targeted biomolecules described herein. Suitable samples in the context of the present disclosure include, for example, blood, plasma, serum, amniotic fluid, vaginal excretions, saliva, and urine. In a particular embodiment, biological samples used in a method disclosed herein comprise a blood plasma sample and a body fluid sample. In a further embodiment, biological samples used in a method disclosed herein comprise cell-free DNA (cfDNA) from body fluids.

Although PCR tests targeting the conserved 16S ribosomal RNA (rRNA) gene (“16S PCR”) and 28S-internal transcribed ribosomal gene spacer (“28S-ITS PCR”) regions of bacteria and fungi, respectively, have been developed, concerns have been raised regarding detection sensitivity. Failure or delay in diagnosing infections results in extended hospitalizations, readmissions, and increased mortality and morbidity. In addition, undiagnosed patients nearly always require empiric broad-spectrum therapy, with increased risk of adverse side effects and antimicrobial drug resistance.

Metagenomic next-generation sequencing (mNGS) enables detection of nearly all known pathogens simultaneously from clinical samples. Previous work in this area has focused on a single, generally non-purulent body fluid type, and few studies to date have demonstrated clinical validation and/or utility. Methodology and sample types are also highly variable, making it difficult to evaluate comparative performance across different studies. In particular, purulent fluids, which often suggest an infectious etiology, can be challenging to analyze by mNGS due to high human host DNA background, which can decrease assay sensitivity.

Methods exist to enrich for pathogen-specific reads from metagenomic data, such as differential lysis of human cells, but the scope of detection using these approaches is largely restricted to bacteria and/or fungi. Rapid identification of pathogens from infected body fluid compartments is important because empiric antimicrobial treatment is often suboptimal, contributing to increased morbidity and mortality. Most metagenomic studies have employed Illumina™ sequencing platforms, with sequencing run times exceeding 16 hours and overall sample-to-answer turnaround times of 48-72 hours. In contrast, nanopore sequencing (MinION™ sequencer by Oxford Nanopore Technologies) can detect microbes within minutes of starting sequencing and with a <6-hour turnaround time. Nanopore sequencing has been extensively used for genomic surveillance of emerging viruses, but clinical metagenomic applications of the technology for pathogen detection have been limited. One published study describes the use of a saponin-based differential lysis enrichment method for metagenomic nanopore sequencing-based detection of bacteria in respiratory infections with 96.6% sensitivity yet only 41.7% specificity.

Provided herein are simple, rapid, and universal methods for pathogen detection by mNGS analysis of cell-free DNA (cfDNA) from a variety of different body fluids, ranging from low-cellularity cerebrospinal fluid (CSF) to purulent fluids with high human host DNA content (e.g., abscesses). An innovative dual-use protocol, suitable for either Oxford Nanopore Technologies' nanopore or Illumina™ sequencing platforms, is used to evaluate the diagnostic accuracy of mNGS testing against traditional culture and PCR-based testing. A case series evaluating the performance of mNGS testing in 12 patients with culture- and PCR-negative body 95 fluids is described herein. For all cases, there was either high clinical suspicion for an infectious etiology or a confirmed microbiological diagnosis by orthogonal laboratory testing.

Described herein are rapid diagnostic assays for unbiased metagenomic detection of DNA-based pathogens from body fluids. Some advances underlying the approaches presented herein, include: (i) detection across a broad range of sample types, (ii) compatibility with input cfDNA concentrations varying across 6 orders of magnitude (100 pg/mL-100 ug/mL), (iii) a dual-use barcoding system enabling deployment on Illumina and nanopore sequencing platforms, and (iv) clinically validated bioinformatics pipelines for automated analysis and interpretation of mNGS data. Importantly, it was found that sensitivities and specificities for bacterial and fungal detection across Illumina and nanopore sequencing platforms were comparable. The potential utility of the methods of the disclosure are highlighted by detection of pathogens in 7 of 12 (58.3%) selected cases for which culture and PCR testing of the body fluid were negative, with subthreshold detection of pathogen reads in an additional two cases (9 of 12, 75%) (Table 11).

In the studies presented herein, mNGS testing failed to detect S. aureus at higher rates than other bacteria, a finding that was statistically significant for nanopore but not for Illumina sequencing. The lower sensitivity of S. aureus detection by nanopore sequencing was attributed to higher levels of human host background DNA. Notably, the median body fluid white blood cell (WBC) count for S. aureus was 70,250×10⁹/L (IQR 42,800-137,500), an approximately 100-fold increase over median WBC counts for other microorganisms (p<0.00001 by Mann-Whitney U-test). Other factors contributing to lower sensitivity for nanopore sequencing may be the lower read depths achieved in the current study and higher error rates relative to Illumina sequencing. These limitations are addressed by increasing average sequencing throughput per sample or making improvements in nanopore read accuracy over time.

The methods disclosed herein utilize pathogen-specific cfDNA sequences in body fluid supernatant. Intact pathogen DNA from high human DNA background samples, such as respiratory or joint fluids, can be obtained using differential lysis protocols. However, as the supernatant containing pathogen cfDNA is removed during the differential lysis protocol, such enrichment methods may not work as well for low cellularity samples such as plasma and CSF. Differential lysis can also hinder detection of other microorganisms such as viruses and parasites. In addition, these methods involve multiple steps of lysis and centrifugation, which can increase method complexity and prolong assay turnaround times. The methods disclosed herein also forego the use of mechanical processing steps such as bead-beating. Bead-beating may improve the detection of intact fungi and some bacteria containing rigid cell walls, but is laborious for routine use in the clinical laboratory and can reduce detection sensitivity by increasing host background from the release of human DNA.

While other studies have used metagenomic sequencing for pathogen detection in sepsis and pneumonia, the reported test specificities of 63% and 42.7% respectively, limiting broad clinical application, as it can be challenging to evaluate the clinical significance of false-positive results. In direct contrast, an overall specificity ranging from 83% to 100% was achieved using the methods and compositions of the disclosure.

Pathogen cfDNA analysis from blood has been used to diagnose deep-seated infections. However, bacterial DNA is often present at low levels in blood, with a lower quartile of 5 bacterial genome copies per mL in patients with sepsis. In matched pairs of samples, it was shown herein that there was an observed 160-fold higher pathogen cfDNA burden in body fluids. Similarly, tumor cfDNA is higher in adjacent body fluids than in blood. Higher levels of pathogen cfDNA in body fluids can increase analytical sensitivity and decrease sequencing depths required for accurate detection, thereby lowering the cost of testing. In addition, direct identification of a pathogen from a body fluid can localize the source of an infection, which is important to guiding definitive management and treatment.

In comparing mNGS with bacterial 16S or fungal 28S-ITS PCR, occult pathogens were detected solely by mNGS in 5 of 14 cases. False-negative 16S PCR results have been previously reported, and are generally attributed to suboptimal primer design or decreased assay sensitivity from background contamination. However, discordant results between 16S PCR and mNGS may also be due to short pathogen read lengths in cell-free body fluids. Notably, size ranges for bacterial 16S PCR amplicons span 300-460 nt, whereas those for fungal 28S-ITS PCR amplicons span 250-650 nt. Decreases in sensitivity due to fragmented cfDNA that are not amenable to long-read amplicon PCR have also been observed for detection of EBV virus in clinical samples.

The mNGS methods of the disclosure expand the scope of conventional diagnostic testing to multiple body fluid types. The achievable <6-hour turnaround time using nanopore sequencing may also be important for infections such as sepsis and pneumonia that demand a rapid response and timely diagnosis. The results presented herein indicate that mNGS testing methods disclosed herein are useful for a plurality of scenarios, including: (i) for identification of culture-negative or slow-growing pathogens, (ii) for diagnosis of rare or unusual infections that were not considered by the health care provider a priori, (iii) as a first-line test in critically ill patients, and (iv) as an early alternative to the large number of send out tests that would otherwise be ordered as part of the diagnostic workup.

The studies presented herein have focused on clinical development and validation of metagenomic sequencing technologies, including pathogen detection and gene expression profiling, to diagnose infections in clinical samples from patients. There are key advantages and disadvantages regarding the choice of sequencing technologies for the metagenomic sequencing approach. For instance, nanopore sequencing (currently available on the MinION™, GridION™, or PromethION™ instruments by Oxford Nanopore Technologies™, or ONT) enables longer reads and “real-time” sequencing analysis; the latter aspect enables more rapid sequencing protocols and shorter turnaround times, albeit with lower throughput and higher error rates. Illumina™ sequencing, in contrast, has much higher throughput (number of reads per given unit time) and lower costs, albeit at greater turnaround times.

Presented herein is the development and validation of a hybrid approach and barcoding schemes in which sequencing libraries can be constructed from samples that would be compatible (e.g., can be sequenced) on a variety of different sequencing platforms. Most sequencing technologies utilize “adapter-ligation” protocols for barcoding and sequencing, whereby an indexed adapter is attached to the end(s) of free DNA or cDNA molecules in order to barcode multiplexed samples and facilitate a subsequent sequencing reaction. The hybrid approach for use with ONT and Illumina platforms can use an adapter-ligation approach coupled to the same or different-sized barcodes (e.g., 37mers for the ONT and 8mers—the first or last 8 bases of the 37mer for Illumina) to generate barcoded, dual- or singly-indexed libraries that are compatible with both platforms.

In a particular embodiment, the disclosure provides at least one, typically a set including 2, 3, 4 or more pairs of a barcode Xmer (wherein X is an integer selected 15, 16, 17, 18, 19, 20, 21, 22, 23, 24, 25, 26, 27, 28, 29, 30, 31, 32, 33, 34, 35, 36, 37, 38, 39, 40, 41, 42 or more) barcodes comprising an index (e.g., dual indexes comprising a first domain or bridge domain index and a second domain or nanopore domain index) that is configured for use in multiple types of next generation sequencing technologies, wherein the Levenshtein distance has been maximized between each pair of Xmer barcodes in order to minimize barcode “crosstalk”; wherein the first or last, e.g., 4 to 9 nucleotides (4-9mer) of each pair of Xmer barcodes is compatible with a next generation sequencing technology that utilizes bridge amplification (e.g., iSeq100, MiniSeq, MiSeq, HiSeq, NovaSeq, and NextSeq from Illumina™), and wherein at least a minimum Levenshtein distance between each pair of, e.g., 4-9mers is utilized; wherein at least a minimum Levenshtein distance between each pair of Xmer barcodes is used so that the Xmer barcode is compatible with a next generation sequencing technology that utilizes nanopores (e.g., Flongle, MinION, MinION Mk1C, GridION, and promethION from Oxford Nanopore Technologies™). In a further embodiment, the Xmer barcodes are comprised of the, e.g., 4-9mer and, e.g., 3 sets of 10mer barcodes that concatenated together to form, for example, a Xmer of 33-39 nucleotides, wherein the last nucleotide is removed to form the Xmer barcodes of 32-38 nucleotides. In regards to Levenshtein distance, the Levenshtein distance can be computed using the methods presented herein, or the Levenshtein distance calculations described in detailed in Bushmann et al., (“Levenshtein error-correcting barcodes for multiplexed DNA sequencing.” BMC Bioinformatics 14: 272 (2013)), the disclosure of which is incorporated herein in full.

It should be recognized that the second ‘nanopore’ domain index can completely overlap and encompass the first ‘bridge’ domain index. The overall length can have a higher upper limit, such as 160 nucleotides. The exemplary oligonucleotides described in the Examples below used two 37mers, for a total of 74 nucleotides. Moreover, the first ‘bridge’ domain can go up to two 12mers, so the minimum can be high at 24 or 25 nucleotides total. Although the Examples, use a 37mer, an exact 37mer is not necessary, e.g., 36mer or 38mer will also work. The second ‘nanopore’ barcode index can be at least a total of 24 nucleotides (all locations combined). Alternatively, the second ‘nanopore’ barcodes are at least double in length the size of the bridge amplification barcodes. In addition, paired barcodes are not required. The barcodes can be arbitrarily shifted between the two sides, all the way on one side or the other, to effectively have single-end barcodes. Index barcodes can also be easily shifted into other locations—currently, in the Illumina and nanopore configuration, there are 4 convention locations, so the total can be quadruple barcodes rather than paired. In addition, although the Examples below used an 8mer first ‘bridge’ domain index it does not have to be a precise 8mer. For example, bridge amplification systems such as that on Illumina systems also use 6mers, 7mers, 8mers or 9mers.

In a particular embodiment, the disclosure provides for a set of oligonucleotides comprising a set of Xmer barcodes (e.g., a 37mer) disclosed herein. In a further embodiment each Xmer barcode is located between a pair of sequencing adaptors. In yet a further embodiment, the pair of sequencing adaptors have sequences selected from (i) or (ii):
(i) CAAGCAGAAGACGGCATACGAGAT (SEQ ID NO:1), and GTGACTGGAGTTCAGACGTGTGCTCTTCCGATC*T (SEQ ID NO:2); or
(ii) AATGATACGGCGACCACCGAGATCTACAC (SEQ ID NO:3), and ACACTCTTTCCCTACACGACGCTCTTCCGATC*T (SEQ ID NO:4), wherein * indicates a phosphorothioate bond between the nucleotides. In another embodiment, the set of oligonucleotides are PCR primers used for sequencing library barcoding.

In a certain embodiment, the disclosure also provides a sequencing library comprising a set of paired Xmer barcodes (wherein X is between 15 and 42 nt) disclosed herein. In a further embodiment, the sequencing library is used for an application selected from: pathogen discovery, environmental metagenomics, de novo genome assembly, whole-exome sequencing, transcriptomics sequencing, targeted gene panel sequencing or whole-genome resequencing. In a further embodiment, the sequencing library is generated using a library preparation kit. In yet a further embodiment, the library preparation kit is from Illumina, Inc (e.g., AmpliSeq™ kits, COVIDSeq™ kit, Illumina DNA prep kits, Illumina RNA prep kits, Nextera™ Kits, SureCell WTA™ Kits, TruSeq™ kits, and TruSight™ kits).

In a particular embodiment, the disclosure also provides a method for rapid pathogen detection in a sample using metagenomic next-generation sequencing (mNGS), comprising: obtaining one or more samples comprising cell-free DNA (cfDNA); generating a plurality of sequencing read data comprising a Xmer barcode (wherein X is between 15 and 42 nt) from a set of paired Xmer barcodes wherein the Levenshtein distance has been maximized between each pair of Xmer barcodes in order to minimize barcode “crosstalk”; wherein the first or last 4 to 9 nucleotides (4-9mer) of each pair of Xmer barcodes is compatible with a next generation sequencing technology that utilizes bridge amplification, and wherein at least a minimum Levenshtein distance between each pair of 4-9mers is utilized and wherein at least a minimum Levenshtein distance between each pair of Xmer barcodes is used so that the Xmer barcode is compatible with a next generation sequencing technology that utilizes nanopores; performing metagenomic analysis on the plurality of sequencing read data using a clinical bioinformatics software pipeline that can rapidly analyze sequencing read data for pathogenic DNA; identifying pathogen(s) in the one or more samples based upon the metagenomic analysis of the sequencing read data. In another embodiment, the one or more samples comprises a body fluid sample from a subject. In yet another embodiment, the body fluid sample is a purulent body fluid sample. In a certain embodiment, the body fluid sample is selected from cerebrospinal fluid, urine, semen, pericardial fluid, pleural fluid, peritoneal fluid, synovial fluid, amniotic fluid, fetal fibronectin, saliva, sweat, eye vitreous humor, eye aqueous humor, bronchoalveolar lavage fluid, breast milk, bile, and ascites fluid. In another embodiment, the one or more samples further comprise a blood serum sample. In yet another embodiment, the next-generation sequencing comprises sequencing technology that utilizes bridge amplification. In a further embodiment, the next-generation sequencing comprises or further comprise sequencing technology that utilizes nanopores. In yet a further embodiment, the next-generation sequencing comprises sequencing technology that utilizes bridge amplification and sequencing technology that utilizes nanopores. In another embodiment, the clinical bioinformatics software pipeline that can rapidly analyze sequencing read data for pathogenic DNA is SURPI+ or SURPIrt. In a further embodiment, the pathogen(s) comprise one or more pathogenic bacteria. In an alternate embodiment, the pathogen(s) comprise one or more pathogenic fungi.

Methods using the hybrid approach described herein allows for short read, high-throughput, slower sample-to-sequence technologies, such as Illumina, to be performed simultaneously with long read, lower-throughput, rapid sequencing technologies, such as ONT. The methods disclosed herein by using such a hybrid approach, can leverage key advantages of each sequencing technology (e.g., ONT nanopore sequencing—speed; Illumina sequencing—throughput). In the studies presented herein, the hybrid approach described herein was successfully run with 37mer barcoding for ONT nanopore sequencing and 8mer barcoding for Illumina sequencing. Accordingly, the disclosure has provided methodologies where two or more sequencing platforms can be used simultaneously and successfully for metagenomic analysis in a number of applications, including, but not limited to, clinical diagnosis, pathogen discovery, de novo genome assembly, whole-exome sequencing, targeted gene panel sequencing, transcriptome profiling, and whole-genome resequencing.

Accordingly, the disclosure further provides for integrated assays to simultaneously use multiple sequencing platforms for metagenomic analysis, such as assay kits. Such assay kits can be used for applications, including but not limited to, clinical diagnosis with initial sequencing for rapid diagnosis (e.g., ONT platform) followed by more complete reflex sequencing for high sensitivity (e.g., Illumina platform); generating hybrid libraries for all sequencing applications, including, but not limited to, pathogen discovery, environmental metagenomics, de novo genome assembly whole-exome sequencing, transcriptomics sequencing (e.g., RNA-Seq); targeted gene panel sequencing; and whole-genome resequencing (e.g., cancer genome sequencing). Such assay kits provide a “one stop” kit to perform metagenomic analysis on samples, include primers, sequencing reagents, analysis software, etc. In a particular embodiment, the kit comprises, consists essentially of, or consists of dual use barcode primers that have been designed using the methods disclosed herein that can be used in both Illumina and Oxford Nanopore Technologies instruments. In another embodiment, a kit described herein is used to determine pathogenic microorganism(s) in patient sample(s) using the methods disclosed herein.

The assay kit will comprise a plurality of detection/quantification tools specific to each targeted biomolecule detected by the kit (e.g., pathogenic nucleic acid). Many of the targeted biomolecules disclosed herein comprise DNA, which may be detected by next generation sequencing and like technologies. The detection/quantification tools may comprise a set of dual use barcode primers, each barcode primer directed to the selective amplification by NGS of a targeted biomolecule(s) in a sample.

In yet another embodiment, the assay kits of the disclosure further comprise reagents or enzymes which can be used for next generation sequencing and like technologies. Assay kits may further comprise elements such as reference DNAs (e.g., positive and negative controls), washing solutions, buffering solutions, reagents, printed instructions for use, and containers.

The following examples are intended to illustrate but not limit the disclosure. While they are typical of those that might be used, other procedures known to those skilled in the art may alternatively be used.

### Examples

Sample selection and processing. All body fluid samples were obtained from patients at the University of California San Francisco (UCSF) hospitals and clinics for three years. The study only used residual body fluid samples after standard-of-care clinical laboratory testing was performed. Body fluid samples were collected in sterile tubes or using swabs as part of routine clinical care and included abscess, joint, peritoneal, pleural, cerebrospinal, urine, bronchoalveolar lavage and other fluids (see Table 1). Swabs were stored in charcoal gel columns (Swab Transport Media Charcoal 220122, BD) and reconstituted in 0.5 mL of Universal Transport Media (350C, Copan Diagnostics, Murrieta, CA); the media liquid was subsequently used for culture, PCR, and mNGS analyses. Cultures for bacteria, fungi, and AFB from body fluid samples were done in-house at UCSF. Clinical 16S rDNA and 28S-ITS PCR for bacterial and fungal detection were performed by a reference laboratory at the University of Washington. Residual samples were stored at 4° C. and tested within 14 days of collection or centrifuged at 16,000 relative centrifugal force for 10 minutes and the supernatant stored at −80° C. until time of extraction.

Plasma samples were obtained by collecting blood from hospitalized patients as part of routine clinical testing into EDTA Plasma Preparation Tubes (BD) or standard EDTA Tubes (BD). The tubes were centrifuged (4000-6000 rcf for 10 minutes) within 6 hours, and plasma was isolated from the buffy coat and red cells. The plasma component was further aliquoted and centrifuged at 16,000 rcf for 10 minutes in microcentrifuge tubes. Plasma samples were stored at −80° C. until the time of extraction.

In the study of test performance, body fluids samples were included if they were culture positive or PCR positive for bacterial or fungal pathogen(s) with pathogen(s) identified to genus/species level. Body fluids from patients with ambiguous laboratory findings (e.g., a positive culture that was judged clinically to be a contaminant) or from patients with an established infectious diagnosis and already receiving targeted treatment at the time of body fluid collection were excluded. Negative control body fluid samples were selected from patients who had clear alternative non-infectious diagnoses (e.g., cancer, trauma) and negative for infection by culture and clinical adjudication (CYC and WG).

In the series of 12 cases, body fluid samples were included if (i) they were culture and PCR negative and (ii) from a patient with a microbiologically established infection (by orthogonal testing such as serology or testing of a different body fluid/tissue) or clinically probable infection based on review of the clinical charts by an infectious disease specialist (CYC) and clinical pathologist (WG) (Table 11).

TABLE 1
Clinical characteristics and INGS results of all cases used in the accuracy study.

| | | | | Blood Culture | |
|---|---|---|---|---|---|
| Sample | Inclusion | Gold Standard - | Tests | (same admission, | Sample |
| # | Criteria | Culture/16S | Performed | within +/−3 days) | Type |
| S1 | Positive | Staphylococcus | Bacterial | n/a | Abscess |
| | Culture | aureus | Culture | | |
| | (Bacteria) | | | | |
| S2 | Positive | Enterobacter | Bacterial | negative x1 | CSF |
| | Culture | aerogenes | Culture | | |
| | (Bacteris) | | | | |
| S3 | Positive | Group B | Bacterial | negative x2 | Joint Fluid |
| | Culture | Streptococcus | Culture | | |
| | (Bacteria) | | | | |
| S4 | Positive | Candida | Bacterial | n/a | CSF |
| | Culture | parapsilosis | Culture | | |
| | (Bacteria) | | | | |
| S5 | Positive | Staphylococcuss | Bacterial | Staphylococcus | Joint Fluid |
| | Culture | aureus | | aureus x1 | |
| | (Bacteria) | | | | |
| S6 | Positive | Pseudomonas | Bacterial | n/a | Abscess |
| | Culture | aeruginosa | and Fungal | | |
| | (Bacteria) | | Culture | | |
| S7 | Negative | negative | Cytology | n/a | Pleural |
| | Control | | (malignant); | | Fluid |
| | (cause: | | Bacterial | | |
| | cancer) | | Culture | | |
| S8 | Positive | Sraphylococcus | Bacterial | n/a | BAL |
| | Culture | aureus | and Fungal | | |
| | (Bacteria) | | Culture | | |
| S9 | Positive | Staphylococcus | Bacterial | Staphylococcus | Pleural |
| | Culture | aureus | Culture | aureus x10+ | Fluid |
| | (Bacteria) | | | | |
| S10 | Positive 16S | Haemophilus | 16S, | Haemophilus | BAL |
| | | infuenzae | Bacterial, | influenzae x1 | |
| | | | Fragal, | (OSH) | |
| | | | and AFB | | |
| | | | Culture | | |
| S11 | Positive | Staphylococcus | Bacterial | n/a | Peritoneal |
| | Culture | aureus | Culture | | Fluid |
| | (Bacteria) | | | | |
| S12 | Positive | Staphylococcus | Bacterial | negative x2 | Joint Fluid |
| | Culture | aureus | Culture | | |
| | (Bacteria) | | | | |
| S13 | Positive | Staphylococcus | Bacterial | negative x2 | Joint Fluid |
| | Culture | aureus | Culture | | |
| | (Bacteria) | | | | |
| S14 | Positive | Pseudomonas | Bacterial | n/a | Vitreous |
| | Culture | aeruginosa | and Fungal | | Fluid |
| | (Bacteria) | | Culture | | |
| S15 | Positive | Staphylococcus | Bacterial | n/a | BAL |
| | Culture | aureus | and Fungal | | |
| | (Bacteria) | | and AFB | | |
| | | | Culture | | |
| S16 | Negative | negative | Bacterial | n/a | CSF |
| | Control | | and Fungal | | |
| | (cause: | | Culture | | |
| | cancer) | | | | |
| S17 | Positive | Serratia | Bacterial | 1 blood culture | Pleural |
| | Culture | marcescens | and Fungal | 2 days latex: | Fluid |
| | (Bacteria) | | and AFB | Candida | |
| | | | Culture | tropicalis. | |
| | | | | Previous Serratia | |
| | | | | bacteremia this | |
| S18 | Positive | Staphylococcus | Bacterial | negative | CSF |
| | Culture | epidermidis | Culture | | |
| | (Bacteria) | group | | | |
| S19 | Positive | Serratia | Bacterial | Central line 3 | Pleural |
| | Culture | marcescens | and Fungal | days ago positive | Fluid |
| | (Bacteria) | | Culture | for Candida | |
| | | | | tropicalis | |
| S20 | Positive | Enterococcus | Bacterial | n/a | CSF |
| | Culture | faecium | Culture | | |
| | (Bacteria) | | | | |
| S21 | Positive | Enterococcus | Bacterial | negative | CSF |
| | Culture | faecium | Culture | | |
| | (Bacteria) | | | | |
| S22 | Positive | Candida | Bacterial | n/a | Peritoneal |
| | Culture | albicans, | Culture | | Fluid |
| | (Bacteria) | Candida | | | |
| | | glabrata | | | |
| S23 | Positive | Staphylococcus | Bacterial | negative | Abscess |
| | Culture | aureus, | Culture | | |
| | (Bacteria) | Escherichia | | | |
| | | coli | | | |
| S24 | Positive | Staphylococcus | Bacterial | negative | Pleural |
| | Culture | aureus | and AFB | | Fluid |
| | (Bacteria) | | Culture | | |
| S25 | Negative | negative | Bacterial | n/a | Joint Fluid |
| | Control | | Culture | | |
| | (cause: | | | | |
| | trauma) | | | | |
| S26 | Positive | Enterococcus | Bacterial | negative x2 | Pleural |
| | Culture | faecium, | Culture | 2 days later | Fluid |
| | (Bacteria) | Candida | | | |
| | | albicans | | | |
| S27 | Positive | Candida | Bacterial | negative | Abscess |
| | Culture | glabrata | Culture | | |
| | (Bacteria) | | | | |
| S28 | Positive | Streptococcus | Bacterial | Data unavailable | Abscess |
| | Culture | pyogenes | Culture + | | |
| | (Bacteria) | | <not | | |
| | | | known> | | |
| S29 | Positive | Enterobacter | Bacterial | n/a | Abscess |
| | Culture | cloacae | Culture | | |
| | (Bacteria) | complex, | | | |
| | | Candida | | | |
| | | albicans | | | |
| S30 | Positive | Staphylococcus | Bacterial | Same organism 2 | Joint Fluid |
| | Culture | aureus | Culture | and 3 days prior | |
| | (Bacteria) | | | | |
| S31 | Positive 16S | Streptococcus | 16S | negative | Pleural |
| | | mitis group | Bacterial | | Fluid |
| | | | Culture | | |
| S32 | Positive | Staphylococcus | Bacterial | Multiple blood | Abscess |
| | Culture | aureus | Culture | cultures positive | |
| | (Bacteria) | | | for same | |
| | | | | organism | |
| S33 | Negative | negative | Bacterial | n/a | CSF |
| | Control | | and AFB | | |
| | (cause: | | Culture | | |
| | cancer) | | | | |
| S34 | Positive | Streptococcus | Bacterial | negative or | Pleural |
| | Culture | anginosus | Culture | Corynebacterium | Fluid |
| | (Bacteria) | group | | spp NOS | |
| S35 | Negative | negative | Bacterial | negative | Pleural |
| | Control | | and AFB | | Fluid |
| | (cause: | | Culture | | |
| | cancer) | | | | |
| S36 | Positive 16S | Staphylococcus | 16S, | negative | Abscess |
| | | aureus | Bacterial | | |
| | | | and Fungal | | |
| | | | Culture | | |
| S37 | Positive | Enterococcus | Bacterial | Enterococcus | Perihepatic |
| | Culture | faecalis, | Culture | faecalis x2 | Fluid |
| | (Bacteria) | Entercoccus | | a day prior | |
| | | faecium, | | | |
| | | Candida | | | |
| | | knaei | | | |
| S38 | Positive | Staphylococcus | Bacterial | negative | Abscess |
| | Culture | aureus | Culture | | |
| | (Bacteria) | | | | |
| S39 | Positive | Staphylococcus | Bacterial | n/a | Joint Fluid |
| | Culture | aureus | Culture | | |
| | (Bacteria) | | | | |
| S40 | Negative | negative | Bacterial | negative | Pleural |
| | Control | | Culture | | Fluid |
| | (cause: | | | | |
| | cancer) | | | | |
| S41 | Positive 16S | Streptococcus | 16S, | negative | Pleural |
| | | pyogenes | Bacterial | | Fluid |
| | | | and Fungal | | |
| | | | and AFB | | |
| | | | Culture | | |
| S42 | Positive | Escherichia | Bacterial | negative | Abscess |
| | Culture | coli | Culture | (2x positive | |
| | (Bacteria) | | | 5 days ago) | |
| S43 | Positive | Staphylococcus | Bacterial | Positive same | Joint Fluid |
| | Culture | aureus | Culture | organism | |
| | (Bacteria) | | | | |
| S44 | Positive | Staphylococcus | Bacterial | negative | Abscess |
| | Culture | aureus | Culture | | |
| | (Bacteria) | | | | |
| S45 | Positive | Staphylococcus | Bacterial | negative | Abscess |
| | Culture | aureus | Culture | | |
| | (Bacteria) | | | | |
| S46 | Positive | Staphylococcus | Bacterial | n/a | Joint Fluid |
| | Culture | aureus | Culture | | |
| | (Bacteria) | | | | |
| S47 | Positive | Excherichia | Bacterial | Data unavailable | Peritoneal |
| | Culture | coli, | Culare + | | Fluid |
| | (Bacteria) | Klebsiella | <not | | |
| | | pneumaniae | known> | | |
| S48 | Positive | Staphylococcus | Bacterial | n/a | Abscess |
| | Culture | aureus | and Fungal | | |
| | (Bacteria) | | and AFB | | |
| | | | Culture | | |
| S49 | Positive | Enterococcus | Bacterial | negative | Swab |
| | Culture | spp, | Culture | | |
| | (Bacteria) | Candida | | | |
| | | albicans | | | |
| S50 | Positive | Escherichia | Bacterial | negative | Joint Fluid |
| | Culture | coli | Culture | | |
| | (Bacteria) | | | | |
| S51 | Positive | Aspergillus | Fungal | negative | BAL |
| | Culture | fumigatus | Culture | | |
| | (Fungal) | | and ITS | | |
| | | | sequencing | | |
| S52 | Positive | Staphylococcus | Bacterial | negative | Joint Fluid |
| | Culture | aureus | and Fungal | | |
| | (Bacteria) | | and AFB | | |
| | | | Culture | | |
| S53 | Positive | Klebsiella | Bacterial | 2 days ago had | Abscess |
| | Culture | pneumaniae, | Culture | Citrobacter | |
| | (Bacteria) | Citrobacter | | freundi | |
| | | freundii | | complex x2. | |
| | | complex | | | |
| S54 | Positive | Escherichia | Bacterial | negative | Subgaleal |
| | Culture | coli | and Fungal | | Fluid |
| | (Bacteria) | | Culture | | |
| S55 | Positive | Staphylococcus | Bacterial | Same organism | Joint Fluid |
| | Culture | aureus | Culture | same day and | |
| | (Bacteria) | | | later days | |
| S56 | Positive | Klebsiella | Bacterial | negative | Peritoneal |
| | Culture | pneumoniae | and Fungal | | Fluid |
| | (Bacteria) | | Culture | | |
| S57 | Positive | Enterococcus | Bacterial | negative | Peritoneal |
| | Culture | faecium | Culture | | Fluid |
| | (Bacteria) | | | | |
| S58 | Positive | Aspergillus | Bacterial | negative | Pleural |
| | Culture | fumigatus | and Fungal | | Fluid |
| | (Fungal) | | and AFB | | |
| | | | Culture | | |
| S59 | Positive | Pseudomonas | Bacterial | negative | Peritoneal |
| | Culture | aeruginosa, | and Fungal | | Fluid |
| | (Bacteria) | Candida | and AFB | | |
| | | glabrata, | Culture | | |
| | | Candida | | | |
| | | krusei | | | |
| S60 | Positive | Pseudomonas | Bacterial | n/a | Pleural |
| | Culture | aeruginosa | and AFB | | Fluid |
| | (Bacteria) | | Culture | | |
| S61 | Positive | Staphylococcus | Bacterial | n/a | Joint Fhud |
| | Culture | lugdimensis | Culture | | |
| | (Bacteria) | | | | |
| S62 | Positive | Escherichia | Bacterial | Same organism | Urine |
| | Culture | coli | Culture | x2 | |
| | (Bacteria) | | | | |
| S63 | Positive | Staphylococcus | Bacterial | n/a | Joint Fluid |
| | Culture | lugdimensis | Culture | | |
| | (Bacteria) | | | | |
| S64 | Positive | Mycoplasma | 16S on | negative | Peri-graft |
| | Culture | hominis | isolate on | | Fluid |
| | (Bacteria) | | Bacterial | | Swab |
| | | | Culture only | | |
| S65 | Positive 16S | Streptococcus | 16S and | negative | Peritoneal |
| | | pyogenes | Bacterial | | Fluid |
| S66 | Negative | negative | Bacterial | n/a | Joint Fluid |
| | Control | | and Fungal | | |
| | (cause: post | | Culture | | |
| | surgical | | | | |
| | chronic | | | | |
| | synovitis) | | | | |
| S67 | Negative | negative | Bacterial | n/a | Joint Fluid |
| | Control | | and Fungal | | |
| | (cause: post | | Culture | | |
| | surgical | | | | |
| | chronic | | | | |
| | synovitis) | | | | |
| S68 | Negative | negative | Bacterial | n/a | Joint Fluid |
| | Control | | and Fungal | | |
| | (cause: post | | Culture | | |
| | surgical | | | | |
| | chronic | | | | |
| | synovitis) | | | | |
| S69 | Positive 16S | Mycobacterium | 16S, | negative | Abscess |
| | | tuberculosis | Bacterial | | |
| | | complex | and Fungal | | |
| | | | and AFB | | |
| | | | Culture | | |
| S70 | Positive | Staphylococcus | Bacterial | n/a | Joint Fluid |
| | Culture | aureus | Culture | | |
| | (Bacteria) | | | | |
| S71 | Positive | Staphylococcus | Bacterial | negative | Abscess |
| | Culture | aureus | Culture | | |
| | (Bacteria) | | | | |
| S72 | Positive | Candida | Bacterial | negative | Peritoneal |
| | Culture | albicans | Culture | | Fluid |
| | (Bacteria) | | | | |
| S73 | Positive | Staphylococcus | Bacterial | Positive same | Anterior |
| | Culture | aureus | Culture | organism 2 | Mediastinal |
| | (Bacteria) | | | days ago | Fluid |
| S74 | Positive | Staphylococcus | Bacterial | negative | Peritoneal |
| | Culture | aureus | Culture | | Fluid |
| | (Bacteria) | | | | |
| S75 | Positive | Streptococcus | Bacterial | negative | Abscess |
| | Culture | mitis group | Culture | | |
| | (Bacteria) | | | | |
| S76 | Positive | Escherichia | Bacterial | Positive same | Peritoneal |
| | Culture | coli | Culture | organism | Fluid |
| | (Bacteria) | | | | |
| S77 | Positive | Candida | Bacterial | n/a | Peritoneal |
| | Culture | albicans | Culture | | Fluid |
| | (Bacteria) | | | | |
| S78 | Positive | Escherichia | Bacterial | negative | Urine |
| | Culture | coli | Culture | | |
| | (Bacteria) | | | | |
| S79 | Positive | Coccidioides | Bacterial | negative | Chest |
| | Culture | immitis | and Fungal | | Mass |
| | (Fungal) | | Culture | | |
| S80 | Positive | Coccidioides | Bacterial | negative | Chest |
| | Culture | immitis | and Fungal | | Mass |
| | (Fungal) | | Culture | | Fluid |
| S81 | Positive | Streptococcus | Bacterial | negative | Wound |
| | Culture | pyogenes | Culture | | Swab |
| | (Bacteria) | | | | |
| S82 | Negative | negative | Bacterial | negative (1 | Pleural |
| | Control | | Culture | Propionibacterium | Fluid |
| | (cause: | | | acnes) | |
| | cancer) | | | | |
| S83 | Positive | Aspergillus | Bacterial | negative | BAL |
| | Culture | terreus, | and Fungal | | |
| | (Fungal) | Aspergillus | and AFB | | |
| | | fumigatus | Culture | | |
| S84 | Negative | negative | Bacterial | negative | Pleural |
| | Control | | Culture | | Fluid |
| | (cause: | | | | |
| | cancer) | | | | |
| S85 | Positive | Streptococcus | Bacterial | negative | Abscess |
| | Culture | pyogenes | Culture, 16S | | |
| | (Bacteria) | | | | |
| S86 | Positive | Staphylococcus | Bacterial | Staphylococcus | Heel Fluid |
| | Culture | aureus | Culture | aureus | Swab |
| | (Bacteria), | | | | |
| | not run on | | | | |
| | nanopore | | | | |
| S87 | Positive | Finegoldia | Bacterial | negative | Abscess |
| | Culture | magna | Culture | | |
| | (Bacteria), | (Anaerobic) | | | |
| | not run on | | | | |
| | nanopore | | | | |
| S88 | Suspected | negative | Bacterial | negative | CSF |
| | culture FN: | but highly | Culture, 16S | | |
| | positive | probable | | | |
| | culture from | infection | | | |
| | brain surgery | | | | |
| | 2 days later | | | | |
| S89 | Suspected | negative | Bacterial | n/a | Abscess |
| | culture FN: | but highly | Culture | | |
| | bowel | probable | | | |
| | perforation | infection | | | |
| S90 | Suspected | negative | Bacterial | Streptococcus | Pleural |
| | culture FN: | but highly | and Fungal | pneumoniae | Fluid |
| | pneumonia | probable | and AFB | 3 days ago | |
| | and | infection | Culture | | |
| S91 | Suspected | negative | Bacterial | Blood Cx was | Pleural |
| | culture FN: | but highly | Culture | Group A | Fluid |
| | probable | probable | | Streptococcus | |
| | pneumonia | infection | | 4 days ago | |
| | and | | | | |
| | bacteremia | infection | | | |
| S92 | Suspected | negative but | Bacterial | negative | BAL |
| | culture FN: | highly | and Fungal | | |
| | probable | probable | and AFB | | |
| | invasive | infection | Culture | | |
| | aspergillosis | | | | |
| S93 | Positive | Nocardia | Bacterial | negative | Peritoneal |
| | Culture | farcinica | and Fungal | | Fluid |
| | (Bacteria) | | and AFB | | |
| | | | Culture | | |
| S94 | Positive | Caccidioides | Bacterial | negative | CSF |
| | Culture | immitis | and Fungal | | |
| | (Fungal) | | Culture | | |
| S95 | Positive | Staphylococcus | Bacterial | positive for | Joint Fluid |
| | Culture | aureus | and Fungal | same organism | |
| | (Bacteria) | | and AFB | | |
| | | | Culture | | |
| S96 | Positive | Staphylococcus | Bacterial | negative | Peritoneal |
| | Culture | aureus | Culture | | Fluid |
| | (Bacteria) | | | | |
| S97 | Positive | Candida | Bacterial | negative | CSF |
| | Culture | glabrata | and Fungal | | |
| | (Fungal) | | Culture | | |
| S98 | Positive | Group A | Bacterial | negative | Peritonsilar |
| | Culture | Streptococcus | Culture | | Drainage |
| | (Bacteria) | | | | |
| S99 | Positive | Streptococcus | Bacterial | 2x same | CSF |
| | Culture | pneumoniae | Culture | organism | |
| | (Bacteria) | | | | |
| S100 | Positive | Staphylococcus | Bacterial | n/a | Peritoneal |
| | Culture | epidermidis | and Fungal | | Fluid |
| | (Bacteria) | | and AFB | | |
| | | | Culture | | |
| S101 | Positive | Candida | Bacterial | negative | Back |
| | Culture | albicans | and Fungal | | Fluid |
| | (Fungal) | | and AFB | | |
| | | | Culture | | |
| S102 | Positive | Staphylococcus | Bacterial | negative | Joint Fluid |
| | Culture | aureus | Culture | | |
| | (Bacteria) | | | | |
| S103 | Positive | Escherichia | Bacterial | positive for | CSF |
| | Culture | coli | Culture | same organism | |
| | (Bacteria) | | | | |
| S104 | Positive | Candida | Bacterial | n/a | Peritoneal |
| | Culture | glabrata | and Fungal | | Fluid |
| | (Fungal) | | aod AFB | | |
| | | | Culture | | |
| S105 | Positive | Cryptococcus | Bacterial | same organism | CSF |
| | Culture | neoformans | and Fungal | | |
| | (Fungal) | | and AFB | | |
| | | | Culture | | |
| S106 | Positive | Candida | Bacterial | 3 days after: | CSF |
| | Culture | parasilopsis | Culture | negative | |
| | (Fungal) | | | | |
| S107 | Positive | Candida | Bacterial | 1 day prior: | CSF |
| | Culture | parasilopsis | Culture | negative | |
| | (Fungal) | | | | |
| S108 | Positive | Candida | Bacterial | 5 days prior: | CSF |
| | Culture | parasilopsis | Culture | negative | |
| | (Fungal) | | | | |
| S109 | Positive | Cryptococcus | Bacterial | same organism | CSF |
| | Culture | neoformans | and Fungal | | |
| | (Fungal) | | and AFB | | |
| | | | Culture | | |
| S110 | Positive | Aspergillus | Bacterial | 2 days prior: | BAL |
| | Culture | spp, mixer | and Fungal | negative | |
| | (Fungal) | morphotypes | and AFB | | |
| | | (moderata A. | Culture | | |
| | | niger, rare A. | | | |
| | | flavus, rare A. | | | |
| | | fumigatus), Few | | | |
| | | Oronosal flora | | | |
| S111 | Positive | Staphylococcus | Bacterial | 1 and 5 days | Abscess |
| | Culture | aureus | Culture | after: same | |
| | (Bacteria) | | | organism | |
| S112 | Positive | Enterococcus | Bacterial | negative | Peritoneal |
| | Culture | faecium | and Fungal | | Fluid |
| | (Bacteria) | | and AFB | | |
| | | | Culture | | |
| S113 | Positive | Staphylococcus | Bacterial | negative | Joint Fluid |
| | Culture | aureus | Culture | | |
| | (Bacteria) | | | | |
| S114 | Positive | Coccidioides | Bacterial | negative | Pleural |
| | Culture | immitis | and Fungal | | Fluid |
| | (Fungal) | | and AFB | | |
| | | | Culture | | |
| S115 | Positive | Group 4 | Bacterial | n/a | Knee |
| | Culture | Streptococcus, | and Fungal | | Swab |
| | (Bacteria) | Corynebacterium | and AFB | | |
| | | diphthering | Culture | | |
| S116 | Positive | Cryptococcus | Bacterial | negative | BAL |
| | Culture | neoformens | and Fungal | | |
| | (Fungal) | | and AFB | | |
| | | | Culture | | |
| S117 | Positive | Salmonella | Bacterial | negative | Abscess |
| | Culture | typhi group D | and Fungal | | |
| | (Bacteria) | | and AFB | | |
| | | | Culture | | |
| S118 | Positive | Mycobacterium | Bacterial | n/a | FNA |
| | Culture | tuberculosis | and Fungal | | |
| | (Bacteria) | complex | and AFB | | |
| | | | Culture | | |
| S119 | Positive | Escherichia | Bacterial | same day 2x | Peritoneal |
| | Culture | coli | Culture | same organismi | Fluid |
| | (Bacteria) | | | | |
| S120 | Positive | Enterobacter | Bacterial | negative | Urine |
| | Culture | cloacae, | and Fungal | | |
| | (Fungal) | Candida | and AFB | | |
| | | albicans | Culture | | |
| S121 | Positive | Coccidioides | Bacterial | negative | CSF |
| | Culture | immitis | and Fungal | | |
| | (Fungal) | | and AFB | | |
| | | | Culture | | |
| S122 | Positiuve | Achromobacter | Bacterial | n/a | BAL |
| | Colture | xylosaxidans | and Fungal | | |
| | (Bacteria) | | and AFB | | |
| | | | Culture | | |
| S123 | Positive | Cryptococcus | Bacterial | n/a | CSF |
| | Culture | gattii | and Fungal | | |
| | (Fungal) | | and AFB | | |
| | | | Culture | | |
| S124 | Positive | Coccidioides | Bacterial | 1 day prior: | CSF |
| | Culture | immitis | and Fungal | negative | |
| | (Fungal) | | and AFB | | |
| | | | Culture | | |
| S125 | Positive | Histoplasma | Bacterial | 3 days prior: | BAL |
| | Culture | capsulatum | and Fungal | 2x negative | |
| | (Fungal) | | and AFB | | |
| | | | Culture | | |
| S126 | Positive | Pneumnocytis | Bacterial | 3 days prior: | BAL |
| | Culture | jireoveci | and Fungal | 2x negative | |
| | (Fungal) | | and AFB | | |
| | | | Culture | | |
| S127 | Positive | Coccidioides | Bacterial | negative | CSF |
| | Culture | immitis | and Fungal | | |
| | (Fungal) | | and AFB | | |
| | | | Culture | | |
| S128 | Positive 16S | negative | Bacterial | 3-5 days prior: | Abscess |
| | | | and Fungal | negative | |
| | | | and AFB | | |
| | | | Culture | | |
| S129 | Positive | Staphylococcus | Bacterial | n/a | Abscess |
| | Culture | aureus | Culture | | |
| | (Bacteria) | | | | |
| S130 | Positive | Group B | Bacterial | n/a | Left Thigh |
| | Culture | Streptococcus | and Fungal | | Bursal |
| | (Bacteria) | | Culture | | Fluid |
| S131 | Positive | Klebsiella | Bacterial | 9, 8, 7 days prior: | CSF |
| | Culture | pneumoniae | and Fungal | Klebsiella | |
| | (Bacteria) | | Culture | pneumoniae | |
| S132 | Positive | Enterobacter | Bacterial | n/a | CSF |
| | Culture | aerogenes | Culture | | |
| | (Bacteria) | | | | |
| S133 | Positive | Staphylococcus | Bacterial | Same day, 2-3 | Iliopsosas |
| | Culture | aureus | Culture | days prior, 2 days | Collection |
| | (Bacteria) | | | later: positive for | Fluid |
| | | | | same organism | |
| S134 | Positive | Staphylococcus | Bacterial | Multiple positive | Left Iliac |
| | Culture | aureus | Culture | | Wing |
| | (Bacteria) | | | | Fluid |
| S135 | Positive | Staphylococcus | Bacterial | n/a | Abdominal |
| | Culture | aureus | Culture | | Fluid |
| | (Bacteria) | | | | Wall |
| S136 | Positive | Aspergillus | Bacterial | negative | BAL |
| | Culture | fumnigatus | and Fungal | | |
| | (Fungal) | | and AFB | | |
| | | | Culture | | |
| S137 | Positive | Klebsiella | Bacterial | n/a | Retrogastric |
| | Culture | pneumoniae | Culture | | Fluid |
| | (Bacteria) | | | | |
| S138 | Positive | Staphylococcus | Bacterial | n/a | Abdominal |
| | Culture | aureus | and Fungal | | Fluid |
| | (Bacteria) | | and AFB | | Wall |
| | | | Culture | | |
| S139 | Positive | Enterococcus | Bacterial | n/a | Peritoneal |
| | Culture | faecium | and AFB | | Fluid |
| | (Bacteria) | | Culture | | |
| S140 | Positive | Staphylococcus | Bacterial | n/a | Thoracic |
| | Culture | lugdunensis | Culture | | Spine |
| | (Bacteria) | | | | Seronia |
| S141 | Positive | Candida | Bacterial | negative | Perigastric |
| | Culture | parasilopsis | and Fungal | | Fluid |
| | (Fungal) | | and AFB | | |
| | | | Culture | | |
| S142 | Positive | Candida | Bacterial | n/a | Abscess |
| | Culture | tropicalis | Culture | | |
| | (Fungal) | | | | |
| S143 | Positive | Mycobacterium | Bacterial | n/a | FNA |
| | Culture | avium | and Fungal | | |
| | (Bacteria) | complex | and AFB | | |
| | | | Culture | | |
| S144 | Positive 16S | Mycobacterium | 16S, | n/a | Joint Fluid |
| | | avium | Bacterial. | | |
| | | complex | Fungal, | | |
| | | | and AFB | | |
| | | | Culture | | |
| S145 | Positive | Nocardia | Bacterial | negative | Pleural |
| | Culture | blacklockiae | and Fungal | | Fluid |
| | (Bacteria) | | and AFB | | |
| | | | Culture | | |
| S146 | Positive | Listeria | Bacterial | same day 2x | CSF |
| | Culture | monocytogenes | and Fungal | Listeria | |
| | (Bacteria) | | and AFB | monocytogenes | |
| | | | Culture | | |
| S147 | Positive | Staphylococcus | Bacterial | negative | Back |
| | Culture | aureus | Culture | | Fluid |
| | (Bacteria) | | | | |
| S148 | Positive | Staphylococcus | Bacterial | n/a | Breast |
| | Culture | aureus | Culture | | Fluid |
| | (Bacteria) | | | | |
| S149 | Positive | Enterococcus | Bacterial | negative | Peritoneal |
| | Culture | faecium, | Culture | | Fluid |
| | (Bacteria) | Enterococcus | | | |
| | | faecalis | | | |
| S150 | Positive | Staphylococcus | Bacterial | same day 2x | Synovial |
| | Culture | aureus | Culture | Staphylococcus | Fluid |
| | (Bacteria) | | | aureus; also | |
| | | | | positive the | |
| | | | | next day | |
| S151 | Positive 165 | Mycobacterium | Bacterial | negative | Peritoneal |
| | | tuberculosis | and Fungal | | Fluid |
| | | | and AFB | | |
| | | | Culture | | |
| S152 | Positive | Mycobacterium | Bacterial | negative | Peritoneal |
| | Culture | tuberculosis | and Fungal | | Fluid |
| | (Bacteria) | | and AFB | | |
| | | | Culture | | |
| S153 | Negative | negative | Bacterial | n/a | Peritoneal |
| | Control | | Culture | | Fluid |
| | (cause: | | | | |
| | cancer) | | | | |
| S154 | Negative | negative | Bacterial | n/a | Pleural |
| | Control | | Culture | | Fluid |
| | (cause: | | | | |
| | cancer) | | | | |
| S155 | Negative | negative | Bacterial | n/a | Pleural |
| | Control | | Culture | | Fluid |
| | (cause: | | | | |
| | cancer) | | | | |
| S156 | Negative | negative | Bacterial | n/a | Peritoneal |
| | Control | | and Fungal | | Fluid |
| | cause: | | Culture | | |
| | cancer) | | | | |
| S157 | Negative | negative | Bacterial | negative | Pleural |
| | Control | | and AFB | | Fluid |
| | (cause: | | Culture | | |
| | cancer) | | | | |
| S158 | Negative | negative | Bacterial | negative | Peritoneal |
| | Control | | and Fungal | | Fluid |
| | (cause: | | and AFB | | |
| | cancer) | | Culture | | |
| S159 | Negative | negative | Bacterial | negative | Pleural |
| | Control | | and Fungal | | Fluid |
| | (cause: | | and AFB | | |
| | cancer) | | Culture | | |
| S160 | Negative | negative | Bacterial | negative | Pleural |
| | Control | | and Fungal | | Fluid |
| | (cause: | | and AFB | | |
| | cancer) | | Culture | | |
| S161 | Negative | negative | Bacterial | n/a | Pleural |
| | Control | | Culture | | Fluid |
| | (cause: | | | | |
| | cancer) | | | | |
| S162 | Negative | negative | Bacterial | n/a | Pleural |
| | Control | | Culture | | Fluid |
| | (cause: | | | | |
| | cancer) | | | | |
| S163 | Negative | negative | Bacterial | n/a | Pleural |
| | Control | | and Fungal | | Fluid |
| | (cause: | | Culture | | |
| | cancer) | | | | |
| S164 | Negative | negative | Bacterial | n/a | Pleural |
| | Control | | Culture | | Fluid |
| | (cause: | | | | |
| | cancer) | | | | |
| S165 | Negative | negative | Bacterial | n/a | Pleural |
| | Control | | and Fungal | | Fluid |
| | (cause: | | Culture | | |
| | Cancer) | | | | |
| S166 | Negative | negative | Bacterial | n/a | Peritoneal |
| | Control | | Culture | | Fluid |
| | (cause: | | | | |
| | cancer) | | | | |
| S167 | Negative | negative | Bacterial | Oct. 24, 2018: | Pleural |
| | Control | | Culture | Escherichia coli | Fluid |
| | (cause: | | | | |
| | cancer) | | | | |
| S168 | Negative | negative | Bacterial | 5/29: | Peritoneal |
| | Control | | Culture | Staphylococcus | Fluid |
| | (cause: | | | epidermidis | |
| | cancer) | | | | |
| S169 | Negative | negative | Bacterial | n/a | CSF |
| | Control | | and Fungal | | |
| | (cause: | | Culture | | |
| | cancer) | | | | |
| S170 | Negative | negative | Bacterial | n/a | CSF |
| | Control | | and Fungal | | |
| | (cause: | | and AFB | | |
| | cancer) | | Culture | | |
| S171 | Negative | negative | Bacterial | n/a | CSF |
| | Control | | and Fungal | | |
| | (cause: | | and AFB | | |
| | cancer) | | Culture | | |
| S172 | Negative | negative | Bacterial | negative | CSF |
| | Control | | Culture | | |
| | (cause: | | | | |
| | cancer) | | | | |
| S173 | Negative | negative | Bacterial | negative | CSF |
| | Control | | Culture | | |
| | (cause: | | | | |
| | cancer) | | | | |
| S174 | Negative | negative | Bacterial | negative | CSF |
| | Control | | and Fungal | | |
| | (cause: | | and AFB | | |
| | cancer) | | Culture | | |
| S175 | Negative | negative | Bacterial | 14 days later: | CSF |
| | Control | | and Fungal | Enterobacter | |
| | (cause: | | Culture | aerogenes | |
| | cancer) | | | | |
| S176 | Suspected | negative but | Bacterial | n/a | BAL |
| | culture FN: | highly | and Fungal | | |
| | respiratory | probable | and AFB | | |
| | fungal | infection | Culture | | |
| | infection | | | | |
| S177 | Suspected | negative but | Bacterial | 9 days later: | CSF |
| | culture FN: | highly | and Fungal | several | |
| | CNS infection | probable | and AFB | | |
| | confirmed by | infection | Culture | | |
| | culture from | | | | |
| | brain biopsy | | | | |
| S178 | Suspected | negative but | Bacterial | negative | Pleural |
| | culture FN: | highly | and Fungal | | Fluid |
| | Cryptococcus | probable | and AFB | | |
| | pneumonia | infection | Culture | | |
| S179 | Suspected | negative but | Bacterial | negative | CSF |
| | culture FN: | highly | and Fungal | | |
| | neurosyphilis | probable | and AFB | | |
| | | infection | Culture | | |
| S180 | Suspected | negative but | Bacterial | n/a | Pleural |
| | culture FN: | highly | and Fungal | | Fluid |
| | tuberculous | probable | and AFB | | |
| | infection | infection | Culture | | |
| S181 | Suspected | negative but | Bacterial | n/a | CSF |
| | culture FN: | highly | and Fungal | | |
| | CNS infection | probable | and AFB | | |
| | confirmed by | infection | Culture | | |
| | later culture | | | | |
| S182 | Suspected | negative but | Bacterial | n/a | CSF |
| | culture FN: | highly | and Fungal | | |
| | Sporothrix | probable | and AFB | | |
| | infection by | infection | Culture | | |
| | serology | | | | |

| | | Organism | | |
|---|---|---|---|---|
| | Organism | detected by | nRPM | |
| | detected by | Nacopore at | snoopore | Sequencing to |
| Sample | Illumina over | Validation | (1st org if | Detection |
| # | threshold | threshold | polymicrobial) | Time (mins) |
| S1 | Staphylococcus | Staphylococcus | 106.33 | 128 |
| | aureus | aureus | | |
| S2 | Enterobacter | Klebsiella | 27155.11 | 21 |
| | aerogenes | aerogenes | | |
| S3 | Streptococcus | Streptococcus | 197.81 | 23 |
| | agalactiae | agalactiae | | |
| S4 | Candida | Candida | 0.54 | 30 |
| | parapsilosis | parapsilosis | | |
| S5 | Saccharomyces | <negative> | | 110 |
| | cerevisiae, off | | | |
| | species hits | | | |
| | related to | | | |
| | Saccharomyces | | | |
| | cerevisiae | | | |
| | (confirmed | | | |
| | with BLAST) | | | |
| S6 | Pseudomonas | Pseudomonas | 340.11 | 22 |
| | aeruginosa | aeruginosa | | |
| S7 | <negative> | <negative> | | 80 |
| S8 | <negative> | Pseudomonas | 236.16 | 65 |
| | | aeruginosa | | |
| S9 | Staphylococcuss | Staphylococcuss | 1114.42 | 30 |
| | aureus | aureus | | |
| S10 | Haemophilus | Haemophilus | 8483.10 | 21 |
| | influenzae | influenzae | | |
| | Rothia | | | |
| | dentocariosa | | | |
| S11 | <negative> | Staphylococcus | 4.12 | 65 |
| | | aureus | | |
| S12 | <negative> | <negative> | | 90 |
| S13 | <negative> | Staphylococcus | 1.25 | 92 |
| | | aureus | | |
| S14 | Pseudomonas | Pseudomon | 252.77 | 23 |
| | aeruginosa | deruginosa | | |
| S15 | Staphylococcus | Staphylococcus | 0.60 | 320 |
| | aureus | aureus | | |
| S16 | <negative> | <negative> | | 90 |
| S17 | Serratia sp. | Serratia | 42.71 | 40 |
| | SCBI, | marcescens, | | |
| | Enterococcus | Enterococcus | | |
| | faecium | faecium | | |
| S18 | Staphylococcus | Staphylococcus | 3130.95 | 21 |
| | epidermidis | epidermidis | | |
| S19 | Serratia sp. | Serratia | 8.72 | 30 |
| | SCBI, | marcescens | | |
| | Enterococcus | Enterococcus | | |
| | faecium | faecium | | |
| S20 | Enterococcus | Enterococcus | 93.15 | 21 |
| | faecium | faecium, | | |
| | | Stenotrophomonas | | |
| | | maltophilia | | |
| S21 | Enterococcus | Enterococcus | 2.87 | 50 |
| | faecium | faecium | | |
| S22 | Candida | Candida | 236.16 | 50 |
| | albicans, | glabrata, | | |
| | Candida | Candida | | |
| | glabrata | albicans | | |
| S23 | Staphylococcus | <negative> | | 80 |
| | aureus | | | |
| S24 | <negative> | <negative> | | 80 |
| S25 | <negative> | <negative> | | 75 |
| S26 | Enterococcus | Enterococcus | 14.86 | 90 |
| | faecium | faecium | | |
| S27 | Candida | Candida | 4.72 | 40 |
| | glabrata, | glabrata | | |
| | Candida | | | |
| | albicans | | | |
| S28 | Escherichia | Escherichia | 1283.49 | 21 |
| | coli | coli | | |
| S29 | Enterobacter | Enterobacter | 40.66 | 50 |
| | cloacae | kobei | | |
| S30 | Staphylococcus | Staphylococcus | 84.20 | 29 |
| | aureus | aureus | | |
| S31 | Klebsiella | Klebsiella | 12.79 | 22 |
| | pneumoniae | pneumoniae | | |
| S32 | Staphylococcus | Staphylococ | 16.31 | 50 |
| | aureus | | | |
| S33 | <negative> | <negative> | | 90 |
| S34 | Streptococcus | Streptococcus | 75.23 | 40 |
| | constellatus, | anginosus | | |
| | Streptococcus | group | | |
| | anginosus | | | |
| | Streptococcus | | | |
| | intermedius, | | | |
| | Parvimonas | | | |
| | micra | | | |
| S35 | <negative> | <negative> | | 80 |
| S36 | Staphylococcus | <negative> | | 72 |
| | aureus | | | |
| S37 | Enterococcus | Enterococcus | 1461.31 | 21 |
| | faecalis, | faecium, | | |
| | Enterococcus | Enterococcus | | |
| | faecalis, | faecalis | | |
| | Prevotella | | | |
| | malaningenica, | | | |
| | Lactobacillus | | | |
| | gasseri, | | | |
| | Campylobacter | | | |
| | curvus, | | | |
| | Peptoclostridiam | | | |
| | difficile, | | | |
| | Campylobacter | | | |
| | conrisus | | | |
| S38 | Staphylococcus | <negative> | | 80 |
| | aureus | | | |
| S39 | <negative> | <negative> | | 90 |
| S40 | <negative> | Achromobacter | 0.95 | 80 |
| | | xylosoidans | | |
| S41 | Streptococcus | Streptococcus | 85.99 | 25 |
| | pyogenes | pyogenes | | |
| S42 | Escherichia | Escherichia | 237.70 | 25 |
| | coli | coli | | |
| S43 | Staphylococcus | Staphylococcus | 21.55 | 32 |
| | aureus | aureus | | |
| S44 | Staphylococcus | Staphylococcus | 7.86 | 70 |
| | aureus | aureus | | |
| S45 | Staphylococcus | Staphylococcus | 79.77 | 32 |
| | aureus | aureus | | |
| S46 | Staphylococcus | Staphylococcus | 19.69 | 26 |
| | aureus | aureus | | |
| S47 | Klebsiella | Klebsiella | 950.19 | 22 |
| | pneumaniae, | pneumaniae, | | |
| | Excherichia | Excherichia | | |
| | coli | coli | | |
| S48 | <negative> | <negative> | | 80 |
| S49 | Enterococcus | Enterococcus | 332.58 | 23 |
| | faecium, | faecium, | | |
| | Candida | Candida | | |
| | albicans | albicans | | |
| S50 | Klebsiella | Klebsiella | 2.32 | 35 |
| | pneumoniae | pneumoniae | | |
| S51 | Streptococcus | <negative> | | 90 |
| | pneumoniae, | | | |
| | Aspergillus | | | |
| | fumigatus, | | | |
| | Prevotella | | | |
| | melaminogenica | | | |
| S52 | Staphylococcus | Staphylococcus | 15.89 | 50 |
| | aureus | aureus | | |
| S53 | Citrobacter | Klebsiella | 37.36 | 50 |
| | freundii, | pneumaniae, | | |
| | Klebsiella | Citrobacter | | |
| | pneumoniae, | freundii | | |
| | Salmonella | | | |
| | enterica, | | | |
| | Bacteroides | | | |
| | xylanisolvens, | | | |
| | Bacteroides | | | |
| | thetaiotaomicron | | | |
| S54 | Escherichia | Escherichia | 615.27 | 22 |
| | coli | coli | | |
| S55 | <negative> | Staphylococcus | 0.45 | 130 |
| | | aureus | | |
| S56 | Klebsiella | Klebsiella | 2022.59 | 21 |
| | pneumoniae, | pneumoniae | | |
| | Enterococcus | | | |
| | faecium, | | | |
| | gamma | | | |
| | proteobacterium | | | |
| | HdN1 | | | |
| S57 | Klebsiella | Klebsiella | 2.07 | 110 |
| | pneumoniae | pneumoniae | | |
| S58 | Aspergillus | Aspergillus | 4.16 | 50 |
| | fumigatus | fumigatus | | |
| S59 | Pseudomonas | Pseudomonas | 1619.76 | 22 |
| | aeruginosa, | aeruginosa, | | |
| | Staphylococcus | Candida | | |
| | epidermidis, | glabrata, | | |
| | Candida | Staphylococcus | | |
| | glabrata, | epidermidis, | | |
| | Pichia kluyveri, | Neisseria | | |
| | Lactococcus | sicca, | | |
| | lactis, | Leuconostoc | | |
| | Campylobacter | citreum | | |
| | concisus, | | | |
| | Lactobacillus | | | |
| | acidophilus, | | | |
| | Veillonella | | | |
| | parvuila | | | |
| S60 | Pseudomonas | Pseudomonas | 576.50 | 22 |
| | aeruginosa | aeruginosa | | |
| S61 | Staphylococcus | Staphylococcus | 9.35 | 30 |
| | lugdimensis | lugdimensis | | |
| S62 | Escherichia | Escherichia | 54.07 | 21 |
| | coli | coli | | |
| S63 | <negative> | <negative> | | 40 |
| S64 | Mycoplasma | Mycoplasma | 33.14 | 23 |
| | hominis | hominis | | |
| S65 | Streptococcus | Streptococcus | 6.35 | 35 |
| | pyogenes | pyogenes | | |
| S66 | <negative> | <negative> | | 88 |
| S67 | <negative> | <negative> | | 88 |
| S68 | <negative> | <negative> | | 88 |
| S69 | Mycobacterium | Mycobacterium | 2.87 | 200 |
| | tuberculosis | tuberculosis | | |
| S70 | Staphylococcus | Staphylococcus | 5.42 | 56 |
| | aureus | aureus | | |
| S71 | Staphylococcus | Staphylococcus | 3.86 | 21 |
| | aureus | aureus | | |
| S72 | Candida | <negative> | | 110 |
| | albicans | | | |
| S73 | Staphylococcus | Staphylococcus | 2983.43 | 21 |
| | aureus | aureus | | |
| S74 | Enterococcus | Enterococcus | 1502.62 | 65 |
| | faecalis, | faecalis, | | |
| | Escherichia | Escherichia | | |
| | coli, | coli, | | |
| | Staphylococcus | Staphylococcus | | |
| | aureus, | aureus | | |
| | Streptococcus | | | |
| | mitis, | | | |
| | Bifidobacterium | | | |
| | breve, | | | |
| | Peptoclostridsum | | | |
| | difficile | | | |
| S75 | Streptococcus | Streptococcus | 59.03 | 26 |
| | mitis group | gordonii | | |
| S76 | Escherichia | Escherichia | 372.95 | 21 |
| | coli | coli | | |
| S77 | <negative> | <negative> | | 80 |
| S78 | Escherichia | Escherichia | 525.95 | 21 |
| | coli | coli | | |
| | | Corynebacterium | | |
| | | striatum | | |
| S79 | Coccidioides | Coccidioides | 30.07 | 23 |
| | immitis | immitis | | |
| S80 | Coccidioides | Coccidioides | 0.69 | 80 |
| | immitis | immitis | | |
| S81 | Streptococcus | Streptococcus | 129.99 | 23 |
| | pyogenes | pyogenes | | |
| S82 | <negative> | <negative> | | 80 |
| S83 | Aspergillus | Aspergillus | 0.72 | 60 |
| | terreus, | terreus, | | |
| | Aspergillus | Aspergillus | | |
| | fumigatus | fumigatus | | |
| S84 | <negative> | <negative> | | 80 |
| S85 | Streptococcus | Streptococcus | 4.10 | 65 |
| | pyogenes, | pyogenes | | |
| | Talaromyces | | | |
| | marneffei | | | |
| S86 | <negative> | Not tested | n/a | |
| S87 | Finegoldia | Not tested | n/a | |
| | magna, | | | |
| | Bartonella | | | |
| | henselae | | | |
| S88 | Klebsiella | Klebsiella | 21.26 | 23 |
| | aerogenes | aerogenes | | |
| S89 | Polymicrobial | Not tested | n/a | |
| | anaerobes | | | |
| S90 | Streptococcus | Not tested | n/a | |
| | pneumoniae | | | |
| S91 | <negative> | Not tested | n/a | |
| S92 | Aspergillus | Not tested | n/a | |
| | fumigatus | | | |
| S93 | <negative> | — | — | — |
| S94 | Coccidioides | — | — | — |
| | immitis | | | |
| S95 | Staphylococcus | — | — | — |
| | aureus | | | |
| S96 | Staphylococcus | — | — | — |
| | aureus, | | | |
| | Enterococcus | | | |
| | faecalis | | | |
| S97 | Candida | — | — | — |
| | glatrata | | | |
| S98 | Streptococcus | — | — | — |
| | pyogenes | | | |
| S99 | Streptococcus | — | — | — |
| | pneumoniae | | | |
| S100 | Staphylococcus | — | — | — |
| | epidermis | | | |
| S101 | Candida | — | — | — |
| | parapsilosis, | | | |
| | Candida | | | |
| | albicans | | | |
| S102 | Staphylococcus | — | — | — |
| | aureus | | | |
| S103 | Escherichia | — | — | — |
| | coli | | | |
| S104 | Candida | — | — | — |
| | glabrata | | | |
| S105 | Cryptococcus | — | — | — |
| | neoformans | | | |
| S106 | Candida | — | — | — |
| | parasilopsis | | | |
| S107 | Candida | — | — | — |
| | parasilopsis | | | |
| S108 | Candida | — | — | — |
| | parasilopsis | | | |
| S109 | Cryptococcus | — | — | — |
| | neoformans | | | |
| S110 | Pseudomonas | — | — | — |
| | aeruginosa | | | |
| S111 | Staphylococcus | — | — | — |
| | aureus | | | |
| S112 | Enterococcus | — | — | — |
| | faecium | | | |
| S113 | Staphylococcus | — | — | — |
| | aureus | | | |
| S114 | Coccidioides | — | — | — |
| | immitis, | | | |
| | Methylobacterium | | | |
| | radiotolerans, | | | |
| | Methylobacterium | | | |
| | extarquens, | | | |
| | Burkholderia | | | |
| | gladioli, | | | |
| | Methylobacterium | | | |
| | populi, | | | |
| | Sphingomonas | | | |
| | taxi | | | |
| S115 | Streptococcus | — | — | — |
| | pyogenes, | | | |
| | Corynebacterium | | | |
| | diphtheriae | | | |
| S116 | Cryptococcus | — | — | — |
| | neoformans, | | | |
| | Streptococcus | | | |
| | parasanguinis | | | |
| S117 | Salmonella | — | — | — |
| | enterica | | | |
| S118 | Mycobacterium | — | — | — |
| | tuberculosis | | | |
| | complex | | | |
| S119 | Escherichia | — | — | — |
| | coli | | | |
| S120 | Enterobacter | — | — | — |
| | cloacae, | | | |
| | Candida | | | |
| | albicans | | | |
| S121 | Coccidioides | — | — | — |
| | immitis | | | |
| S122 | Achromobacter | — | — | — |
| | xylosoxidans, | | | |
| | Staphylococcus | | | |
| | epidermidis, | | | |
| | Pseudomonas | | | |
| | pseudoalcoliganes | | | |
| S123 | Cyptococcus | — | — | — |
| | gattii | | | |
| S124 | Coccidioides | — | — | — |
| | immitis | | | |
| S125 | <negative> | — | — | — |
| S126 | Pneumocystis | — | — | — |
| | jirovecii | | | |
| S127 | Coccidioides | — | — | — |
| | immitis | | | |
| S128 | Aggregatibacter | — | — | — |
| | aphrophilus | | | |
| S129 | Staphylococcus | — | — | — |
| | aureus | | | |
| S130 | Streptococcus | — | — | — |
| | agalactiae | | | |
| S131 | Klebsiella | — | — | — |
| | pneumoniae | | | |
| S132 | Enterobacter | — | — | — |
| | aerogenes | | | |
| S133 | Staphylococcus | — | — | — |
| | aureus, | | | |
| | Bacillus | | | |
| | thuringiensis | | | |
| S134 | Staphylococcus | — | — | — |
| | aureus | | | |
| S135 | Staphylococcus | — | — | — |
| | aureus | | | |
| S136 | Aspergillus | — | — | — |
| | fumigatus | | | |
| S137 | Klebsiella | — | — | — |
| | pneumoniae, | | | |
| | Sodatis sp. | | | |
| | Veilionella | | | |
| | parvula | | | |
| S138 | Staphylococcus | — | — | — |
| | aureus | | | |
| S139 | Enterococcus | — | — | — |
| | faecium, | | | |
| | Bordetella | | | |
| | petrii | | | |
| S140 | Staphylococcus | — | — | — |
| | lugdunensis | | | |
| S141 | <negative> | — | — | — |
| S142 | Candida | — | — | — |
| | tropicalis | | | |
| S143 | Mycobacterium | — | — | — |
| | avium | | | |
| S144 | <negative> | — | — | — |
| S145 | <negative> | — | — | — |
| S146 | Listeria | — | — | — |
| | monocytogenes | | | |
| S147 | Staphylococcus | — | — | — |
| | aureus | | | |
| S148 | Staphylococcus | — | — | — |
| | aureus | | | |
| S149 | Enterococcus | — | — | — |
| | faecium, | | | |
| | Enterococcus | | | |
| | faecalis | | | |
| | Peptoclostridium | | | |
| | difficile | | | |
| S150 | Staphylococcus | — | — | — |
| | aureus | | | |
| S151 | <negative> | — | — | — |
| S152 | <negative> | — | — | — |
| S153 | <negative> | — | — | — |
| S154 | <negative> | — | — | — |
| S155 | <negative> | — | — | — |
| S156 | <negative> | — | — | — |
| S157 | <negative> | — | — | — |
| S158 | <negative> | — | — | — |
| S159 | <negative> | — | — | — |
| S160 | <negative> | — | — | — |
| S161 | <negative> | — | — | — |
| S162 | <negative> | — | — | — |
| S163 | <negative> | — | — | — |
| S164 | <negative> | — | — | — |
| S165 | <negative> | — | — | — |
| S166 | <negative> | — | — | — |
| S167 | Propionibacterium | — | — | — |
| | acnes | | | |
| S168 | <negative> | — | — | — |
| S169 | <negative> | — | — | — |
| S170 | <negative> | — | — | — |
| S171 | <negative> | — | — | — |
| S172 | <negative> | — | — | — |
| S173 | <negative> | — | — | — |
| S174 | <negative> | — | — | — |
| S175 | <negative> | — | — | — |
| S176 | Streptococcus | — | — | — |
| | sp. VT 162, | | | |
| | Streptococcus | | | |
| | oralis, | | | |
| | Veillonella | | | |
| | parvula, Rothia | | | |
| | muculaginosa | | | |
| S177 | <negative> | — | — | — |
| S178 | <negative> | — | — | — |
| S179 | <negative> | — | — | — |
| S180 | <negative> | — | — | — |
| S181 | <negative> | — | — | — |
| S182 | <negative> | — | — | — |

DNA extraction. Samples were processed in a blinded fashion In a CLIA (Clinical Laboratory Improvement Amendments)-certified clinical microbiology laboratory with physically separate pre- and post-PCR rooms. Cells were first removed through centrifugation to minimize host background. 400 μL of body fluid supernatant or plasma then underwent total nucleic acid extraction to 60 μL extract using the EZ1 Advanced XL BioRobot and EZ1 Virus Mini Kit v2.0 (QIAGEN) according to the manufacturer's instructions.

Library preparation and PCR amplification. Library preparation was performed using the NEBNext Ultra II DNA Library Prep Kit (New England Biolabs), with the use of 25 μL of extracted DNA input and half of the reagent volumes suggested by the manufacturer's protocol. Briefly, extracted DNA from most samples was quantified on a NanoDrop spectrophotometer (ThermoFisher) and diluted to 10-100 ng of input as recommended by the manufacturer. Plasma or CSF DNA was not quantified or diluted as typical input concentrations of <10 ng/μL could not be reliably detected using a spectrophotometer. The DNA was then end-repaired, ligated with the NEBNext Adapter (0.6 μM final concentration) to enrich for short-fragment pathogen DNA (100-800 nt) relative to residual human genomic DNA (>1 kb), and cleaned using AMPure beads. In addition to the initial manual preparation of 17 samples, an automated protocol using the epMotion 5075 liquid handler (Eppendorf) was used to process the remaining 165 samples, with 16-48 samples batch-processed per run.

PCR amplification was performed using a 40 μL mix consisting of adapter-ligated DNA, premixed custom index primers at 3 μM final concentration (see Table 2), and a quantitative PCR master mix (KAPA RT-kit, KK2702, Roche). DNA amplification was performed to saturation of the fluorescent signal on a qPCR thermocycler (Lightcycler 480, Roche) using the following PCR conditions: initiation at 98° C.×45 s, then 24 cycles of 98° C.×15 s/63° C.×30 s/72° C.×90 s, and a final extension step of 72° C.×60 s. Ct values were continually monitored until the libraries were fully amplified to saturation. Final DNA libraries were cleaned up using Ampure beads (Beckman) at a 0.9×volumetric ratio and eluted in 30 μL EB buffer (Qiagen).

TABLE 2

| | SEQ ID | |
|---|---|---|
| Index | NO | i7/P7 primer |
| 1 | 5 | CAAGCAGAAGACGGCATACGAGAT CTCCGTATCTTCTCCATTTGTTGTGCAGAAATGGCTC GTGACTGGAGTTCAGACGTGTGCTCTTCCGATC*T |
| 2 | 6 | CAAGCAGAAGACGGCATACGAGAT GAGACTCTATACCTCCTCCTCTATACGTTCGCTTCAT GTGACTGGAGTTCAGACGTGTGCTCTTCCGATC*T |
| 3 | 7 | CAAGCAGAAGACGGCATACGAGAT ACCGTGGAGTCATAAGCTTGACCTCGCCACATGTCTG GTGACTGGAGTTCAGACGTGTGCTCTTCCGATC*T |
| 4 | 8 | CAAGCAGAAGACGGCATACGAGAT CCGGACTGATGACCGGATTAAGTTCGCAGTCACCGAA GTGACTGGAGTTCAGACGTGTGCTCTTCCGATC*T |
| 5 | 9 | CAAGCAGAAGACGGCATACGAGAT GCTCTAGCCGTCACTCTTTATCCTCACACTACTGGCT GTGACTGGAGTTCAGACGTGTGCTCTTCCGATC*T |
| 6 | 10 | CAAGCAGAAGACGGCATACGAGAT CATCTGTTCTCGTTACACAGAGCTGCCAAGTACAGTG GTGACTGGAGTTCAGACGTGTGCTCTTCCGATC*T |
| 7 | 11 | CAAGCAGAAGACGGCATACGAGAT CCGTTCTCTTGAGCGCATTATAGAAGCGCCAAGATCG GTGACTGGAGTTCAGACGTGTGCTCTTCCGATC*T |
| 8 | 12 | CAAGCAGAAGACGGCATACGAGAT ATCGTGGTCGCTTACCGTTGTCAAGGACAAGCTGATG GTGACTGGAGTTCAGACGTGTGCTCTTCCGATC*T |
| 9 | 13 | CAAGCAGAAGACGGCATACGAGAT AGAGCAATGACGATATGTTCTTCGGCATGGTAGTAGC GTGACTGGAGTTCAGACGTGTGCTCTTCCGATC*T |
| 10 | 14 | CAAGCAGAAGACGGCATACGAGAT GTCGGTATCTTATGTGCAGCTGTTCGACCGGTGTACA GTGACTGGAGTTCAGACGTGTGCTCTTCCGATC*T |
| 11 | 15 | CAAGCAGAAGACGGCATACGAGAT GTGACAACTGAGTGACTTTATGCTGCCGGCTCTCAAC GTGACTGGAGTTCAGACGTGTGCTCTTCCGATC*T |
| 12 | 16 | CAAGCAGAAGACGGCATACGAGAT GAACGATTCCAACGTAATTGTGTTGTCCTCAAGGAGT GTGACTGGAGTTCAGACGTGTGCTCTTCCGATC*T |
| 13 | 17 | CAAGCAGAAGACGGCATACGAGAT GGTTCGCAGGCAGGTCACAACACCGTTCTTTACGGAG GTGACTGGAGTTCAGACGTGTGCTCTTCCGATC*T |
| 14 | 18 | CAAGCAGAAGACGGCATACGAGAT TGTTCTCCCAATTGTAGTTGCTCCACATATCTGTGCA GTGACTGGAGTTCAGACGTGTGCTCTTCCGATC*T |
| 15 | 19 | CAAGCAGAAGACGGCATACGAGAT ATGGCCGTCAGTTGTGTAACTGTGACCTCTCTGAGGT GTGACTGGAGTTCAGACGTGTGCTCTTCCGATC*T |
| 16 | 20 | CAAGCAGAAGACGGCATACGAGAT TAAGCGTCCATGATTGATGCTAATGTTCCCTCTACCT GTGACTGGAGTTCAGACGTGTGCTCTTCCGATC*T |
| 17 | 21 | CAAGCAGAAGACGGCATACGAGAT TAACTGTCTTAGGCTAATTCTGGACTAGCATGTTCGC GTGACTGGAGTTCAGACGTGTGCTCTTCCGATC*T |
| 18 | 22 | CAAGCAGAAGACGGCATACGAGAT TGGCCACGGTCATTGTACAGGTACCGCATAGTCCTAG GTGACTGGAGTTCAGACGTGTGCTCTTCCGATC*T |
| 19 | 23 | CAAGCAGAAGACGGCATACGAGAT TAGCGAAAGATGCCGACGAATAGCTTGCGGGTCAGAT GTGACTGGAGTTCAGACGTGTGCTCTTCCGATC*T |
| 20 | 24 | CAAGCAGAAGACGGCATACGAGAT CCATACGGCCTGGATCACCAATATGGAGCCGTCCATA GTGACTGGAGTTCAGACGTGTGCTCTTCCGATC*T |
| 21 | 25 | CAAGCAGAAGACGGCATACGAGAT ACGTCTCATGCCACCTGTTGGCCATGCAGTTCTCTGG GTGACTGGAGTTCAGACGTGTGCTCTTCCGATC*T |
| 22 | 26 | CAAGCAGAAGACGGCATACGAGAT CAGAGACATACATGGCTCCAAGTTCAGGCGTCCTTCT GTGACTGGAGTTCAGACGTGTGCTCTTCCGATC*T |
| 23 | 27 | CAAGCAGAAGACGGCATACGAGAT CGAGCTTCTCGACCATACCACATCGACATTTCCGCAA GTGACTGGAGTTCAGACGTGTGCTCTTCCGATC*T |
| 24 | 28 | CAAGCAGAAGACGGCATACGAGAT AACCGAGGATAGCACCGTACCATCCATCTAGGATACC GTGACTGGAGTTCAGACGTGTGCTCTTCCGATC*T |
| 25 | 29 | CAAGCAGAAGACGGCATACGAGAT TATGGCGGCTGCGATTCTGGAGATATGTGCGCTATGT GTGACTGGAGTTCAGACGTGTGCTCTTCCGATC*T |
| 26 | 30 | CAAGCAGAAGACGGCATACGAGAT GAGAGAGTGACCAAGTACCACTAGTTAAGCAGCTACT GTGACTGGAGTTCAGACGTGTGCTCTTCCGATC*T |
| 27 | 31 | CAAGCAGAAGACGGCATACGAGAT CTGGTCCAGCTTCTCTAACAAGTTGGTTCGAGAAGTC GTGACTGGAGTTCAGACGTGTGCTCTTCCGATC*T |
| 28 | 32 | CAAGCAGAAGACGGCATACGAGAT GTCACACGCTCACCGTTTGGACCTGTTGGTTCGCGTA GTGACTGGAGTTCAGACGTGTGCTCTTCCGATC*T |
| 29 | 33 | CAAGCAGAAGACGGCATACGAGAT GGTCCTATCGTGTCTAATTCCGCCTTGGATTGAGGCA GTGACTGGAGTTCAGACGTGTGCTCTTCCGATC*T |
| 30 | 34 | CAAGCAGAAGACGGCATACGAGAT AGTTCACTCCAGATCTGTTGCGCACCTTGCTTCACAG GTGACTGGAGTTCAGACGTGTGCTCTTCCGATC*T |
| 31 | 35 | CAAGCAGAAGACGGCATACGAGAT ACGGTGCTGTAGTCAGCTTAGCACTTCGGTACCACCT GTGACTGGAGTTCAGACGTGTGCTCTTCCGATC*T |
| 32 | 36 | CAAGCAGAAGACGGCATACGAGAT AAGATTCATACGACAGTTAAGCTAGAGACTTCCTCGT GTGACTGGAGTTCAGACGTGTGCTCTTCCGATC*T |
| 33 | 37 | CAAGCAGAAGACGGCATACGAGAT GACGTGACGGCTGCAATTTGAGCGTTGTTTACCTCTC GTGACTGGAGTTCAGACGTGTGCTCTTCCGATC*T |
| 34 | 38 | CAAGCAGAAGACGGCATACGAGAT CAACTACTCGGATTGAGTTGTACGTCCGCCGAGTTGT GTGACTGGAGTTCAGACGTGTGCTCTTCCGATC*T |
| 35 | 39 | CAAGCAGAAGACGGCATACGAGAT ACGGCAGTGGTACAACTTTGGTAGAGGTTGGTGGATT GTGACTGGAGTTCAGACGTGTGCTCTTCCGATC*T |
| 36 | 40 | CAAGCAGAAGACGGCATACGAGAT CGATTCTGCCGTGAACTTTCGACTATCCTGATGGAGA GTGACTGGAGTTCAGACGTGTGCTCTTCCGATC*T |
| 37 | 41 | CAAGCAGAAGACGGCATACGAGAT GTCGTAGACGGACCTGTTGAATGCCTCAACGAAGGTT GTGACTGGAGTTCAGACGTGTGCTCTTCCGATC*T |
| 38 | 42 | CAAGCAGAAGACGGCATACGAGAT GTAACTGAGCAACTGCCTCGAATTCCAAGTGCGGTAA GTGACTGGAGTTCAGACGTGTGCTCTTCCGATC*T |
| 39 | 43 | CAAGCAGAAGACGGCATACGAGAT TGTCCAACTAATGCTCTTTGTAGCCAATGTCACTTCG GTGACTGGAGTTCAGACGTGTGCTCTTCCGATC*T |
| 40 | 44 | CAAGCAGAAGACGGCATACGAGAT TTCGGACGGTTCATTAGTTCACAAGCGGATGAAGACC GTGACTGGAGTTCAGACGTGTGCTCTTCCGATC*T |
| 41 | 45 | CAAGCAGAAGACGGCATACGAGAT GGTTAAGCATGCCTCTATTCCTGATCTGACAGCATCA GTGACTGGAGTTCAGACGTGTGCTCTTCCGATC*T |
| 42 | 46 | CAAGCAGAAGACGGCATACGAGAT AATGTACTGTGACTCTTTACCAGAGACCGCCTTAACG GTGACTGGAGTTCAGACGTGTGCTCTTCCGATC*T |
| 43 | 47 | CAAGCAGAAGACGGCATACGAGAT GGTTACCATGCACCAATTTGATGGAACCTGTCTCACA GTGACTGGAGTTCAGACGTGTGCTCTTCCGATC*T |
| 44 | 48 | CAAGCAGAAGACGGCATACGAGAT TCGTAGAACCTCCACAGTTGTCTCTTGTGACACCTTC GTGACTGGAGTTCAGACGTGTGCTCTTCCGATC*T |
| 45 | 49 | CAAGCAGAAGACGGCATACGAGAT GCATGGAACACCTCGTTTTCTCAGGTTGGCTGCACTA GTGACTGGAGTTCAGACGTGTGCTCTTCCGATC*T |
| 46 | 50 | CAAGCAGAAGACGGCATACGAGAT AAGATCGTATAGCGCTCTTCGGTGTTCCGGCTAGGAA GTGACTGGAGTTCAGACGTGTGCTCTTCCGATC*T |
| 47 | 51 | CAAGCAGAAGACGGCATACGAGAT ACGGATTCCTAACAGGTAAGCGTAAGCGGTTGTTGCC GTGACTGGAGTTCAGACGTGTGCTCTTCCGATC*T |
| 48 | 52 | CAAGCAGAAGACGGCATACGAGAT ATCCATATGGCGTTGGACCGTCGTTATCAGCCGATAT GTGACTGGAGTTCAGACGTGTGCTCTTCCGATC*T |
| 49 | 53 | CAAGCAGAAGACGGCATACGAGAT TAACCATTCGCTTCATTTTCCGAACAGGTCCGACTTA GTGACTGGAGTTCAGACGTGTGCTCTTCCGATC*T |
| 50 | 54 | CAAGCAGAAGACGGCATACGAGAT GAGTGTTTGCCTATGTTTACGAACGCCTTTTCCGTTC GTGACTGGAGTTCAGACGTGTGCTCTTCCGATC*T |
| 51 | 55 | CAAGCAGAAGACGGCATACGAGAT TACTAACCCGTTCTTCGTTCTGGTTGACATCGGAGAA GTGACTGGAGTTCAGACGTGTGCTCTTCCGATC*T |
| 52 | 56 | CAAGCAGAAGACGGCATACGAGAT TATACAATAGTGATTGCCCGAGGATCGAACCAACCAA GTGACTGGAGTTCAGACGTGTGCTCTTCCGATC*T |
| 53 | 57 | CAAGCAGAAGACGGCATACGAGAT GTTGGCTGGAGGCGAATTTCTCCGCTGTCAGGATCGA GTGACTGGAGTTCAGACGTGTGCTCTTCCGATC*T |
| 54 | 58 | CAAGCAGAAGACGGCATACGAGAT TCTTGGAGTTCATCACATTGCAATACCACTCCATGGT GTGACTGGAGTTCAGACGTGTGCTCTTCCGATC*T |
| 55 | 59 | CAAGCAGAAGACGGCATACGAGAT GACTAACGAGCGATCACTACCATGGCGACTTGAACGC GTGACTGGAGTTCAGACGTGTGCTCTTCCGATC*T |
| 56 | 60 | CAAGCAGAAGACGGCATACGAGAT CGTCTGTCCAGTTGACCTGGTCTGGTATTTACCATGC GTGACTGGAGTTCAGACGTGTGCTCTTCCGATC*T |
| 57 | 61 | CAAGCAGAAGACGGCATACGAGAT CCTGTGACTACTATCCATAACTCAACGCAAGTACGGT GTGACTGGAGTTCAGACGTGTGCTCTTCCGATC*T |
| 58 | 62 | CAAGCAGAAGACGGCATACGAGAT AGACCAATGGCACGAGACAGAATTGGCGGGTTACCTC GTGACTGGAGTTCAGACGTGTGCTCTTCCGATC*T |
| 59 | 63 | CAAGCAGAAGACGGCATACGAGAT GTGAAGTCTTGTCTTCTTTCGAAGGTATCTAGAGAGG GTGACTGGAGTTCAGACGTGTGCTCTTCCGATC*T |
| 60 | 64 | CAAGCAGAAGACGGCATACGAGAT GATGCAACTGGTAGAGCTTCCACTCCGTAAATCCGTG GTGACTGGAGTTCAGACGTGTGCTCTTCCGATC*T |
| 61 | 65 | CAAGCAGAAGACGGCATACGAGAT ATTGAGGCAACTGAACCTACGACATTCACGGATGACT GTGACTGGAGTTCAGACGTGTGCTCTTCCGATC*T |
| 62 | 66 | CAAGCAGAAGACGGCATACGAGAT TGGTGTCCTTAAGTGAGCAAGACCTAGATTGTGGTTC GTGACTGGAGTTCAGACGTGTGCTCTTCCGATC*T |
| 63 | 67 | CAAGCAGAAGACGGCATACGAGAT CAGTTGATATGCACACATTGGTGACTAAGAGATGCGT GTGACTGGAGTTCAGACGTGTGCTCTTCCGATC*T |
| 64 | 68 | CAAGCAGAAGACGGCATACGAGAT CAGACATTGGTACTGGCTTGGCTAGTCGACCAAGACA GTGACTGGAGTTCAGACGTGTGCTCTTCCGATC*T |
| 65 | 69 | CAAGCAGAAGACGGCATACGAGAT GCTTAATGGCGGATGAATTGCAGTCGTGGGGTACCAT GTGACTGGAGTTCAGACGTGTGCTCTTCCGATC*T |
| 66 | 70 | CAAGCAGAAGACGGCATACGAGAT CCTAGGATCATTGCCGCTCACCGCCAGAATTGGACAG GTGACTGGAGTTCAGACGTGTGCTCTTCCGATC*T |
| 67 | 71 | CAAGCAGAAGACGGCATACGAGAT GACGGTGCTCCTACTTATTGTCACCAGCCAACTCGAG GTGACTGGAGTTCAGACGTGTGCTCTTCCGATC*T |
| 68 | 72 | CAAGCAGAAGACGGCATACGAGAT CAGTGCCCGTCTTATACCCGTGTATTATGAGCGAGAA GTGACTGGAGTTCAGACGTGTGCTCTTCCGATC*T |
| 69 | 73 | CAAGCAGAAGACGGCATACGAGAT AATCGCTATCGGAGAGGTTCGCAGAAGCATCGGTTAG GTGACTGGAGTTCAGACGTGTGCTCTTCCGATC*T |
| 70 | 74 | CAAGCAGAAGACGGCATACGAGAT TCAGTCGAGGCCAATGCTTCTTCCTAGAGTTGTCCGT GTGACTGGAGTTCAGACGTGTGCTCTTCCGATC*T |
| 71 | 75 | CAAGCAGAAGACGGCATACGAGAT ACGTGAAGGTCAACCTCTTACCTACTGGCACTGTACG GTGACTGGAGTTCAGACGTGTGCTCTTCCGATC*T |
| 72 | 76 | CAAGCAGAAGACGGCATACGAGAT AAGCCATGAGGTGCTCATTATCGGCCTCCCGTGATCA GTGACTGGAGTTCAGACGTGTGCTCTTCCGATC*T |
| 73 | 77 | CAAGCAGAAGACGGCATACGAGAT GTGTTAGTGACTTCGCTCCAACAAGCTACCAGTCAAG GTGACTGGAGTTCAGACGTGTGCTCTTCCGATC*T |
| 74 | 78 | CAAGCAGAAGACGGCATACGAGAT GACTGCCCGGATTCATCGGCACTTAAGAACTAACACC GTGACTGGAGTTCAGACGTGTGCTCTTCCGATC*T |
| 75 | 79 | CAAGCAGAAGACGGCATACGAGAT CTGCATCTGGAGAATACTTGGCAGCATATTAGCGGTT GTGACTGGAGTTCAGACGTGTGCTCTTCCGATC*T |
| 76 | 80 | CAAGCAGAAGACGGCATACGAGAT TAACACCACCGCTTGTCTTCAATGGCAGTGGTCTTGT GTGACTGGAGTTCAGACGTGTGCTCTTCCGATC*T |
| 77 | 81 | CAAGCAGAAGACGGCATACGAGAT TGGCATTGTAAGCTGTCTTAAGGACGTTCATTCGGAC GTGACTGGAGTTCAGACGTGTGCTCTTCCGATC*T |
| 78 | 82 | CAAGCAGAAGACGGCATACGAGAT CGGTGTGTCGCGCAATGTTCTCGTTCCTTGGAACTGT GTGACTGGAGTTCAGACGTGTGCTCTTCCGATC*T |
| 79 | 83 | CAAGCAGAAGACGGCATACGAGAT TGTAGGAACGTAGCATATTCCGATTGTTGCTGTCTCT GTGACTGGAGTTCAGACGTGTGCTCTTCCGATC*T |
| 80 | 84 | CAAGCAGAAGACGGCATACGAGAT TCACTGACAGTGCAGCTTGAGGCCGAAGTTTATCGGC GTGACTGGAGTTCAGACGTGTGCTCTTCCGATC*T |
| 81 | 85 | CAAGCAGAAGACGGCATACGAGAT ACACTGGAGGTACGTATACGTATCCGAGGTTGCTCCA GTGACTGGAGTTCAGACGTGTGCTCTTCCGATC*T |
| 82 | 86 | CAAGCAGAAGACGGCATACGAGAT CTCTCTAGTGGCCTACACCAGCTTCTAGTTTCCACTG GTGACTGGAGTTCAGACGTGTGCTCTTCCGATC*T |
| 83 | 87 | CAAGCAGAAGACGGCATACGAGAT GACTTATACGTGCGAAGTTCAAGATTGCCCAGGCATT GTGACTGGAGTTCAGACGTGTGCTCTTCCGATC*T |
| 84 | 88 | CAAGCAGAAGACGGCATACGAGAT TCACGCCGTTCTATGGCCCGGTTACAATTACGACACT GTGACTGGAGTTCAGACGTGTGCTCTTCCGATC*T |
| 85 | 89 | CAAGCAGAAGACGGCATACGAGAT AGGTGGAATGACAGCGCTTCATAACACCGTCAAGTGG GTGACTGGAGTTCAGACGTGTGCTCTTCCGATC*T |
| 86 | 90 | CAAGCAGAAGACGGCATACGAGAT TTGAGCATGAACTGCATTCAGTGAAGTCGTTGGCACT GTGACTGGAGTTCAGACGTGTGCTCTTCCGATC*T |
| 87 | 91 | CAAGCAGAAGACGGCATACGAGAT CAATCAGCCGTATGGTCGGAGACACTCCTTATGCACC GTGACTGGAGTTCAGACGTGTGCTCTTCCGATC*T |
| 88 | 92 | CAAGCAGAAGACGGCATACGAGAT TATCAACATGCTTGCTTTACGGCCAACTTCCGATAAC GTGACTGGAGTTCAGACGTGTGCTCTTCCGATC*T |
| 89 | 93 | CAAGCAGAAGACGGCATACGAGAT TATGGAGCGTTGCTTAATCAGAGCAACTTCCGATAAC GTGACTGGAGTTCAGACGTGTGCTCTTCCGATC*T |
| 90 | 94 | CAAGCAGAAGACGGCATACGAGAT TGCATTAGTTCGCACTCTGCCTATACTCGGAAGTTCC GTGACTGGAGTTCAGACGTGTGCTCTTCCGATC*T |
| 91 | 95 | CAAGCAGAAGACGGCATACGAGAT TGAATGGATAGGAGCCATTGCCTTCCGATGCTTCAGA GTGACTGGAGTTCAGACGTGTGCTCTTCCGATC*T |
| 92 | 96 | CAAGCAGAAGACGGCATACGAGAT ATCTATGCTATGGCAGGTTCTTAACCGACGTGACAGA GTGACTGGAGTTCAGACGTGTGCTCTTCCGATC*T |
| 93 | 97 | CAAGCAGAAGACGGCATACGAGAT CGGCTATGGTTAGAACGTAACGTGGTAGGTACCGTCA GTGACTGGAGTTCAGACGTGTGCTCTTCCGATC*T |
| 94 | 98 | CAAGCAGAAGACGGCATACGAGAT TTAGCGGACAATCTCCTTTAACGCATGGTTACAAGCC GTGACTGGAGTTCAGACGTGTGCTCTTCCGATC*T |
| 95 | 99 | CAAGCAGAAGACGGCATACGAGAT CCACAATTCTCTAAGACGGTCGCGTACAAGATCGAAG GTGACTGGAGTTCAGACGTGTGCTCTTCCGATC*T |
| 96 | 100 | CAAGCAGAAGACGGCATACGAGAT CAACCACGACTACTGCGTTGAACTGAGGAGGTTCGTT GTGACTGGAGTTCAGACGTGTGCTCTTCCGATC*T |

| | SEQ ID |
|---|---|
| i5/P5 primer | NO |
| AATGATACGGCGACCACCGAGATCTACAC ACAAGCGTCCACTATAGGGAGCAAGAAGCTTGCAGTC ACACTCTTTCCCTACACGACGCTCTTCCGATC*T | 101 |
| AATGATACGGCGACCACCGAGATCTACAC TACAGTCGCACCGAACATGTATCACACGTGTGTCTAC ACACTCTTTCCCTACACGACGCTCTTCCGATC*T | 102 |
| AATGATACGGCGACCACCGAGATCTACAC GAGTATCCACATATAGCTTCTGACGGCGAGTATGGAC ACACTCTTTCCCTACACGACGCTCTTCCGATC*T | 103 |
| AATGATACGGCGACCACCGAGATCTACAC CTTCAAGATGTCTGCCGTCGCGGAGTTACAGAGTGGA ACACTCTTTCCCTACACGACGCTCTTCCGATC*T | 104 |
| AATGATACGGCGACCACCGAGATCTACAC ATCAATACTTGTGGCTGTCAACCACTTGACTTACTGG ACACTCTTTCCCTACACGACGCTCTTCCGATC*T | 105 |
| AATGATACGGCGACCACCGAGATCTACAC AGTCTTGCAAGTACGCCTGTGTATTGGACTCATGAGC ACACTCTTTCCCTACACGACGCTCTTCCGATC*T | 106 |
| AATGATACGGCGACCACCGAGATCTACAC TGCCTATAGACCACCGATTGCGCCTGACTGCCATCTT ACACTCTTTCCCTACACGACGCTCTTCCGATC*T | 107 |
| AATGATACGGCGACCACCGAGATCTACAC AGAGATGCGGCGTACTATTCTGCCACGCTTAGCTTGG ACACTCTTTCCCTACACGACGCTCTTCCGATC*T | 108 |
| AATGATACGGCGACCACCGAGATCTACAC ATTGCATTATTACGAGGTTGATTAGGAGGAGTGCATC ACACTCTTTCCCTACACGACGCTCTTCCGATC*T | 109 |
| AATGATACGGCGACCACCGAGATCTACAC GTAGTAGAGTACAGACACCACAGGCATTATCGTTCGA ACACTCTTTCCCTACACGACGCTCTTCCGATC*T | 110 |
| AATGATACGGCGACCACCGAGATCTACAC CGCAATTTCCGCATTATAACCACGGAGGTGGAACAAC ACACTCTTTCCCTACACGACGCTCTTCCGATC*T | 111 |
| AATGATACGGCGACCACCGAGATCTACAC CGAGCTGCATCAGGCGTTCATGCATGAACACCTCGTT ACACTCTTTCCCTACACGACGCTCTTCCGATC*T | 112 |
| AATGATACGGCGACCACCGAGATCTACAC GAACGGTTCAACTCGCGTTACGGTGCTATTTGCGTGT ACACTCTTTCCCTACACGACGCTCTTCCGATC*T | 113 |
| AATGATACGGCGACCACCGAGATCTACAC TGTCTCCGCAACAACGCTTCTCCATTACGAGTTGCCA ACACTCTTTCCCTACACGACGCTCTTCCGATC*T | 114 |
| AATGATACGGCGACCACCGAGATCTACAC TTCGCCACGTTACGGTATTCGAGTCGGACGTGGTCAA ACACTCTTTCCCTACACGACGCTCTTCCGATC*T | 115 |
| AATGATACGGCGACCACCGAGATCTACAC TGTGCGACTCGCTAATCTTAGCCTGGTCCTTCAGACG ACACTCTTTCCCTACACGACGCTCTTCCGATC*T | 116 |
| AATGATACGGCGACCACCGAGATCTACAC AAGACGTGGACTAACGTTTACTGTATGCGGCTACTAG ACACTCTTTCCCTACACGACGCTCTTCCGATC*T | 117 |
| AATGATACGGCGACCACCGAGATCTACAC ATGGAGTCGGTAATAGTCCTCTAGAATGAATTCGTGG ACACTCTTTCCCTACACGACGCTCTTCCGATC*T | 118 |
| AATGATACGGCGACCACCGAGATCTACAC AACGAGGTCGACGTTGTGACGAGCAGTTGCCTGATTC ACACTCTTTCCCTACACGACGCTCTTCCGATC*T | 119 |
| AATGATACGGCGACCACCGAGATCTACAC AGCTTGCCGGACTTAGGTTCCTGGCTTCTAATGCCAG ACACTCTTTCCCTACACGACGCTCTTCCGATC*T | 120 |
| AATGATACGGCGACCACCGAGATCTACAC CCGATCGGTTAGTAGCTTTCGCATACTGTTCATCTCC ACACTCTTTCCCTACACGACGCTCTTCCGATC*T | 121 |
| AATGATACGGCGACCACCGAGATCTACAC TGCATCCTCCTCAGGAGTTCATGTCCATCGAACCGTT ACACTCTTTCCCTACACGACGCTCTTCCGATC*T | 122 |
| AATGATACGGCGACCACCGAGATCTACAC TTCTAGCTGGCCATAACTGAACAGGCACCCAGAATGC ACACTCTTTCCCTACACGACGCTCTTCCGATC*T | 123 |
| AATGATACGGCGACCACCGAGATCTACAC CATGATGCTTCAACGTGTGAGCGAATACATGTGACCT ACACTCTTTCCCTACACGACGCTCTTCCGATC*T | 124 |
| AATGATACGGCGACCACCGAGATCTACAC TTAGTCAGCGTTAGATTTCCACGGTTCCAGCATTGCT ACACTCTTTCCCTACACGACGCTCTTCCGATC*T | 125 |
| AATGATACGGCGACCACCGAGATCTACAC CAGTTGCGATTAGGTCCTCAGACTCCTCGCTTCTTGC ACACTCTTTCCCTACACGACGCTCTTCCGATC*T | 126 |
| AATGATACGGCGACCACCGAGATCTACAC ATCGGTTGTCCATGTTATGCGCTAAGTATGAGACATG ACACTCTTTCCCTACACGACGCTCTTCCGATC*T | 127 |
| AATGATACGGCGACCACCGAGATCTACAC AGTCTCGTCCGGCCAATTGGAGTTCTTCCCTGGTACA ACACTCTTTCCCTACACGACGCTCTTCCGATC*T | 128 |
| AATGATACGGCGACCACCGAGATCTACAC CAATCGAACAGTCACGCTTCTCTTCAGGATCTCGGAT ACACTCTTTCCCTACACGACGCTCTTCCGATC*T | 129 |
| AATGATACGGCGACCACCGAGATCTACAC ACGTGGTGACCGTAGACGAGGAATTCCGCACTTGAGG ACACTCTTTCCCTACACGACGCTCTTCCGATC*T | 130 |
| AATGATACGGCGACCACCGAGATCTACAC AGAGTCTAGTCCGGTGTTTCCTTGTGTACCTTGTGGT ACACTCTTTCCCTACACGACGCTCTTCCGATC*T | 131 |
| AATGATACGGCGACCACCGAGATCTACAC GTGATGGGCGCACTAGTTGCCAGCTAACACAGTAACC ACACTCTTTCCCTACACGACGCTCTTCCGATC*T | 132 |
| AATGATACGGCGACCACCGAGATCTACAC GTCCAGGTGGCTGAAGCTTCACCTAGGTTTCGTGCTT ACACTCTTTCCCTACACGACGCTCTTCCGATC*T | 133 |
| AATGATACGGCGACCACCGAGATCTACAC TCTCAGCGACACAGTGCGCTTGATCATTCAACGAGCT ACACTCTTTCCCTACACGACGCTCTTCCGATC*T | 134 |
| AATGATACGGCGACCACCGAGATCTACAC CGTAGGTGTATTGCCTATTGTTGAGATCGACTCCTGA ACACTCTTTCCCTACACGACGCTCTTCCGATC*T | 135 |
| AATGATACGGCGACCACCGAGATCTACAC TTCAGGTCCACGGTAACTAGCTGAAGAGTTCTGCCAT ACACTCTTTCCCTACACGACGCTCTTCCGATC*T | 136 |
| AATGATACGGCGACCACCGAGATCTACAC GAACCGACGTATCGCGATTCGACCACAACTCTCCAAG ACACTCTTTCCCTACACGACGCTCTTCCGATC*T | 137 |
| AATGATACGGCGACCACCGAGATCTACAC ACCTGCGCGACATCGACTCCGAAGAATAGTTGCCAAC ACACTCTTTCCCTACACGACGCTCTTCCGATC*T | 138 |
| AATGATACGGCGACCACCGAGATCTACAC CTCATAGTCCATATGCCCTCGTAATTGTCATCTGCAG ACACTCTTTCCCTACACGACGCTCTTCCGATC*T | 139 |
| AATGATACGGCGACCACCGAGATCTACAC AGGCCTGCGCTATTAAGGGCGTCCATTAACCTAGCTA ACACTCTTTCCCTACACGACGCTCTTCCGATC*T | 140 |
| AATGATACGGCGACCACCGAGATCTACAC ACAGAACTGCATTAGACTTCACTCTTAGCCGCAATTC ACACTCTTTCCCTACACGACGCTCTTCCGATC*T | 141 |
| AATGATACGGCGACCACCGAGATCTACAC CGTTAGGAGCATGTTGTTTGCCGGAAGACCGCTCTAA ACACTCTTTCCCTACACGACGCTCTTCCGATC*T | 142 |
| AATGATACGGCGACCACCGAGATCTACAC GAACACCTAATCTGGCTTTAATCGGTGAGGGCTTCTA ACACTCTTTCCCTACACGACGCTCTTCCGATC*T | 143 |
| AATGATACGGCGACCACCGAGATCTACAC CTTGCACACAGGATGGTTTCCGCAACCAACCTCAAGA ACACTCTTTCCCTACACGACGCTCTTCCGATC*T | 144 |
| AATGATACGGCGACCACCGAGATCTACAC GATCTTGGCTCTGTAGGTTCGGTTAGCTCTGGACGTT ACACTCTTTCCCTACACGACGCTCTTCCGATC*T | 145 |
| AATGATACGGCGACCACCGAGATCTACAC AGCACTTAGTTCTCAGGTTCGGAACTTCAACACGAAG ACACTCTTTCCCTACACGACGCTCTTCCGATC*T | 146 |
| AATGATACGGCGACCACCGAGATCTACAC GCCACTCTCAGCATAGCTCCAAGTCTCTTACGTACTC ACACTCTTTCCCTACACGACGCTCTTCCGATC*T | 147 |
| AATGATACGGCGACCACCGAGATCTACAC GGTATTGATAGAGAAGCTTAGTGGCGCCACCACAGAT ACACTCTTTCCCTACACGACGCTCTTCCGATC*T | 148 |
| AATGATACGGCGACCACCGAGATCTACAC GTATGCGGACCTCACCTTTCCACCGAAGGCCTAGAGT ACACTCTTTCCCTACACGACGCTCTTCCGATC*T | 149 |
| AATGATACGGCGACCACCGAGATCTACAC ACGATGATCGTCGATTATTGAGGCTCTACTCCTTGAC ACACTCTTTCCCTACACGACGCTCTTCCGATC*T | 150 |
| AATGATACGGCGACCACCGAGATCTACAC GTTGAACCTAGTACAAGTTAGGACACAGAGCAAGTCT ACACTCTTTCCCTACACGACGCTCTTCCGATC*T | 151 |
| AATGATACGGCGACCACCGAGATCTACAC AGCAGTCTCAACCGGTATGCGGATTCATATAAGCGTC ACACTCTTTCCCTACACGACGCTCTTCCGATC*T | 152 |
| AATGATACGGCGACCACCGAGATCTACAC GAAGCGGCGAACCAGCACCGGATGTAAGGAACATCCG ACACTCTTTCCCTACACGACGCTCTTCCGATC*T | 153 |
| AATGATACGGCGACCACCGAGATCTACAC TCGTCACCTCCATAGAATACGACAAGGCGCTCCAGTT ACACTCTTTCCCTACACGACGCTCTTCCGATC*T | 154 |
| AATGATACGGCGACCACCGAGATCTACAC ATCATCTCGCTGTACGTTTCTAGAGGAAGGTGTGAAG ACACTCTTTCCCTACACGACGCTCTTCCGATC*T | 155 |
| AATGATACGGCGACCACCGAGATCTACAC CCACTCCGTATCAAGCAGGCGATTGAGGTTCCGAACT ACACTCTTTCCCTACACGACGCTCTTCCGATC*T | 156 |
| AATGATACGGCGACCACCGAGATCTACAC GCAAGTTAGCACACGAGTTCGTACGCTAAACCGGATT ACACTCTTTCCCTACACGACGCTCTTCCGATC*T | 157 |
| AATGATACGGCGACCACCGAGATCTACAC TTACTTCATGACGAACATTCTTGTAACGGTATGGCCA ACACTCTTTCCCTACACGACGCTCTTCCGATC*T | 158 |
| AATGATACGGCGACCACCGAGATCTACAC GTTAATGATGCTCGGCATGTAAGGTGGCTGAGAACCA ACACTCTTTCCCTACACGACGCTCTTCCGATC*T | 159 |
| AATGATACGGCGACCACCGAGATCTACAC CTAACGGTCACTACGGAAACCGCTCCAACTCACACAC ACACTCTTTCCCTACACGACGCTCTTCCGATC*T | 160 |
| AATGATACGGCGACCACCGAGATCTACAC TACTTGGGCGATAGGAGTGCGGTGGATTCACCAAGGA ACACTCTTTCCCTACACGACGCTCTTCCGATC*T | 161 |
| AATGATACGGCGACCACCGAGATCTACAC GTTCACTGACATGACTAGTACAACTCGAGTTCACGCT ACACTCTTTCCCTACACGACGCTCTTCCGATC*T | 162 |
| AATGATACGGCGACCACCGAGATCTACAC TGAGAGTCGACATATCTTTGACACGGTGCACTCTGCT ACACTCTTTCCCTACACGACGCTCTTCCGATC*T | 163 |
| AATGATACGGCGACCACCGAGATCTACAC TCTTCGGGTCTAGTCTTTTCACGCGTCTGCTACCTAC ACACTCTTTCCCTACACGACGCTCTTCCGATC*T | 164 |
| AATGATACGGCGACCACCGAGATCTACAC CTAGAGATGTCCTCATATTGAATCCGGTTGGTATTCC ACACTCTTTCCCTACACGACGCTCTTCCGATC*T | 165 |
| AATGATACGGCGACCACCGAGATCTACAC CATTCGGGCTGGCTGATACAGGAAGGAGAGAATCCTC ACACTCTTTCCCTACACGACGCTCTTCCGATC*T | 166 |
| AATGATACGGCGACCACCGAGATCTACAC CTGTATTCTGTAATCTCGCAATCAGATCATTCGGTCT ACACTCTTTCCCTACACGACGCTCTTCCGATC*T | 167 |
| AATGATACGGCGACCACCGAGATCTACAC TCTACCACATTAACGGCTGAGGTACTTGTAGTGAAGG ACACTCTTTCCCTACACGACGCTCTTCCGATC*T | 168 |
| AATGATACGGCGACCACCGAGATCTACAC TCTCGTCCGCCAATACCTTACGTCGAGTAGATCTTCG ACACTCTTTCCCTACACGACGCTCTTCCGATC*T | 169 |
| AATGATACGGCGACCACCGAGATCTACAC CTTACCTACGAACTTCATTCTTCTTCTCCCAGGTTGA ACACTCTTTCCCTACACGACGCTCTTCCGATC*T | 170 |
| AATGATACGGCGACCACCGAGATCTACAC CCACGGTGTCGAATCGCTGACAACCTCTCCTACCAGA ACACTCTTTCCCTACACGACGCTCTTCCGATC*T | 171 |
| AATGATACGGCGACCACCGAGATCTACAC TCTACCGGCAAGACTCTTTCTCTCGAACTATCCTGTC ACACTCTTTCCCTACACGACGCTCTTCCGATC*T | 172 |
| AATGATACGGCGACCACCGAGATCTACAC TGTGCACGGCATATCCGTTCTCTGGCCAATAACTGCC ACACTCTTTCCCTACACGACGCTCTTCCGATC*T | 173 |
| AATGATACGGCGACCACCGAGATCTACAC TCGTCGTTCTCACACGGTTCCTTAAGCCTACCGTTGA ACACTCTTTCCCTACACGACGCTCTTCCGATC*T | 174 |
| AATGATACGGCGACCACCGAGATCTACAC AGCTCTAGGCTTACACTTGCAACGTATTGCATCTGAC ACACTCTTTCCCTACACGACGCTCTTCCGATC*T | 175 |
| AATGATACGGCGACCACCGAGATCTACAC TCGTAATTCCGGTACCTTGACACAACAGGACGCATAG ACACTCTTTCCCTACACGACGCTCTTCCGATC*T | 176 |
| AATGATACGGCGACCACCGAGATCTACAC CCGTCTTCCACACAAGATTCTACAGACTCTGGTTGTG ACACTCTTTCCCTACACGACGCTCTTCCGATC*T | 177 |
| AATGATACGGCGACCACCGAGATCTACAC ATGAGATGTTATGACGCTGCTACCTGTAATCGAATCG ACACTCTTTCCCTACACGACGCTCTTCCGATC*T | 178 |
| AATGATACGGCGACCACCGAGATCTACAC TAGGACCGGAGTTGCTTCCATATTCGGAATTGAGCTG ACACTCTTTCCCTACACGACGCTCTTCCGATC*T | 179 |
| AATGATACGGCGACCACCGAGATCTACAC TCTTATTCATACGTTCATTCCGCCAATTCCAAGGTAC ACACTCTTTCCCTACACGACGCTCTTCCGATC*T | 180 |
| AATGATACGGCGACCACCGAGATCTACAC GGCTTCTGGTTGGTTCTTGGATAACGATCCAACCTTG ACACTCTTTCCCTACACGACGCTCTTCCGATC*T | 181 |
| AATGATACGGCGACCACCGAGATCTACAC GAGCGGTCCACTGACAAGTCGGATAACCGATACGCTC ACACTCTTTCCCTACACGACGCTCTTCCGATC*T | 182 |
| AATGATACGGCGACCACCGAGATCTACAC GCCAGAACGATTGTCCGTACTAGGCGGTGGGCTGTAT ACACTCTTTCCCTACACGACGCTCTTCCGATC*T | 183 |
| AATGATACGGCGACCACCGAGATCTACAC TAGACCAAGCCTAACCATTGCGTTATAGCATAAGGCG ACACTCTTTCCCTACACGACGCTCTTCCGATC*T | 184 |
| AATGATACGGCGACCACCGAGATCTACAC GAGGAGATGCTCACATCTTCTCACCTCATTTGCAAGG ACACTCTTTCCCTACACGACGCTCTTCCGATC*T | 185 |
| AATGATACGGCGACCACCGAGATCTACAC ACACCTCCTCTGCGAGATGGCATATTACCGATTGGTC ACACTCTTTCCCTACACGACGCTCTTCCGATC*T | 186 |
| AATGATACGGCGACCACCGAGATCTACAC TGGCAATCAGTCGCTGAGGAAGGACTCTGTTGCCTTG ACACTCTTTCCCTACACGACGCTCTTCCGATC*T | 187 |
| AATGATACGGCGACCACCGAGATCTACAC TGTTGATTCAAGTGTCATAGGATGATGGCGGAAGAGA ACACTCTTTCCCTACACGACGCTCTTCCGATC*T | 188 |
| AATGATACGGCGACCACCGAGATCTACAC GCTGACACCTGATAGCCTTACACGTAACCCAATCGGA ACACTCTTTCCCTACACGACGCTCTTCCGATC*T | 189 |
| AATGATACGGCGACCACCGAGATCTACAC GAGGTTCTGTGTTCGTCGGAGATCCAACATGACATCC ACACTCTTTCCCTACACGACGCTCTTCCGATC*T | 190 |
| AATGATACGGCGACCACCGAGATCTACAC ACCTCAGGAAGTTACAGTCAAGAACCGTGCAGTGGAT ACACTCTTTCCCTACACGACGCTCTTCCGATC*T | 191 |
| AATGATACGGCGACCACCGAGATCTACAC ACCTGACCACGACACCATTGGCTTCAACGGGATAGGT ACACTCTTTCCCTACACGACGCTCTTCCGATC*T | 192 |
| AATGATACGGCGACCACCGAGATCTACAC TAAGATCACTGCGTCCATGGAGTGTGCATCTAGTGAG ACACTCTTTCCCTACACGACGCTCTTCCGATC*T | 193 |
| AATGATACGGCGACCACCGAGATCTACAC CAAGAGGGGTGCCTATCTAGCAGCTCTTATTAGGTGC ACACTCTTTCCCTACACGACGCTCTTCCGATC*T | 194 |
| AATGATACGGCGACCACCGAGATCTACAC ATCGAGAGGTGAATTCATTGGAGGTTAGATGGCATGA ACACTCTTTCCCTACACGACGCTCTTCCGATC*T | 195 |
| AATGATACGGCGACCACCGAGATCTACAC GACACGTTATCAGCTGGTTATGAGTCACGTGCTTAGC ACACTCTTTCCCTACACGACGCTCTTCCGATC*T | 196 |

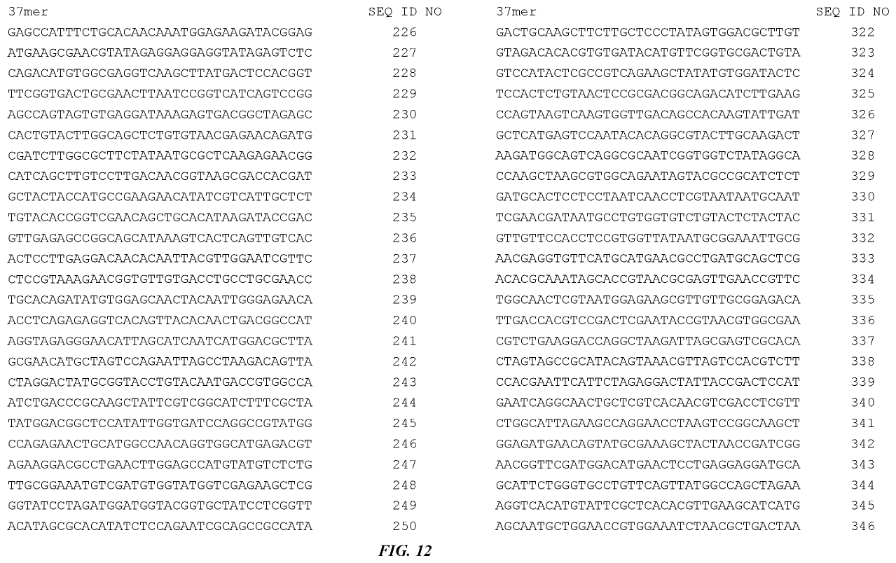

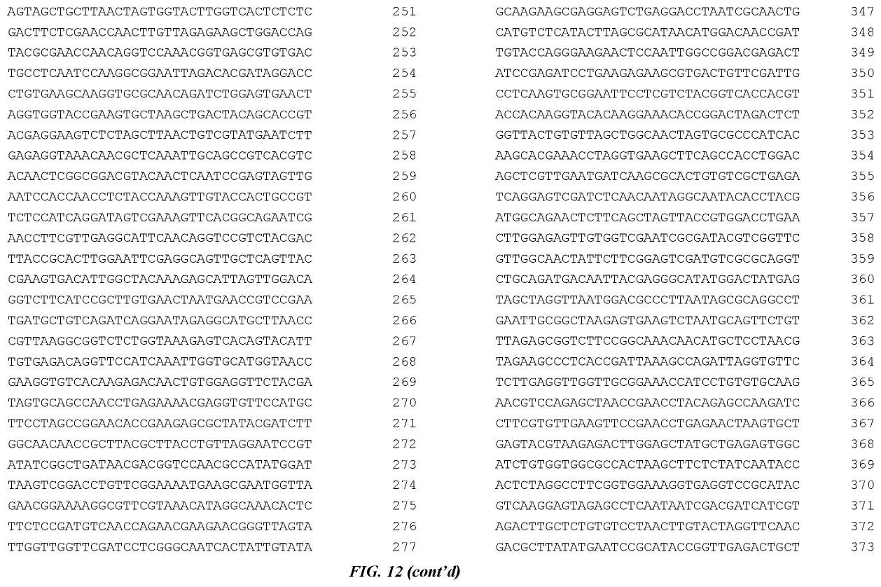

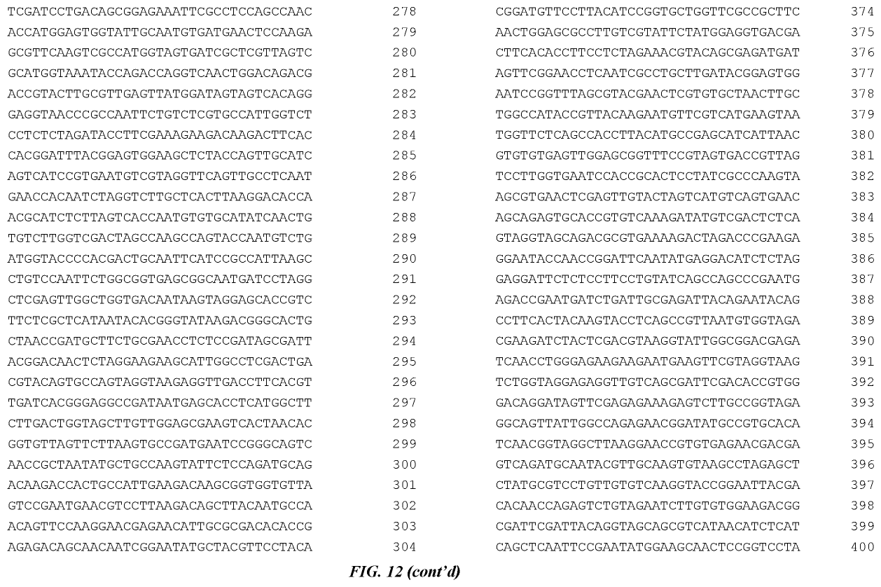

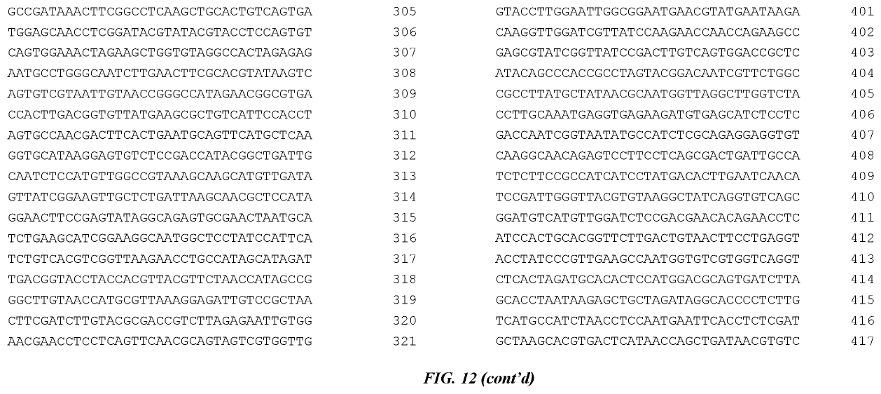

Dual-use protocol for Illumina and nanopore sequencing platforms. Multiplexing barcodes on the Illumina platform typically have the lengths of 8 nt flanking the sequence read on both ends, but they are not ideal for multiplexing samples being sequenced on a nanopore instrument (Oxford Nanopore Technologies) due to the higher error rate of this platform. A dual-use barcode system was designed that contains, in an exemplary embodiment, a distinct 37 nucleotide (nt) barcode on each side of sequencing adaptor (the first 8 nt of which were used for Illumina multiplexing), which enables the multiplexed DNA library to be sequenced on both Illumina and nanopore platforms. The barcodes were designed using an in-house developed R script to generate 8 nt and 29 nt barcodes that maximized the Levenshtein distance between any given pair of barcodes. Specifically, the DNA Barcodes package in BioConductor was first used to generate a set of 1,014 unique 10mers with minimum Levenshtein distance of 4 and a set of 283 unique 8mers with minimum Levenshtein distance of 3, as computational limitations prevented design of 37mers directly. An 8mer and three 10mers were then concatenated together with stripping of the last nt to generate a 37mer index primer. The final set of 192 37mer (FIG. 12) index primers had a mean Levenshtein distance of 23+/−2.4 SD (range 14-31) between any two barcodes.

Pipeline for Designing 37mer Barcodes for Dual Use of Illumina and Oxford Nanopore Technologies (Nanopore) Sequencing Instruments. 37mer barcodes that could effectively be used for both Illumina and Nanopore were designed with the following goals in mind: generate 192 37mer barcodes such that unique dual indexes (UDIs) can be used for all 96 samples on a 96-well plate; maximizing Levenshtein distance between each pair of barcodes in order to minimize barcode “crosstalk”; Ensuring a minimum Levenshtein distance between each pair of 8mers (the first 8 nt (nucleotides) of each 37mer) for Illumina sequencing; and ensuring a minimum Levenshtein distance between each pair of 37mers for nanopore sequencing on Oxford Nanopore Technologies (ONT) instruments. It was determined that it was too computationally expensive to determine DNA barcodes that maximize the Levenshtein distance between them for barcodes of >12 nt in length. Instead, the strategy was to design a set of 8mer barcodes and 3 sets of unique 10mer barcodes that are concatenated together to form 37mer barcodes.

A custom algorithm was developed in python to carry out the above strategy. For the python code, the following scripts were utilized: Run the R script “generate_8mer barcodes.R”. This script uses the DNABarcodes tool in the Bioconductor software package (v.3.1.1). These scripts were run in R v4.0. Required additional R packages include Matrix and parallel. Note that setting the Levenshtein distance threshold to 3 will not yield a sufficient number of candidate barcodes (n=187). Thus, barcodes generated from setting the Levenshtein distance threshold to 3 and then rerunning the R script with the Hamming distance threshold set to 3 were combined.

This R script generates a total of 914 candidate barcodes at a Levenshtein or Hamming distance threshold of 3. Note that the DNABarcodes algorithm is non-coordinated, meaning that it will not generate identical results when the program is rerun. The pairwise distances between any two barcodes can be calculated using Linux commands that will also auto-generate an R script.

The in-house Python script “parse_lev_distance.py” can then be used to pull out all barcodes at a predefined minimum Levenshtein distance threshold of 3.

It was found that there were 283 8mer barcodes at a Levenshtein threshold cutoff of 3 generated in total (“8mer_barcodes.txt”). The minimum Levenshtein distance for any given set of barcodes can be separately checked with a Bash shell script “check_distance_lev.sh”.

Next, to generate the remaining 29 nt stretch of the 37mer barcodes, the DNABarcodes tool was used to generate 12mer (“generate_12mer_barcodes.R”) and 10mer (“generate_10mer_barcodes.R”) barcodes and then concatenate 1 12mer and 2 10mer barcodes to generate a 32mer barcode, from which 3 nucleotides can be stripped to generate 29mer barcodes.

This R script generated 232 12mer barcodes and 1,014 10 mer barcodes. The pairwise distances between any two barcodes can be calculated using Linux commands, and which also auto-generate an R script.

An in-house Python script “parse_lev_distance.py” can then be used to pull out all barcodes at a predefined minimum Levenshtein distance threshold of 4.

Use of the script results in 232 12mer barcodes (“12mer_barcodes.txt”) and 1,014 10mer barcodes (“10mer_barcodes.txt”). Note the minimum Levenshtein distances cannot be increased by 1 to 6 and 5, because this will decrease the number of usable barcodes to less than 192 (45 and 72, respectively).

Next, randomly from the pool of 232 12mer barcodes (“12mer_barcodes.txt”) and 1,014 10mer barcodes (“10mer_barcodes.txt”), barcodes were selected and concatenated these chosen barcodes to generate 232 32mer barcodes (12mer-10 mer-10 mer) (“32mer_barcodes.txt”). 3 nucleotides were stripped off of the 3′ end and then concatenated to a random 8mer barcode from the pool of 238 8mer barcodes (“8mer_barcodes.txt”) to the 5′ end to generate 37mer barcodes. Finally, 192 of the “best” 37mer barcodes (“37mer_barcodes.txt”) were selected on the basis of maximum pairwise Levenshtein distance selected using similar Linux commands as described above. The minimum pairwise Levenshtein distance for this set of 192 37mer barcodes is 14 using the “check_distance_lev.sh” shell script.

Illumina sequencing. DNA libraries were pooled in equal volumes and the sequencing library pool was quantified using the Qubit fluorometer (ThermoFisher). Illumina sequencing was performed on MiSeq (2×150 nt paired-end)(with capacity for up to 5 samples per run) or HiSeq 1500/2500 instruments (140 nt single or 2×140 nt paired-end, with capacity for up to 40 samples per lane), according to the manufacturer's protocol.

Nanopore sequencing. Stringent procedures were adopted to prevent cross-contamination between samples during the library preparation steps, including unidirectional workflow, separating pre-PCR and post-PCR workspaces, and regular cleaning of the workbenches and biosafety cabinets with 5% sodium hypochlorite. Amplified DNA libraries were prepared for nanopore sequencing using the 1D library preparation kit (Oxford Nanopore Technologies) either manually or on an epMotion 5075 liquid handler biorobot (Eppendorf), with the processing of 8-16 samples per batch. The input DNA ranged from 200-1000 ng. The DNA was then sequenced using either R9.4 or R9.5 flow cells on a MinION or GridION X5 instrument (Oxford Nanopore Technologies). The MinION has a single flow cell position for processing of a single sample at a time, while the GridION has 5 flow cell positions for processing of up of 5 samples simultaneously. Up to five individually barcoded samples per flow cell were sequentially loaded on the nanopore instrument for sequencing. Between each sample, flow cells were washed according to the manufacturer's instructions to minimize carryover contamination. The estimated cost for reagents per sample (excluding labor) was $27.20-$61.40 and $269.70 for Illumina and nanopore sequencing, 589 respectively.

Positive and negative external controls. Negative controls were from the same batch of pooled plasma from healthy donors (Golden West Biologicals, CA). Positive controls consist of the negative control plasma spiked with sheared (to 150-200 base pair range) DNA extracted from cultured non-pathogenic microorganisms (American Type Culture Collection, VA): Koi herpesvirus (virus, VT-1592D), Streptococcus uberis (gram-positive bacterium, ATCC strain 0140J BAA-854D-5), Rhodobacter sphaeroides (gram-negative bacterium, ATCC BAA-808D-5), Millerozyma farinosa (yeast, ATCC MYA-4447D-5), Aspergillus oryzae (mold, ATCC 42149D-2), and Neospora caninum (parasite, ATCC 50843D) (see Table 3). All controls underwent the same wet lab procedure and bioinformatics analysis as the clinical samples.

TABLE 3
Positive and negative external controls.

| | | | | | | | | Titer of |
|---|---|---|---|---|---|---|---|---|
| | | | | | | | | spiked |
| | | | | | | | | organism |
| | | | | | | | | in + |
| | | | Organism | Reads | | | | control |
| Sample | Platform | Reads | detected | (>2) | RPM | Ct | nRPM | (cp/mL) |
| NC-1 | Illumina | 6,415,971 | No hits | n/a | n/a | 10.5 | n/a | n/a |
| NC-2 | Illumina | 12,520,990 | Thermus | 20 | 1.6 | 10.5 | 0.141 | n/a |
| | | | scotoductus | | | | | |
| | | | Propionibacterium | 14 | 1.1 | 10.5 | 0.099 | n/a |
| | | | acnes | | | | | |
| | | | Methylobacterium | 12 | 1.0 | 10.5 | 0.085 | n/a |
| | | | radiotolerans | | | | | |
| | | | Variovorax | 10 | 0.8 | 10.5 | 0.071 | n/a |
| | | | paradoxus | | | | | |
| NC-3 | Illumina | 7,545,060 | Propionibacterium | 18 | 2.4 | 10.5 | 0.211 | n/a |
| | | | acnes | | | | | |
| | | | Achromobacter | 14 | 1.9 | 10.5 | 0.164 | n/a |
| | | | xylosoxidans | | | | | |
| | | | Pseudomonas | 7 | 0.9 | 10.5 | 0.082 | n/a |
| | | | pseudoalcaligenes | | | | | |
| | | | Thermus | 4 | 0.5 | 10.5 | 0.047 | n/a |
| | | | scotoductus | | | | | |
| NC-4 | Illumina | 1,244,108 | Verminephrobacter | 3 | 2.4 | 10.5 | 0.213 | n/a |
| | | | eiseniae | | | | | |
| | | | Comamonas | 3 | 2.4 | 10.5 | 0.213 | n/a |
| | | | testosteroni | | | | | |
| NC-5 | Illumina | 11,598,931 | No hits | n/a | n/a | 10.5 | n/a | n/a |
| NC-6 | Illumina | 13,599,694 | No hits | n/a | n/a | 10.5 | n/a | n/a |
| NC-7 | Nanopore | 702,446 | Achromobacter | 26 | 37.0 | 10.5 | 3.272 | n/a |
| | | | xylosoxidans | | | | | |
| | | | Cutibacterium | 6 | 8.5 | 10.5 | 0.755 | n/a |
| | | | acnes | | | | | |
| | | | Pseudomonas | 5 | 7.1 | 10.5 | 0.629 | n/a |
| | | | fluorescens | | | | | |
| NC-8 | Nanopore | 1,188,000 | No hits | n/a | n/a | 10.5 | 0.000 | n/a |
| PC-1 | Illumina | 8,602,231 | Cyprinid | 9483 | 1102.4 | 12 | 34.450 | 6076 |
| | | | herpesvirus 3 | | | | | |
| | | | Streptococcus | 10084 | 1172.3 | 12 | 36.633 | 26430 |
| | | | uberis | | | | | |
| | | | Rhodobacter | 7827 | 909.9 | 12 | 28.434 | 159002 |
| | | | sphaeroides | | | | | |
| | | | Millerozyma | 15276 | 1775.8 | 12 | 55.494 | 27309 |
| | | | farinosa | | | | | |
| | | | Aspergillus oryzae | 4502 | 523.4 | 12 | 16.355 | 40 |
| | | | Neospora caninum | 2926 | 340.1 | 12 | 10.630 | 202 |
| | | | Thermus | 8 | 0.9 | 12 | 0.029 | |
| | | | scotoductus | | | | | |
| | | | Achromobacter | 7 | 0.8 | 12 | 0.025 | |
| | | | xylosoxidans | | | | | |
| | | | Propionibacterium | 6 | 0.7 | 12 | 0.022 | |
| | | | acnes | | | | | |
| | | | Pseudomonas | 7 | 0.8 | 12 | 0.025 | |
| | | | pseudoalcaligenes | | | | | |
| PC-2 | Nanopore | 3,119,414 | Cyprinid | 6,907 | 2,214 | 12 | 69.194 | 6076 |
| | | | herpesvirus 3 | | | | | |
| | | | Streptococcus | 6,606 | 2,118 | 12 | 66.178 | 26430 |
| | | | uberis | | | | | |
| | | | Rhodobacter | 4,494 | 1,441 | 12 | 45.020 | 159002 |
| | | | sphaeroides | | | | | |
| | | | Millerozyma | 3,851 | 1,235 | 12 | 38.579 | 27309 |
| | | | farinosa | | | | | |
| | | | Aspergillus oryzae | 269 | 86 | 12 | 2.695 | 40 |
| | | | Neospora caninum | 1,058 | 339 | 12 | 10.599 | 202 |
| | | | Cutibacterium | 9 | 3 | 12 | 0.090 | |
| | | | acnes | | | | | |
| | | | Thermus | 6 | 2 | 12 | 0.060 | |
| | | | scotoductus | | | | | |

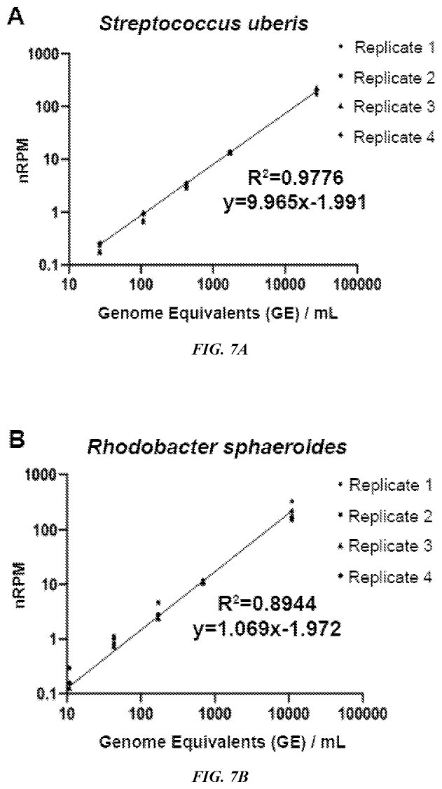

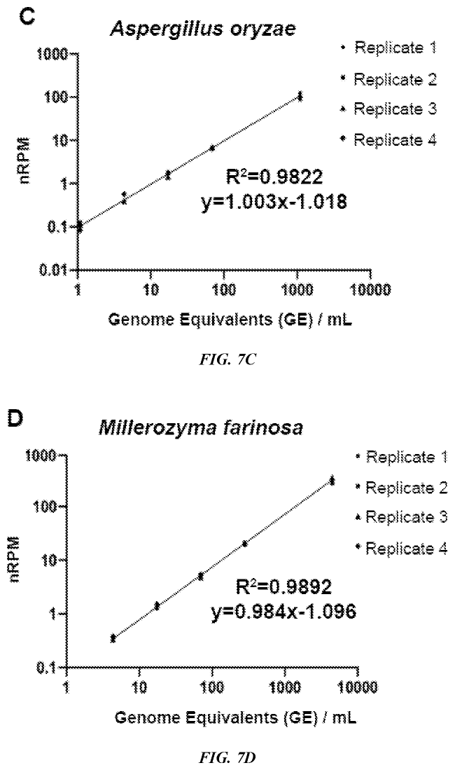

Limits of Detection and Linearity. To evaluate the limit of detections for bacteria and fungi for this assay, DNA was spiked from non-pathogenic microorganisms acquired from ATCC into healthy donor negative plasma in a series of 4-fold dilutions from 1:1 (no dilution) to 1:4096 (see Table 4). Each concentration of microorganism was tested on mNGS with 4 replicates for reproducibility. The bacteria and fungi tested include Streptococcus uberis, Rhodobacter sphaeroides, Millerozyma farinosa, and Aspergillus oryzae. Thresholds were chosen based on the nPRM corresponding to Youden's index on the training data ROC curve and using the composite standard. The bacterial nRPM thresholds were 2.6 and 0.54 for Illumina and nanopore sequencing, respectively; the fungal nRPM was 0.10 for Illumina and nanopore sequencing. The LoD was defined as the dilution at which mNGS testing detected the pathogen at levels above the nRPM threshold in 4 of 4 replicates. To evaluate assay linearity, a linear regression was performed on the same four sets of 615 serially diluted positive controls used in the LoD. The nRPM values were plotted against the input concentration (copies, or genome equivalents per mL). The best fit regression line along with the linear equation and R2 value was added to the plotted values (see FIG. 7).

TABLE 4
Fungal true positives (TP), false positives (FP), false
negatives (FN) using Illumina and nanopore sequencing:
Illumina sequencing - Gold Standard

| | Sample | Training or | Pathogen | | Normalized | % of all | # | NGS |
|---|---|---|---|---|---|---|---|---|
| Case | Type | Validation | Species | RPM | RPM | Microbes | Reads | call |
| S58 | Pleural | Validation | Aspergillus | 6.82 | 4.82 | 0.22 | 237 | TP |
| | Fluid | | fumigatus | | | | | |
| S26 | Pleural | Training | Candida | 0.00 | 0.00 | 0.00 | 0 | FN |
| | Fluid | | albicans | | | | | |
| S27 | Abscess | Validation | Candida | 25.26 | 25.26 | 0.28 | 161 | TP |
| | | | glabrata | | | | | |
| S27 | Abscess | Validation | Candida | 9.57 | 9.57 | 0.11 | 61 | FP |
| | | | albicans | | | | | |
| S29 | Abscess | Training | Candida | 0.00 | 0.00 | 0.00 | 0 | FN |
| | | | albicans | | | | | |
| S37 | Perihepatic | Training | Candida | 0.37 | 0.74 | 0.00 | 2 | FN |
| | Fluid | | krusei | | | | | |
| S49 | Swab | Training | Candida | 130.07 | 65.03 | 0.08 | 563 | TP |
| | | | albicans | | | | | |
| S51 | BAL | Training | Aspergillus | 1.06 | 4.22 | 0.07 | 8 | TP |
| | | | fumigatus | | | | | |
| S72 | Peritoneal | Training | Candida | 1.59 | 9.02 | 0.65 | 15 | TP |
| | Fluid | | albicans | | | | | |
| S77 | Peritoneal | Training | Candida | 0.13 | 0.72 | 0.03 | 1 | FN |
| | Fluid | | albicans | | | | | |
| S59 | Peritoneal | Validation | Candida | 504.19 | 178.26 | 0.08 | 3585 | TP |
| | Fluid | | glabrata | | | | | |
| S59 | Peritoneal | Validation | Pichia | 0.42 | 0.15 | 0.00 | 3 | FP |
| | Fluid | | kluyveri | | | | | |
| S59 | Peritoneal | Validation | Candida | 0.14 | 0.03 | 0.00 | 1 | FN |
| | Fluid | | krusei | | | | | |
| S79 | Chest mass | Validation | Coccidioides | 83.77 | 41.89 | 0.44 | 341 | TP |
| | | | immitis | | | | | |
| S80 | Chest mass | Training | Coccidioides | 2.97 | 2.10 | 0.23 | 15 | TP |
| | fluid | | immitis | | | | | |
| S83 | BAL | Validation | Aspergillus | 9.50 | 1.19 | 0.52 | 214 | TP |
| | | | terreus | | | | | |
| S83 | BAL | Validation | Aspergillus | 2.98 | 0.19 | 0.16 | 67 | TP |
| | | | fumigatus | | | | | |
| S85 | Abscess | Validation | Talaromyces | 0.11 | 0.11 | 0.01 | 3 | FP |
| | | | marneffei | | | | | |
| S4 | CSF | Validation | Candida | 28.97 | 1.81 | 0.77 | 10 | TP |
| | | | parapsilosis | | | | | |
| S5 | Joint Fluid | Validation | <A series of | n/a | n/a | n/a | n/a | FP |
| | | | fungi that is | | | | | |
| | | | cross-over from | | | | | |
| | | | a high titer of | | | | | |
| | | | Saccharomyces | | | | | |
| | | | cerevisiae, which | | | | | |
| | | | was determined | | | | | |
| | | | to be clinically | | | | | |
| | | | non- | | | | | |
| | | | pathogenic> | | | | | |
| S22 | Peritoneal | Validation | Candida | 18.26 | 584.34 | 0.20 | 9 | TP |
| | Fluid | | albicans | | | | | |
| S22 | Peritoneal | Validation | Candida | 16.23 | 519.41 | 0.17 | 8 | TP |
| | Fluid | | glabrata | | | | | |
| S127 | CSF | Validation | Coccidioides | 88.27 | 15.60 | 0.49 | 444 | TP |
| | | | immitis | | | | | |
| S127 | CSF | Validation | Blastomyces | 0.60 | 0.11 | 0.00 | 3 | FP |
| | | | dermatitidis | | | | | |
| S167 | Peritoneal | Validation | Lodderomyces | 0.14 | 0.14 | 0.03 | 1 | FP |
| | Fluid | | elongisporus | | | | | |
| S3 | Joint Fluid | Validation | Lodderomyces | 0.16 | 0.44 | 0.00 | 1 | FP |
| | | | elongisporus | | | | | |
| S79 | Chest mass | Validation | Aspergillus | 0.25 | 0.12 | 0.00 | 1 | FP |
| | | | fumigatus | | | | | |
| S94 | CSF | Validation | Coccidioides | 64.42 | 0.50 | 0.58 | 546 | TP |
| | | | immitis | | | | | |
| S116 | BAL | Validation | Cryptococcus | 116.75 | 3.65 | 0.33 | 986 | TP |
| | | | neoformans | | | | | |
| S121 | CSF | Validation | Coccidioides | 437.20 | 38.64 | 0.49 | 4799 | TP |
| | | | immitis | | | | | |
| S141 | Perigastric | Validation | Candida | 0.00 | 0.00 | 0.00 | 0 | FN |
| | Fluid | | parapsilosis | | | | | |
| S142 | Abscess | Validation | Candida | 4.40 | 1.56 | 0.12 | 3 | TP |
| | | | tropicalis | | | | | |
| S97 | CSF | Validation | Candida | 9.57 | 9.57 | 0.44 | 60 | TP |
| | | | glabrata | | | | | |
| S101 | Back Fluid | Validation | Candida | 0.29 | 0.50 | 0.06 | 2 | TP |
| | | | albicans | | | | | |
| S103 | CSF | Validation | Candida sp. | 0.14 | 0.19 | 0.00 | 1 | FP |
| | | | VVT-2012 | | | | | |
| S104 | Peritoneal | Validation | Candida | 10.28 | 10.28 | 0.57 | 78 | TP |
| | Fluid | | glabrata | | | | | |
| S105 | CSF | Validation | Cryptococcus | 4262.78 | 33.30 | 0.95 | 31493 | TP |
| | | | neoformans | | | | | |
| S106 | CSF | Validation | Candida | 22302.21 | 2787.78 | 0.99 | 163078 | TP |
| | | | parapsilosis | | | | | |
| S107 | CSF | Validation | Candida | 29.15 | 14.58 | 0.79 | 189 | TP |
| | | | parapsilosis | | | | | |
| S108 | CSF | Validation | Candida | 47.69 | 33.72 | 0.90 | 296 | TP |
| | | | parapsilosis | | | | | |
| S109 | CSF | Validation | Cryptococcus | 122.20 | 43.20 | 0.69 | 1214 | TP |
| | | | neoformans | | | | | |
| S110 | BAL | Validation | Aspergillus spp | 0.72 | 1.02 | 0.10 | 6 | TP |
| S110 | BAL | Validation | Aspergillus | 0.48 | 0.53 | 0.07 | 4 | FP |
| | | | oryzae | | | | | |
| S123 | CSF | Validation | Cryptococcus | 1.07 | 0.27 | 0.03 | 12 | TP |
| | | | gattii | | | | | |
| S153 | Peritoneal | Validation | Malassezia | 0.12 | 0.16 | 0.01 | 3 | FP |
| | Fluid | | globosa | | | | | |
| S114 | Abscess | Validation | Coccidioides | 81.02 | 114.58 | 0.30 | 839 | TP |
| | | | immitis | | | | | |
| S114 | Abscess | Validation | Penicillium | 0.77 | 1.09 | 0.00 | 8 | FP |
| | | | rubens | | | | | |
| S119 | Peritoneal | Validation | Candida sp. | 0.41 | 0.58 | 0.00 | 1 | FP |
| | Fluid | | VVT-2012 | | | | | |
| S120 | Urine | Validation | Candida | 15.60 | 31.20 | 0.01 | 65 | TP |
| | | | albicans | | | | | |
| S136 | BAL | Validation | Aspergillus | 21.85 | 2.73 | 0.70 | 62 | TP |
| | | | fumigatus | | | | | |
| S145 | Pleural | Validation | Tetrapisispora | 0.18 | 0.13 | 0.00 | 1 | FP |
| | Fluid | | blattae | | | | | |
| S124 | CSF | Validation | Coccidioides | 144.47 | 6.38 | 0.47 | 1467 | TP |
| | | | immitis | | | | | |
| S125 | BAL | Validation | Histoplasma | 3.44 | 0.02 | 0.08 | 16 | FN |
| | | | capsulatum | | | | | |
| S126 | BAL | Validation | Pneumocystis | 4991.82 | 19.50 | 0.79 | 13533 | TP |
| | | | jirovecii | | | | | |

Nanosequencing - Gold Standard

| | Sample | | Pathogen | | Normalized | % of all | # | NGS |
|---|---|---|---|---|---|---|---|---|
| Case | Type | | Species | RPM | RPM | Microbes | Reads | call |
| S4 | CSF | Validation | Candida | 8.70 | 0.54 | 0.26 | 10 | TP |
| | | | parapsilosis | | | | | |
| S22 | Peritoneal | Validation | Candida | 7.38 | 236.16 | 0.62 | 8 | TP |
| | Fluid | | glabrata | | | | | |
| S22 | Peritoneal | Validation | Candida | 1.85 | 59.04 | 0.15 | 2 | TP |
| | Fluid | | albicans | | | | | |
| S58 | Pleural | Validation | Aspergillus | 5.89 | 4.16 | 0.85 | 11 | TP |
| | Fluid | | fumigatus | | | | | |
| S83 | BAL | Validation | Aspergillus | 5.75 | 0.72 | 0.60 | 9 | TP |
| | | | terreus | | | | | |
| S83 | BAL | Validation | Aspergillus | 1.92 | 0.13 | 0.20 | 3 | TP |
| | | | fumigatus | | | | | |
| S26 | Pleural | Training | Candida | 0.00 | 0.00 | 0.00 | 0 | FN |
| | Fluid | | albicans | | | | | |
| S29 | Abscess | Training | Candida | 0.00 | 0.00 | 0.00 | 0 | FN |
| | | | albicans | | | | | |
| S37 | Perihepatic | Training | Candida | 0.00 | 0.00 | 0.00 | 0 | FN |
| | fluid | | krusei | | | | | |
| S51 | BAL | Training | Aspergillus | 0.00 | 0.00 | 0.00 | 0 | FN |
| | | | fumigatus | | | | | |
| S72 | Peritoneal | Training | Candida | 1.97 | 11.16 | 1.00 | 11 | TP |
| | Fluid | | albicans | | | | | |
| S77 | Peritoneal | Training | Candida | 0.00 | 0.00 | 0.00 | 0 | FN |
| | Fluid | | albicans | | | | | |
| S80 | Chest | Training | Coccidioides | 0.97 | 0.69 | 0.50 | 1 | TP |
| | mass fluid | | immitis | | | | | |
| S27 | Abscess | Validation | Candida | 4.72 | 4.72 | 0.83 | 5 | TP |
| | | | glabrata | | | | | |
| S49 | Swab | Validation | Candida | 108.50 | 54.25 | 0.13 | 138 | TP |
| | | | albicans | | | | | |
| S59 | Peritoneal | Validation | Candida | 116.44 | 41.17 | 0.02 | 174 | TP |
| | Fluid | | glabrata | | | | | |
| S59 | Peritoneal | Validation | Candida | 5.35 | 0.00 | 0.00 | 8 | FN |
| | Fluid | | krusei | | | | | |
| S79 | Chest | Validation | Coccidioides | 60.14 | 30.07 | 0.69 | 101 | TP |
| | mass | | immitis | | | | | |

Bioinformatics analysis. Illumina sequencing data were analyzed for pathogens using the clinically validated SURPI+(sequence based ultra-rapid pathogen identification) computational pipeline v1.0.63-dev 7,20,58. SURPI+ uses the entirety of the NCBI GenBank nt database (March 2015 distribution) as the reference database and incorporates taxonomic classification algorithms for accurate identification of pathogens as described in Miller et al. (Genome Res 29:831-842 (2019)). Nanopore sequencing data were analyzed using SURPIrt (SURPI “real-time”) software (SURPIrt research 1.0.14-build.86). Raw fast5 files were base called using MinKNOW software v3.1.20 installed on the GridION in real-time mode without polishing. The base called reads were run through in-house developed scripts for sample demultiplexing using the BLASTn (v2.7.1+) aligner at a significance E-value threshold of 10-2. After trimming adapters and removing low-quality and low-complexity sequences, the first 450 nt of the preprocessed read was partitioned into three 150 nt segments, followed by rapid low-stringency identification of candidate pathogen reads using SNAP (version 1.0dev100) alignment to microbial reference databases (viral portion of 2019 NCBI nt; bacterial RefSeq; fungal and parasitic pathogens in the fungal RefSeq and parasitic RefSeq databases), using an edit distance of 5059. Candidate reads were then filtered and taxonomically classified as described in Miller et al. Real-time analysis was performed by running the SURPIrt pipeline in continuously looping mode, with ˜100k-200k nanopore reads analyzed per batch.

Computational algorithm for pathogen identification. A pathogen identification algorithm that was applicable for both Illumina and 642 nanopore datasets outputted by SURPI or SURPIrt (see above) was developed to assess and optimize performance accuracy. An initial reference database was manually tabulated based on pathogens detected in body fluids by culture and/or PCR testing. The algorithm calculated a nRPM pathogen count, filtered out taxonomically related microorganisms, and defined criteria for pathogen detection, as explained in detail below.

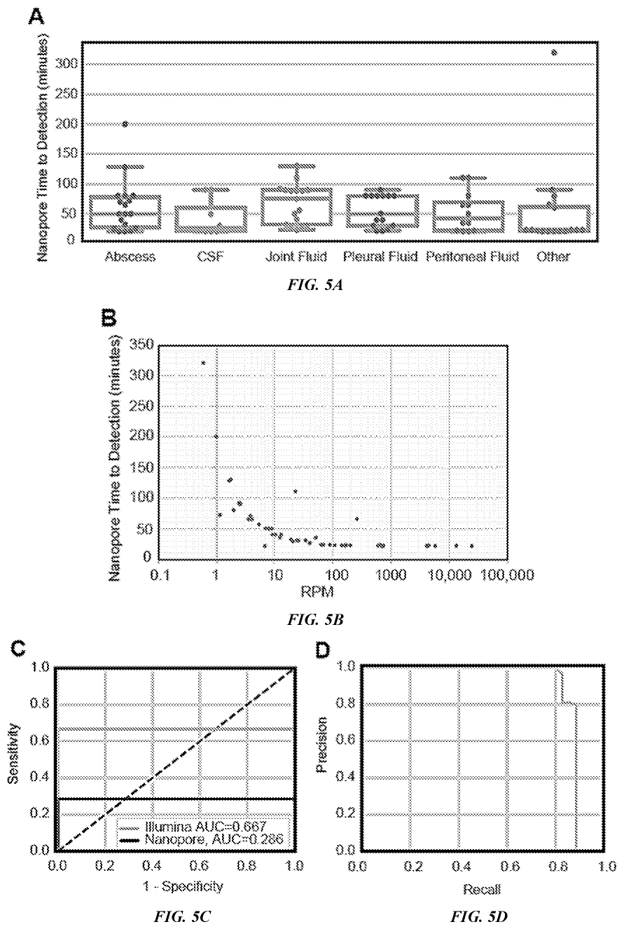

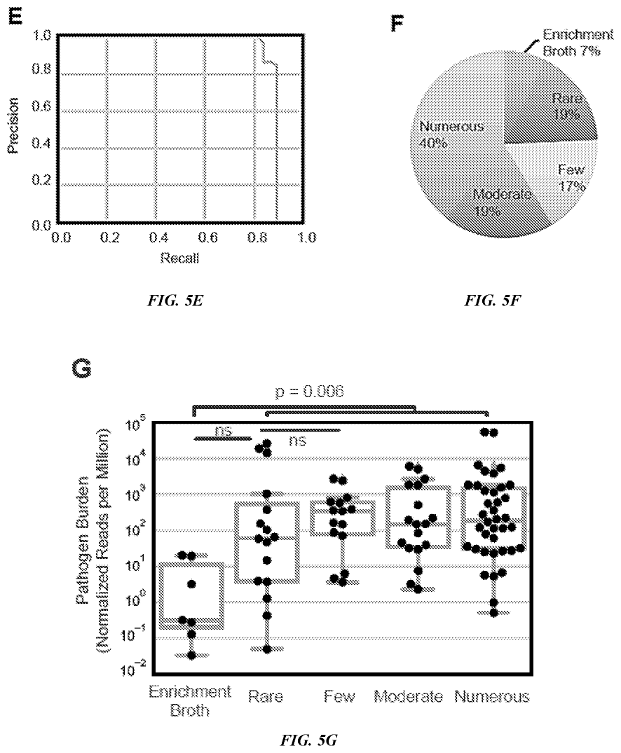

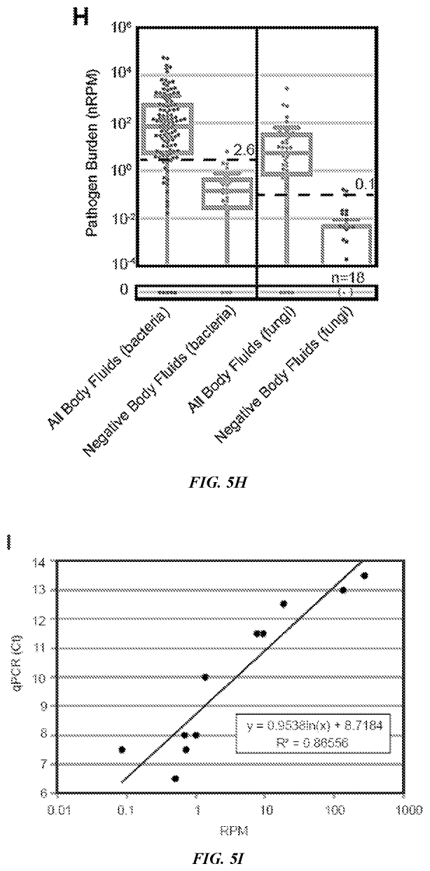

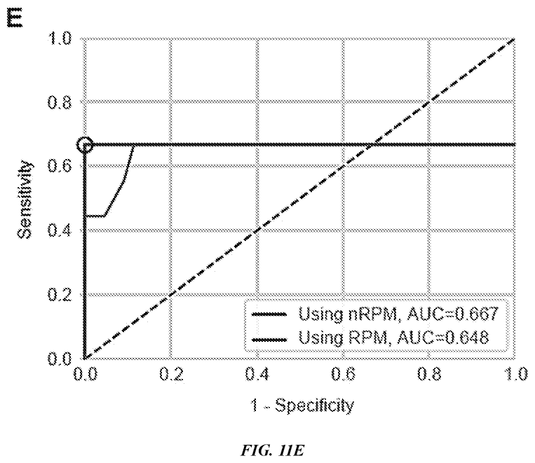

(1) Calculating a normalized RPM. A nRPM metric was developed to standardize microorganisms across samples with uneven sequencing depths and input DNA concentrations. For Illumina sequencing, the RPM was defined as the number of pathogen reads divided by the number of preprocessed reads (reads remaining after adapter trimming, low-quality filtering, and low-complexity filtering), while for nanopore sequencing, the RPM was defined as the number of pathogen reads divided by the number of base called reads. A nRPM was calculated that adjusted the RPM with respect to background based on the Ct value (to the nearest 0.5 increment) during the PCR amplification step of library preparation. As the average Ct value across all samples was 7, the nRPM was defined as nRPM=RPM/2 (Ct-7). Receiver-operating characteristic (ROC) and precision-recall curves were plotted using the Python software package and pandas data analysis library. The optimal nRPM threshold was obtained by plotting the ROC curve at varying nRPM values and determining the nRPM at Youden's Index. The incorporation of a nRPM metric is based on a previous observation of a log-linear relationship between the qPCR Ct value and the RPM of representative, presumed background contaminant microorganisms such as Achromobacter xylosoxidans (see FIG. 5I). Thus, assuming a constant background level of Achromobacter xylosoxivdans, measured RPM would be inversely correlated with the input concentration. In the current study, better performance was achieved using the nRPM versus the RPM metric (see FIGS. 11B and E).

(2) Filtering out closely-related microorganisms. Taxonomic classification using metagenomics data commonly yields a minority fraction of reads that map to related taxa with the same family or genus as the microorganism truly present in the sample. In order to minimize cross-species misalignments for closely related microorganisms, the nRPM of microorganisms that share a genus or family designation was penalized (reduced). A penalty of 10% and 5% was used for genus and family respectively, based 675 on the empirical maximization of specificity from the ROC curve of the training set. For example, 676 if Escherichia coli had an nRPM of 100 and Shigella sonnei (from same Enterobacteriaceae 677 family) had an nRPM of 5, the nRPM of Shigella sonnei would be reduced to zero. In the current study, better performance was achieved in the training dataset using this filter.

(3) Criteria for pathogen detection. Two criteria were developed for pathogen detection. The candidate pathogen was required to 683 (i) have a minimum number of pathogen-specific reads identified (≥3 for bacteria and >1 for fungi) (see FIGS. 11A and D), and (ii) meet an optimal nRPM threshold. Optimal nRPM thresholds using composite standards were set to the maximum Youden's index (bacterial nRPM of 2.6 and 0.54 for Illumina and nanopore sequencing, respectively; fungal RPM of 0.10 and 0.10 for Illumina and nanopore sequencing, respectively), as determined from the ROC curve of the training set. The clinical gold standard (culture/16S PCR) used the same thresholds except that the bacterial nRPM threshold for Illumina sequencing was 3.2.

Statistical methods. To evaluate accuracy, two criteria were applied: (i) a clinical gold standard based on culture and 16S PCR results obtained through routine clinical care, and (ii) a composite standard based on a combination of clinical testing (culture and 16S/28S-ITS PCR), orthogonal testing (e.g., digital PCR, serology), and clinical adjudication. The specific scoring algorithm is outlined as follows (see Table 5): Based on the clinical or composite standard, true positives (TP) or false negatives (FN) were scored for each microorganism that was detected or not detected by mNGS, respectively. For each sample, a true-negative (TN) was scored if no other microorganism(s) other than the expected ones based on the clinical or composite standard were detected by mNGS; otherwise, a false-positive (FP) was scored. Multiple FP results in a sample were counted as one FP overall. p-values were calculated using a two-sided Welch's t-test at a significance p-value threshold of 0.05. All data points in the study were performed once, except the LoD studies which were performed in four replicates at each dilution.

TABLE 5
Scoring system for the mNGS accuracy evaluation.

| Gold Standard | mNGS | TP/FN Score | TN/FB Score |
|---|---|---|---|
| Negative | Negative | N/A | 1 TN for all organisms |
| | | | not detected |
| Negative | Positive for | N/A | 1 FP for the organism(s) |
| | any organism(s) | | found on mNGS |
| Positive for | Positive for the | 1 TP for that | 1 TN for all other |
| 1 organism | identical organism | organism | organisms not detected. |
| Positive for | Positive for a | 1 FN for organism | 1 FP for different organism |
| 1 organism | different organism | detected by the | detected by mNGS |
| | | gold standard | |
| Positive for | Positive for only | 1 TP for the | 1 TN for all other |
| 2 organisms | 1 organism | organism detected | organisms not detected. |
| | | AND 1 FN for the | |
| | | organism not detected. | |
| Positive for | Positive for 2 | 2 TP for the two | 1 FP for different organisms |
| 3 organisms | of 3 organisms | organisms detected | detected by mNGS |
| | and positive for | AND 1 FN for the | |
| | 2 different organisms | organism not detected | |

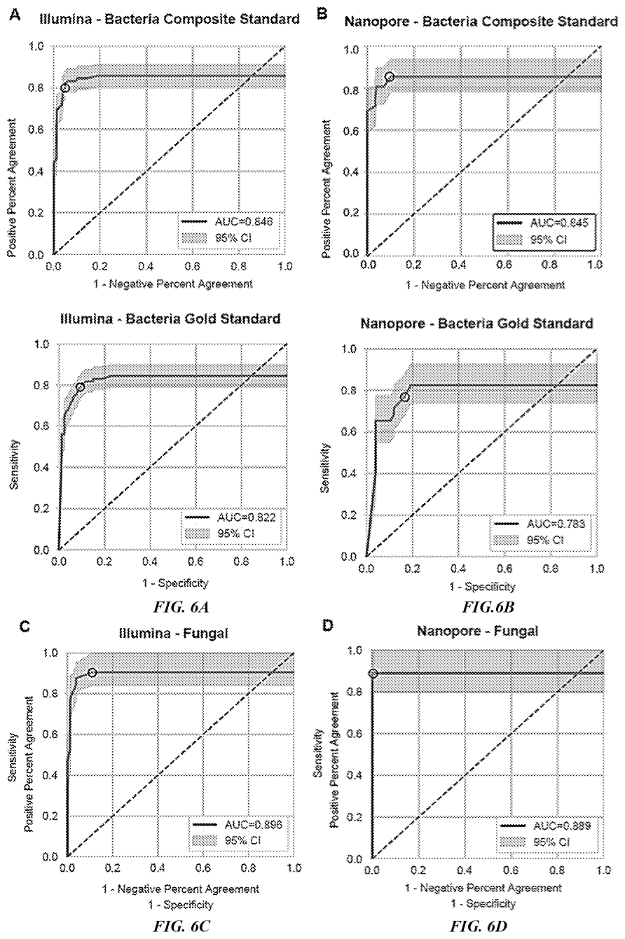

Confidence intervals for the ROC curves. To evaluate the reliability of the validation set data, a custom python script was coded that bootstrapped the dataset by randomly resampled the dataset with replacement to generate a replicate dataset of the same size for 2000 iterations. The resultant distribution was used to produce a 95% confidence interval (CI) for the ROC curve (see FIG. 6).

Orthogonal confirmation of mNGS results. Digital PCR (dPCR) for orthogonal confirmation of mNGS results was performed using the Biorad QX200 Droplet Digital PCR System. The advantages of dPCR include the ability for absolute quantification, improved detection of very low-abundance nucleic acids with high precision, and higher tolerance to the presence of inhibitors and/or contaminants in the body fluid samples. Thus, the use of dPCR was deemed to be a more robust indicator for the presence of pathogen-specific DNA in the body fluids than conventional PCR. All primer and probe pairs were synthesized by Integrated DNA Technologies, Inc. and first validated using positive control microorganisms (see Table 6). Genomic DNA from positive control microorganisms was purchased from ATCC and mechanically sheared (MiniTUBE, Covaris) to an average of 200-300 base pairs. For Sanger sequencing, DNA was first cloned into colonies using a TOPO TA Cloning Kit (ThermoFisher). Sanger sequencing of the clones was then performed at Elim Biopharmaceuticals, Inc. Sequencing traces were analyzed on Geneious software (version 10.2.3) and aligned to the National Center for Technology Information nt database using BLAST. Serology confirmation of the Bartonella case was performed by Quest Diagnostics.

TABLE 6
PCR primers used for orthogonal validation. (SEQ ID Nos: 197-225)

| | | | | | | Predicted |
|---|---|---|---|---|---|---|
| | | | | | | Amplicon |
| | | | | | | Length |
| Name | Organism | Assay | Primer 1 | Primer 2 | Probe | (bp) |
| [symbol] | [symbol] | digital PCR | AATATCGTGCAGCGAGGTG | CCGGCATCCACCATATTCT | AACGGCACGTCTTCGACTT | 59 |
| | [symbol] | | | | | |
| 1-Saccharomcyes | Saccharomyces | digital PCR | ACGCTGAGGCTTTCAAGAAT | TTTGTTGGCGTAAACAGTCG | CTCAGAAGAGGTTTCACAACGA | 67 |
| Cerevisiae | cerevisiae | | | | | |
| [symbol] | Streptococcus | digital PCR | AGATAATGGCACAGCCAATCA | TATTTACGGGCACATCATCG | CGATGCCGATATAGTTGTAGATAGTC | 68 |
| | [symbol] | | | | | |
| [symbol] | [symbol] | digital PCR | CCGACAGATCCTTGTCCAAC | GGGTGAAAAACGCCAACTC | GTTGGCCGTTGAGCCATAC | 60 |
| | [symbol] | | | | | |
| [symbol] | [symbol] | digital PCR | CAACTGCATTTAACATATCAACAGG | AAATGCCCAGAAGAAAAACT | TTCTCCAACTCCCATTAAATATCT | 70 |
| | [symbol] | | | | | |
| [symbol] | [symbol] | digital PCR | GATGGCTCGCCACTTTAGAA | CCTAATGTTTGCATGCCGTTA | TGAACCGCGTCATTTATTATTT | 69 |
| | [symbol] | | | | | |
| [symbol] | [symbol] | digital PCR | GCGAAACCCAGCAGAAACT | GAGTACGTTACCGCTATATTCACG | TACCTTTATGGCCCTGCTG | 68 |
| | [symbol] | | | | | |
| | [symbol] | | | | | |
| 1-Ecoli | Escherichia | digital PCR | GGCGATTTTCGGTCTGACT | CGTCGCGCGTATAATGTG | CGTGGGGTGAACGCTAAC | 55 |
| | coli | | | | | |
| [symbol] | [symbol] | [symbol] | TATCGCCAACCAGGATGC | CCGCCAGGTAAGCATTGA | Not used | 65 |
| | [symbol] | sequencing | | | | |
| [symbol] | [symbol] | digital PCR | AGCGATGCGTATTGTTCTTG | ATATCCCATAATCGGGCAGA | CCGATGAAATATCGCCTGAT | 60 |
| | [symbol] | | | | | |

[symbol]

indicates data missing or illegible when filed

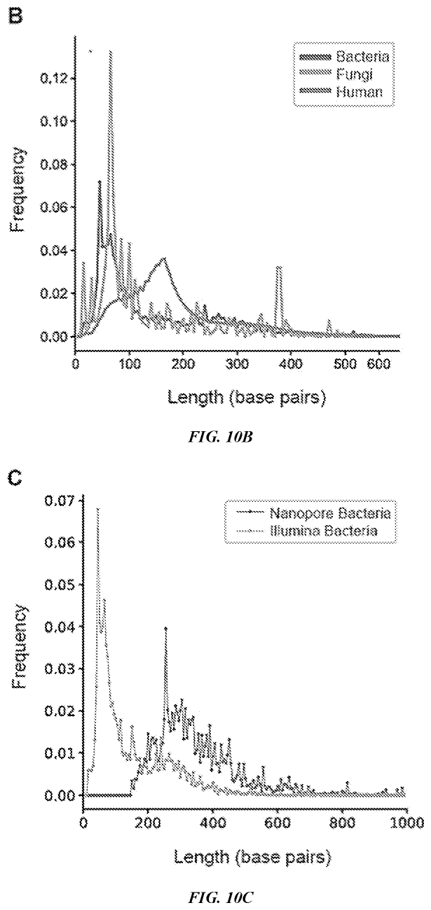

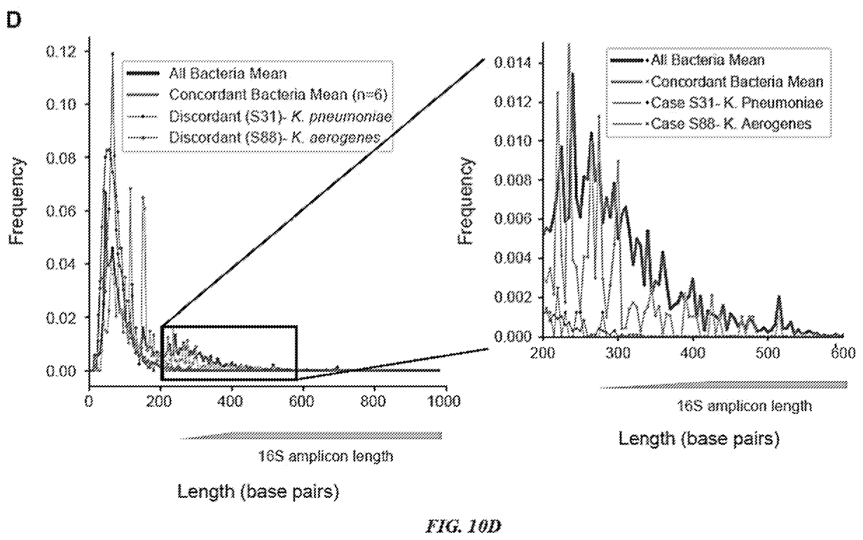

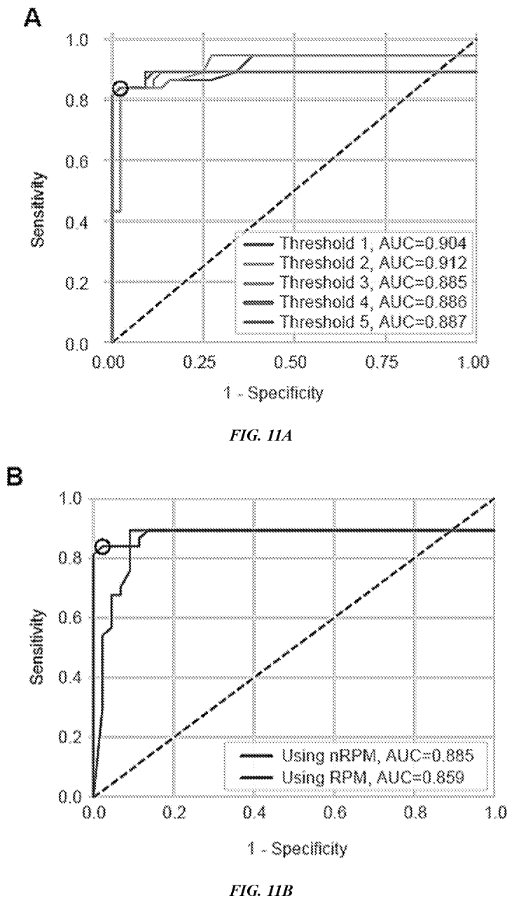

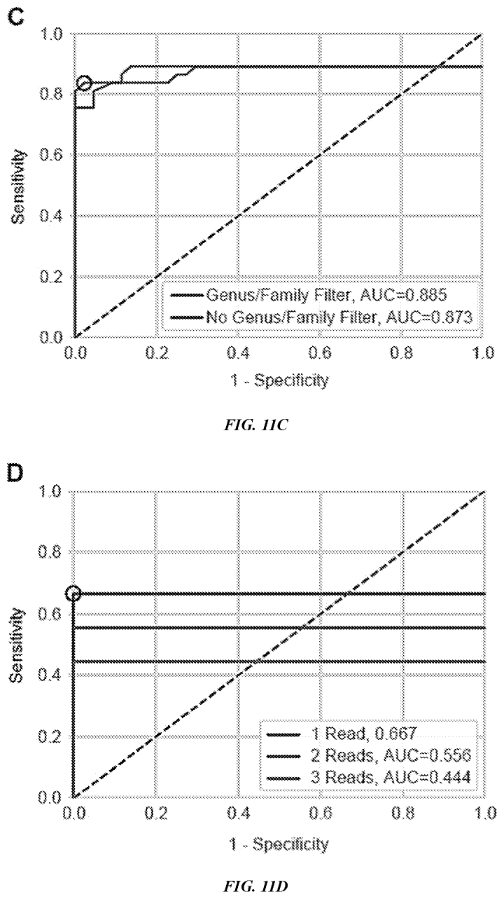

Analysis of pathogen and human DNA lengths. Pathogen-specific length distributions in mNGS data were obtained by aligning paired-end Illumina reads or single-end nanopore reads to individual pathogen genomes (see FIG. 10). For Illumina sequencing data, unaligned, human-depleted FASTQ reads were extracted using the bamtofastq function in the bedtools software package, followed by alignment to species-specific microbial reference genomes using BWA. An in-house developed Python program and Linux shell scripts were used to extract read lengths from resultant paired-end SAM files. For nanopore sequencing data, read lengths were directly extracted from SAM-formatted pathogen reads outputted from the SURPIrt pipeline. Histograms of the read lengths were plotted using the software package Matplotlib as implemented in Python.

For characterization of human DNA length distributions from Illumina data, FASTQ files were first trimmed for Illumina adapters with cutadapt (v1.16), followed by alignment with BWA 742 (v0.7.12) to the hg38 human reference genome. This revealed a previously described peak of ˜160 nt that corresponds to nuclear DNA wrapped around a single histone (see FIG. 10A).

Length distributions were assessed from 58 bacterial and 10 fungal pathogens by histogram analysis, with the inclusion criteria of at least 10 paired-end reads aligned to each pathogen genome (see FIG. 10B). The average distribution skewed towards shorter length fragments with a long tail extending to −700 nt, and no significant size differences between bacterial and fungal DNA were observed. This range of pathogen DNA sizes was similar to what had been previously observed in plasma and urine. Bacterial length distributions from nanopore sequencing were longer on average (356 nt) than from Illumina sequencing (177 nt) (see FIG. 10C).

Data availability. Metagenomic sequencing data (FASTQ files) after removal of human genomic reads have been deposited into the NCBI Sequence Read Archive (SRA) (PRJNA558701, under umbrella project PRJNA234047).

Software and code accessibility. SURPI+v1.0 (github.com/chiulab/SURPI-plus-dist) and SURPIrt v1.0 software 761 (github.com/chiulab/SURPIrt-dist) have been deposited on GitHub and are available for download for research use only. Linux (Ubuntu 16.04.6) and Python (python 2.7.12) scripts used for construction of dual-use Illumina and nanopore barcodes are provided below. Other custom scripts for ROC curve and read length analysis have been deposited on Github (github.com/wei2gu/2020-NGSInfectedBodyFluids/).

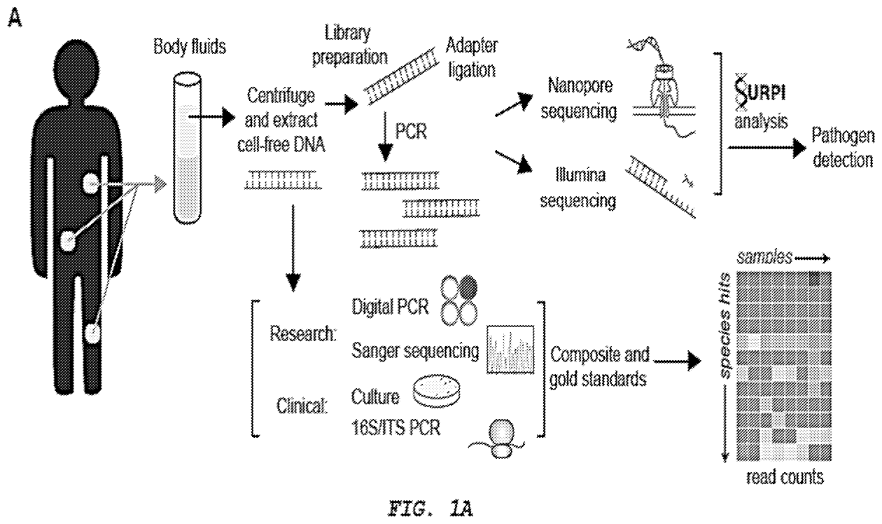

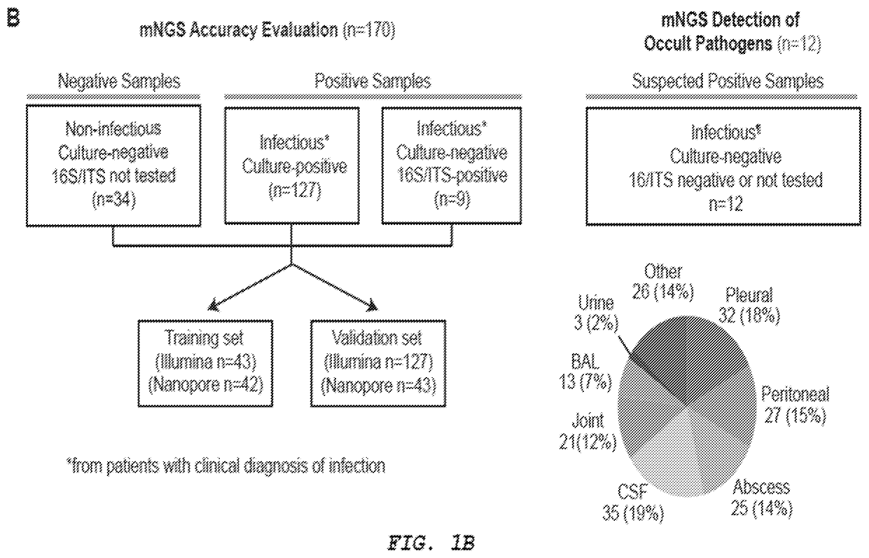

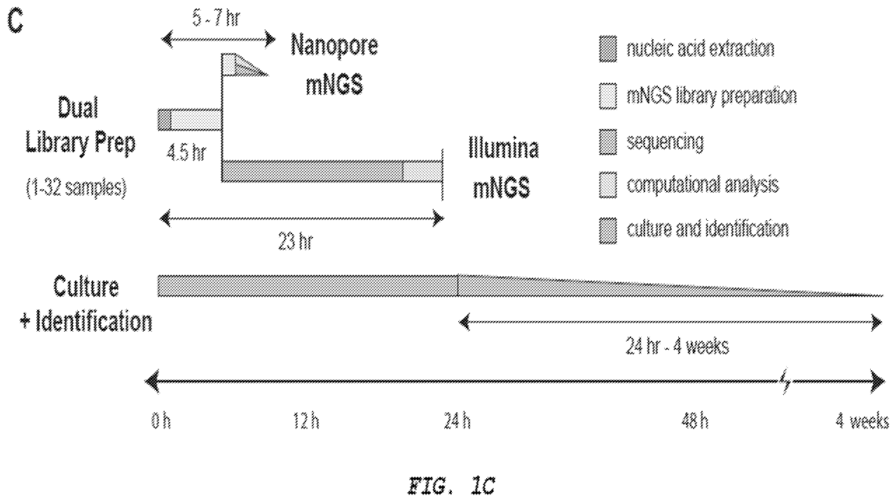

Sample Collection. A total of 182 body fluid samples from 160 patients, including 25 abscess, 21 joint, 32 pleural, 27 peritoneal, 35 cerebrospinal, 13 bronchoalveolar lavage (BAL), and 29 other body fluids (see Table 7 and Table 1), were collected as residual samples after routine clinical testing in the microbiology laboratory. Among the 182 samples, 170 were used to evaluate the accuracy of mNGS testing by Illumina sequencing (see FIG. 1A, and Table 1). These accuracy samples included 127 positives by culture (with pathogen(s) identified to genus or species level), 9 culture-negative but positive by 16S or 28S-ITS PCR, and 34 negative controls from patients with alternative non-infectious diagnoses (e.g., cancer, trauma) (see FIG. 1B). Out of the 170 samples used for evaluation of accuracy, the first 87 consecutively collected samples were used to compare the accuracy of nanopore relative to Illumina sequencing. The remaining 12 body fluid samples out of 182 total were collected from patients with negative direct microbiological testing of the body fluid but highly suspected or orthogonally proven infection, as described in the case series section below (see FIG. 1B, Table 11, and Clinical Vignettes in the Examples). These 12 body fluids were analyzed to demonstrate the diagnostic utility of mNGS testing for detecting pathogens in cases of unknown infectious etiology. Negative external controls (pooled donor plasma matrix) and positive external controls (donor plasma matrix spiked with known quantities of DNA from organisms considered non-pathogenic to humans) were run in parallel with body fluid samples (see Table 3).

Study Patients. Among 158 patients out of 160 with available clinical data, 144 (91%) were hospitalized, of whom 61 (39%) required intensive care unit (ICU) management and 45 (28%) met clinical criteria for sepsis (32%) were immunocompromised due to organ transplantation, recent chemotherapy, human immunodeficiency virus (HIV) infection, or drug-induced immunosuppression, and 71 (45%) were on antibiotics at the time of body fluid collection (Table 7). According to usual standard-of-care practices, bacterial cultures were obtained for all body fluids, with 63 (35%) and 81 (45%) having additional cultures done for acid-fast bacilli (AFB) and fungi, respectively.

TABLE 7
Patient and Sample Characteristics

| Patient Demographics (n = 158) | | |
|---|---|---|
| Age - years | | |
| Median (interquartile range) | 54 | (34-65) |
| Range | (0-92) | |
| Gender | | |
| Female - no. (%) | 75 | (47%) |
| Male - no. (%) | 83 | (53%) |
| Hospitalization | | |
| Patients Total - no. (%) | 158 | (100%) |
| In hospital | 144 | (91%) |
| In intensive care unit | 61 | (39%) |
| Days Hospitalized - no. (IQR) | 14 | (7-26) |
| 30-day mortality - no. (%) | 9 | (6%) |
| Immunocompromised - no. (%) | 51 | (32%) |
| On empiric antibiotics at time of body | 71 | (45%) |
| fluid collection - no. (%) | | |
| Sepsis according to SIRS criteria (>2) - | 45 | (28%) |
| no. (%)^{a} | | |
| Presumed Illness - no. (%) | | |
| Septic arthritis | 21 | (13%) |
| Respiratory infection | 39 | (25%) |
| Gastrointestinal abscess | 15 | (9%) |
| Soft Tissue abscess | 18 | (11%) |
| Peritonitis | 26 | (16%) |
| CNS infection | 32 | (20%) |
| Urinary Tract Infection | 3 | (2%) |
| Eye Infection | 1 | (0.6%) |
| Other | 3 | (2%) |
| Sample Characteristics (n = 182) | | |
| Sample Type - no. (%) | | |
| Abscess | 25 | (14%) |
| Cerebrospinal Fluid | 35 | (19%) |
| Joint Fluid | 21 | (12%) |
| Peritoneal Fluid | 27 | (14%) |
| Pleural Fluid | 32 | (18%) |
| Bronchoalveolar Lavage | 13 | (7%) |
| Other^{b} | 29 | (16%) |
| WBC count of body fluid - | 963 | (161-11,925) |
| median 10⁶/L (interquartile range) | | |
| Range | 1-382,000 | |
| Time to Final Culture Result - | 4.8 | (3.8-14.0) |
| median days (interquartile range) | | |
| Range - 10⁶/L | 1.3-35.7 | |
| Organism cultured - no. (%) | | |
| Staphylococcus aureus | 40 | (22%) |
| Streptococcus sp. | 15 | (8%) |
| Enterococcus sp. | 10 | (5%) |
| Gram Negative Rods | 30 | (15%) |
| Fungi | 46 | (23%) |
| Other | 20 | (10%) |
| Negative | 35 | (18%) |

^{a}SIRS, systemic inflammatory response syndrome
^{b}vitreous fluid, perihepatic fluid, surgical swab, subgaleal fluid, heel fluid swab, peri-graft fluid swab, anterior mediastinal fluid, chest fluid, chest wall mass, wound swab, synovial fluid, breast fluid, back fluid, fine needle aspirate (FNA), left thigh bursal fluid, peri-gastric fluid, thoracic spine seroma, peri-tonsillar drainage, knee swab, ililpsoas collection fluid, iliac wing fluid, retrogastric fluid, and urine

Metagenomic Sequencing Analysis. A dual-use barcoding protocol was developed for mNGS testing that was cross-compatible on both nanopore and Illumina sequencing platforms, suitable for all body fluids, and automated in the clinical microbiology laboratory on liquid handling workstations. The amount of input DNA varied over 6 logs from approximately 100 pg/mL in low cellularity fluids such as CSF to 100 μg/mL in purulent fluids. The median read depths for Illumina and nanopore sequencing were 7.2M (interquartile range or IQR 4.0-8.3M, range 0.15-35M) and 1.1M (IQR 1.0-1.5M, 137 range 0.29-6.7M), respectively (see Table 1). Metagenomic analysis for pathogen detection from Illumina data was performed using clinically validated SURPI+ software. Nanopore sequencing yielded 1 million reads per hour on average, with real-time data analysis performed using SURPIrt software, a new in-house developed bioinformatics pipeline for pathogen detection from metagenomic nanopore sequence data. After a 5-hour library preparation, nanopore sequencing detected pathogens in a median time of 50 minutes (IQR 23 143-80 minutes; range 21-320 minutes) (see FIG. 1C; and Table 1), with an overall sample-to-answer turnaround time of ˜6 hours, whereas the turnaround time for Illumina sequencing was ˜24 hours. The time to pathogen detection on the nanopore platform was independent of body fluid type (see FIG. 5A), but was inversely correlated with estimated pathogen DNA titers based on reads per million (RPM) (see FIG. 5B).

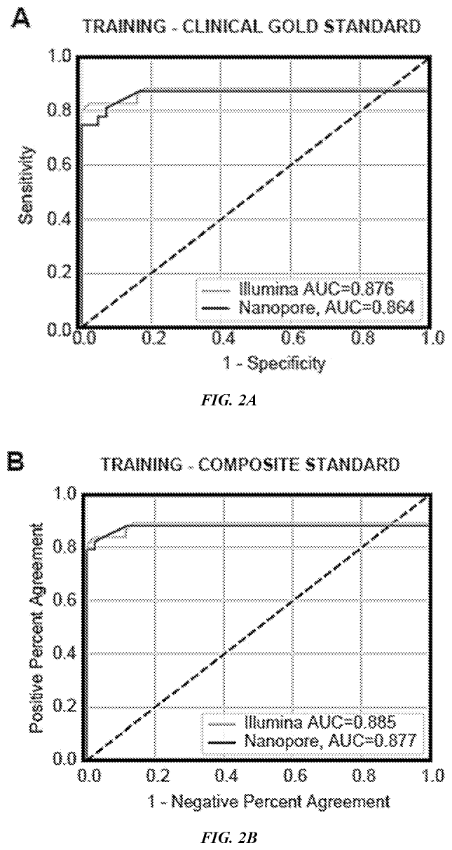

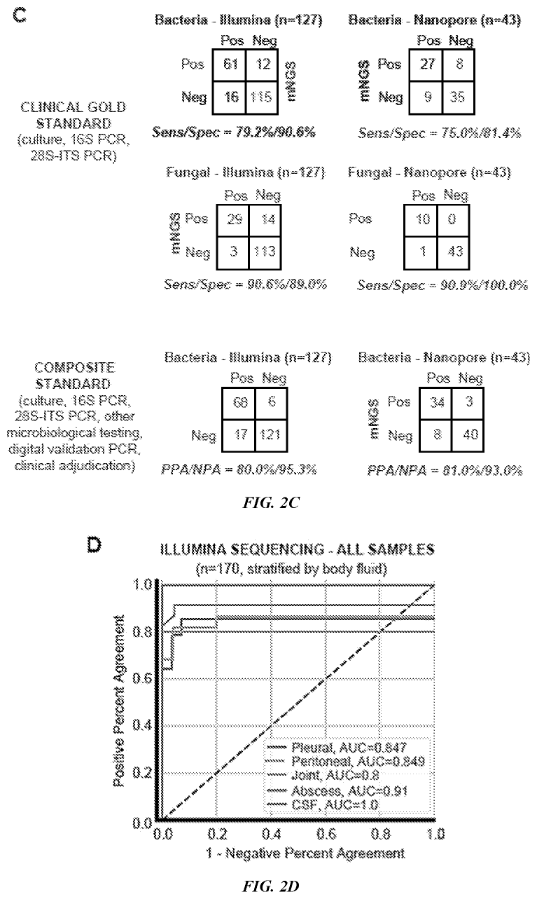

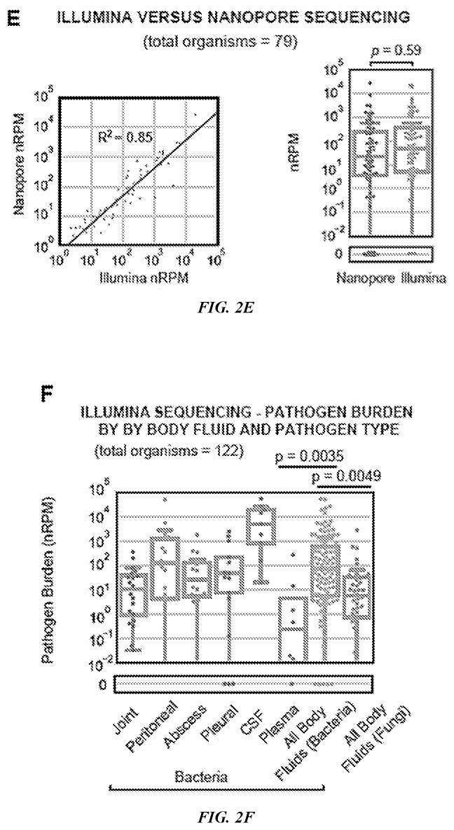

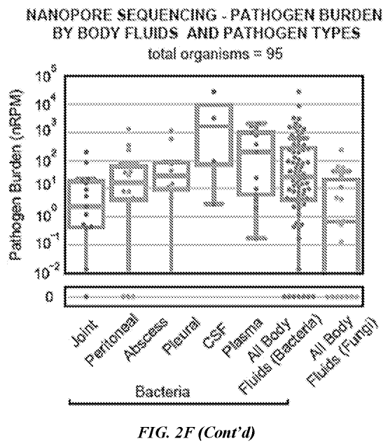

Test Accuracy. The accuracy evaluation focused on the performance of mNGS relative to gold standard culture and/or PCR testing for pathogen detection (see FIG. 1A). For bacterial pathogen detection, two reference standards were applied in the evaluation: a clinical gold standard consisting of available culture and 16S PCR results and a composite standard that incorporated additional results from: (i) orthogonal clinical testing of other sample types collected concurrently from the same patient, (ii) confirmatory research-based digital PCR (dPCR) testing, and (iii) 156 adjudication independently by an infectious disease specialist (CYC) and clinical pathologist (WG). Adjudication was performed after mNGS results were available by integrating all sources of information, including longitudinal patient chart review and dPCR testing (see FIG. 1A). Clinical samples were randomly divided into a training set (n=43 samples, 36 bacterial organisms, 8 fungal organisms) and validation set (n=127 samples, 85 bacteria, 32 fungi) for Illumina sequencing; and training set (n=42 samples, 34 bacteria, 7 fungi) and validation set (n=43 samples, 43 bacteria, 11 fungi) for nanopore sequencing, respectively. Receiver operator characteristic (ROC) and precision-recall curves for the training set were generated relative to the clinical and composite standards (see FIG. 2A-B; FIG. 5C-E; and Table 8)). The curves were plotted using a normalized read per million (nRPM) metric that adjusts the RPM according to PCR cycle threshold.

TABLE 8
Differences between clinical gold standard and composite standards.

| | | | Clinical Gold | Composite | |
|---|---|---|---|---|---|
| | Sample | Training or | Standard* | Standards | |
| Case # | Type | Validation | (Bacteria only) | (Bacteria only) | Reason |
| S8 | BAL | Validation | Staphylococcus aureus | Staphylococcus aureus, | Past BAL culture has the |
| | | | | Pseudomonas aeruginosa | same organism found on NGS |
| S17 | Pleural | Validation | Serratia sp. SCBI | Serratia sp. SCBI | Past blood culture positive |
| | Fluid | | (marcescens) | (marcescens), | for same organism and |
| | | | | Enterococcus faecium | dPCR was positive |
| S19 | Pleural | Validation | Serratia sp. SCBI | Serratia sp. SCBI | Past blood culture was |
| | Fluid | | (marcescens) | (marcescens), | positive for the same |
| | | | | Enterococcus faecium | organism |
| S31 | Pleural | Validation | Streptococcus mitis | Klebsiella pneumoniae | Positive dPCR for K. pneumoniae. |
| | Fluid | | | | Negative dPCR for S. mitis in this |
| | | | | | sample and the contralateral |
| | | | | | pleural fluid, in concordance |
| | | | | | with Illumina and nanopore |
| | | | | | sequencing (no reads to this |
| | | | | | organism) |
| S50 | Peritoneal | Training | Escherichia coli | Escherichia coli, | Past peritoneal fluid was |
| | Fluid | | | Klebsiella pneumoniae | positive for the same organism |
| S57 | Peritoneal | Training | Enterococcus faecium | Enterococcus faecium, | Positive dPCR for this |
| | Fluid | | | Klebsiella pneumoniae | organism |
| S74 | Peritoneal | Validation | Staphylococcus aureus | Staphylococcus aureus, | Positive dPCR for the two |
| | Fluid | | | Enterococcus faecalis, | additional organisms |
| | | | | Escherichia coli | |
| S59 | Peritoneal | Validation | Pseudomonas aeruginosa, | Pseudomonas aeruginosa, | Past body fluid was |
| | Fluid | | Candida glabrata, | Candida glabrata, | positive for the additional |
| | | | Candida krusei | Candida krusei, | organism |
| | | | | Staphylococcus epidermidis | |
| S110 | BAL | Validation | Aspergillus spp | Pseudomonas aeruginosa, | Two BALs 3 days later |
| | | | (mixed morphotypes present) | Aspergillus spp | positive for Pseudomonas |
| | | | | (mixed morphotypes present) | aeruginosa and was treated |

At the optimal Youden's index (nRPM threshold of 2.6 and 0.54 for Illumina and nanopore sequencing, respectively) derived from the training set ROC curve, the sensitivity and specificity of mNGS testing for bacterial detection based on the validation set using the clinical gold standard were 79.2% (95% confidence interval CI 73.5-85.2%), and 90.6% (95% CI 87.3-93.8%), respectively, for Illumina sequencing, compared to 75.0% (95% CI 65.0-85.7%) and 81.4% (95% CI 74.1-89.3%), respectively, for nanopore sequencing (see FIG. 2C). When using the composite standard, the positive percent agreement (PPA) and negative percent agreement (NPA) were 80.0% (95% CI 74.1-86.3%) and 95.3% (95% CI 92.9-97.6%), respectively, for Illumina sequencing, compared to 81.0% (95% CI 72.4-89.7%) and 93.0% (95% CI 88.5-176 96.7%), respectively, for nanopore sequencing (see FIG. 2C; and FIG. 6A-B). Excluding plasma, the performance of mNGS testing was comparable overall among different body fluid types (see FIG. 2D), with the highest accuracy of detection from CSF. Nanopore sequencing yielded similar normalized read counts to Illumina sequencing (p=0.59) (see FIG. 2E). Stratification based on semi-quantitation of culture colonies revealed significantly lower nRPM values for cultures grown from enrichment broth compared to other higher-titer cultures (rare, few, moderate, numerous colonies) (p=0.006) (see FIG. 5F-G).

Among the 34 negative control samples that were negative by culture and 16S-PCR (see FIG. 5H), only one was a false-positive for a bacterial pathogen above the nRPM detection threshold by mNGS (Propionibacterium acnes). Other reads from background contaminating organisms in negative control samples were observed at levels below detection thresholds. Additional bacteria consisting of human commensal organisms, designated mNGS false positives relative to clinical gold standard testing, were also detected in 44% (7 of 16) and 20% (2 of 10) positive control samples by Illumina and nanopore sequencing, respectively. The proportion of reads from each of these presumptive false-positive cases was <5% of the total number of microbial reads in the sample.

Among false-negative cases from both the training and validation sets using the composite threshold, the most common missed organism was Staphylococcus aureus (see Table 1). Illumina sequencing missed 10 of 40 (25%) cases of Staphylococcus aureus, but this was not statistically significant compared to missed cases of infection from other bacteria (12 of 81, 18%) (p=0.21, Fisher's Exact Test). Nanopore sequencing missed 10 of 26 (38.5%) cases of Staphylococcus aureus, statistically significant compared to missed cases from other bacteria (4 of 50, 8%) (p=0.0034, Fisher's Exact Test) (see Table 9).

TABLE 9
Bacterial false positives and false negatives using Illumina and nanopore sequencing

| | | | | | | | | | |
|---|---|---|---|---|---|---|---|---|---|
| | | Training or | Pathogen | | Normalized | % of all | | NGS | Threshhold |
| Case | Sample Type | Validation | Species | RPM | RPM | Microbes | # Reads | call | (nRPM) |

Illumina sequencing - using the Composite Standard

| | | | | | | | | | |
|---|---|---|---|---|---|---|---|---|---|
| S13 | Joint Fluid | Training | Staphylococcus aureus | 2.556 | 1.278040202 | 1 | 2 | FN | 2.60 |
| S24 | Fleural Fluid | Training | Staphylococcus aureus | 0 | 0 | 0 | 0 | FN | 2.60 |
| S39 | Joint Fluid | Training | Staphylococcus aureus | 0.227 | 0.321 | 0.045 | 2 | FN | 2.60 |
| S48 | Abscess | Training | Staphylococcus aureus | 0 | 0 | 0 | 0 | FN | 2.60 |
| S50 | Joint Fluid | Training | Escherichia coli | 14.714 | 0.513 | 0.143 | 76 | FN | 2.60 |
| S51 | BAL | Training | Streptococcus [symbol] | 0.792 | [symbol] | 0.0049 | [symbol] | FP | 2.60 |
| S57 | Peritoneal | Training | Enterococcus [symbol] | 3.147 | [symbol] | [symbol] | 14 | FN | 2.60 |
| | Fluid | | | | | | | | |
| S5 | Joint Fluid | Validation | Staphylococcus aureus | 1.512 | 0.033 | 0.000 | 2 | FN | 2.60 |
| S8 | BAL | Validation | Pseudomonas aeruginosa | 2.217 | 141.860 | 0.167 | 1 | FN | 2.60 |
| S8 | BAL | Validation | Staphylococcus aureus | 0 | 0 | 0 | 0 | FN | 2.60 |
| S12 | Joint Fluid | Validation | Staphylococcus aureus | 0.281 | 0.050 | 0.095 | 2 | FN | 2.60 |
| S23 | Abscess | Validation | Escherichia coli | 0 | 0 | 0 | 0 | FN | 2.60 |
| S53 | Abscess | Validation | Salmonella enterica | 2.871 | 2.846 | 0.027 | 23 | FP | 2.60 |
| S55 | Joint Fluid | Validation | Staphylococcus aureus | 3.994 | 0.999 | 0.491 | [symbol] | FN | 2.60 |
| S56 | Peritoneal | Validation | Enterococcus faecium | 3.215 | 9.094 | 0.002 | 26 | FP | 2.60 |
| | Fluid | | | | | | | | |
| S63 | Joint Fluid | Validation | Staphylococcus [symbol] | 9.571 | 0.423 | 0.776 | [symbol] | FN | 2.60 |
| S11 | Peritoneal | Validation | Staphylococcus aureus | 2.30 | 2.30 | 0.31 | 4 | FN | 2.60 |
| S96 | Lymphocele | Validation | Enterococcus faecalis | 3.28 | 4.64 | 0.00 | 20 | FP | 2.60 |
| S140 | [symbol] | Validation | Staphylococcus [symbol] | 3.25 | 103.96 | 0.11 | 2 | FN | 2.60 |
| S114 | Pleural Fluid | Validation | Methylobacterium | 18.44 | 26.08 | [symbol] | 191 | FP | 2.60 |
| S93 | Peritoneal | Validation | Nocardia farcinica | 0.00 | 0.00 | 0 | 0 | FN | 2.60 |
| S87 | Abscess | Validation | Bartonella [symbol] | 0.85 | 2.40 | 0.13 | 6 | FN | 2.60 |
| S151 | Peritoneal | Validation | Mycobacterium [symbol] | 0 | 0 | 0 | 0 | FN | 2.60 |
| S144 | Fluid [symbol] | Validation | Mycobacterium avium | 0 | 0 | 0 | 0 | FN | 2.60 |
| S152 | Peritoneal | Validation | Mycobacterium [symbol] | 0 | 0 | 0 | 0 | FN | 2.60 |
| S122 | BAL | Validation | Staphylococcus [symbol] | [symbol] | 108.88 | 0.14 | 1083 | FP | 2.60 |
| S145 | Pleural Fluid | Validation | Nocardia nova | 0.18 | 0.13 | 0.00 | 1 | FN | 2.60 |
| S143 | FNA [symbol] | Validation | Mycobacterium avium | 0 | 0 | 0 | 0 | FN | 2.60 |
| S167 | Pleural Fluid | Validation | [symbol] | [symbol] | 6.45 | 0.77 | 598 | FP | 2.60 |
| S86 | Fluid left | Validation | Staphylococcus aureus | 75.026 | 1.65785554 | 0.2728558 | 579 | FN | 2.60 |
| | heel (swab) | | | | | | | | |

Nanopore Sequencing - using the Composite Standard

| | | | | | | | | | |
|---|---|---|---|---|---|---|---|---|---|
| S24 | Pleural Fluid | Training | Staphylococcus aureus | 0 | 0 | 0 | 0 | FN | 0.5368 |
| S38 | Abscess | Training | Staphylococcus aureus | 2.01998 | 8.079910313 | 1 | 2 | FN | 0.5368 |
| S39 | Joint Fluid | Training | Staphylococcus aureus | 0 | 0 | 0 | 0 | FN | 0.5368 |
| S48 | Abscess | Training | Staphylococcus aureus | 0 | 0 | 0 | 0 | FN | 0.5368 |
| S40 | Pleural Fluid | Training | [symbol] | 60.498 | 0.945281013 | 0.43448276 | 63 | FP | 0.5368 |
| S50 | Joint Fluid | Training | Escherichia coli | 12.365 | 0.430337206 | 0.17391304 | 12 | FN | 0.5368 |
| S5 | Joint Fluid | Validation | Staphylococcus aureus | 0 | 0 | 0 | 0 | FN | 0.5368 |
| S8 | BAL | Validation | Staphylococcus aureus | 0 | 0 | 0 | 0 | FN | 0.5368 |
| S12 | Joint Fluid | Validation | Staphylococcus aureus | 0 | 0 | 0 | 0 | FN | 0.5368 |
| S23 | Abscess | Validation | Staphylococcus aureus | 0 | 0 | 0 | 0 | FN | 0.5368 |
| S23 | Abscess | Validation | Escherichia coli | 0 | 0 | 0 | 0 | FN | 0.5368 |
| S36 | Abscess | Validation | Staphylococcus aureus | 1.15669 | 4.62674327 | 1 | 1 | FN | 0.5368 |
| S41 | Pleural Fluid | Validation | Enterococcus faecium | 2.42234 | 4.844673705 | 0.05 | 4 | FP | 0.5368 |
| S56 | Peritoneal Fluid | Validation | Enterococcus faecium | 2.95493 | 8.357799853 | 0.003663 | 3 | FP | 0.5368 |
| S59 | Peritoneal Fluid | Validation | Neisseria [symbol] | 4.01521 | 1.419592873 | 0.00082781 | 6 | FP | 0.5368 |
| S59 | Peritoneal Fluid | Validation | [symbol] | 2.67681 | 0.946395249 | 0.00055188 | 4 | extra FP | 0.5368 |
| S63 | Joint Fluid | Validation | Staphylococcus [symbol] | 9.4256 | [symbol] | 1 | 10 | FN | 0.5368 |
| S55 | Joint Fluid | Validation | Staphylococcus aureus | 1.80027 | 0.450067447 | 0.833333333 | 10 | FN | 0.5368 |
| S31 | Pleural Fluid | Validation | Klebsiella pneumoniae | 144.752 | 12.79439235 | 0.84023669 | 142 | FP | 0.5368 |
| S31 | Pleural Fluid | Validation | [symbol] | 0 | 0 | 0 | 0 | FN | 0.5368 |

Illumina sequencing using the Gold Standard

| | | | | | | | | | |
|---|---|---|---|---|---|---|---|---|---|
| S13 | Joint Fluid | Training | Staphylococcus aureus | [symbol] | [symbol] | 1 | [symbol] | FN | 2.60 |
| S24 | Pleural Fluid | Training | Staphylococcus aureus | 0 | 0 | 0 | 0 | FN | 2.60 |
| S39 | Joint Fluid | Training | Staphylococcus aureus | [symbol] | 0.321 | 0.043 | [symbol] | FN | 2.60 |
| S48 | Abscess | Training | Staphylococcus aureus | 0 | 0 | 0 | 0 | FN | 2.60 |
| S50 | Joint Fluid | Training | Escherichia coli | 14.714 | 0.513 | 0.143 | 76 | FN | 2.60 |
| S50 | Joint Fluid | Training | Klebsiella pneumoniae | [symbol] | [symbol] | 0.60338346 | 321 | FP | 2.60 |
| S51 | BAL | Training | Streptococcus pneumoniae | 0.792 | 3.168 | 0.049 | 6 | FP | 2.60 |
| S57 | Peritoneal Fluid | Training | Enterococcus faecium | 3.147 | 0.278 | 0.030 | 14 | FN | 2.60 |
| S57 | Peritoneal Fluid | Training | Klebsiella pneumoniae | 39.5573 | [symbol] | [symbol] | [symbol] | FP | 2.60 |
| S5 | Joint Fluid | Validation | Staphylococcus aureus | 1.512 | 0.033 | 0.000 | [symbol] | FN | 2.60 |
| S8 | BAL | Validation | Staphylococcus aureus | 0 | 0 | 0 | 0 | FN | 2.60 |
| S12 | Joint Fluid | Validation | Staphylococcus aureus | 0.281 | 0.050 | 0.095 | 2 | FN | 2.60 |
| S23 | Abscess | Validation | Escherichia coli | 0 | 0 | 0 | 0 | FN | 2.60 |
| S53 | Abscess | Validation | Salmonella enterica | 2.871 | 2.846 | 0.027 | 23 | FP | 2.60 |
| S55 | Joint Fluid | Validation | Staphylococcus aureus | 3.994 | 0.999 | 0.491 | 26 | FN | 2.60 |
| S56 | Peritoneal Fluid | Validation | Enterococcus faecium | 3.215 | 9.094 | 0.002 | 26 | FP | 2.60 |
| S63 | Joint Fluid | Validation | Staphylococcus [symbol] | 9.571 | 0.423 | 0.776 | 66 | FN | 2.60 |
| S11 | Peritoneal | Validation | Staphylococcus aureus | 2.30 | 2.30 | 0.31 | 4 | FN | 2.60 |
| S96 | [symbol] | Validation | Enterococcus faecalis | 3.28 | 4.64 | 0.00 | 20 | FP | 2.60 |
| S140 | Seroma [symbol] | Validation | Staphylococcus [symbol] | 3.25 | 103.96 | 0.11 | 2 | FN | 2.60 |
| S114 | Pleural Fluid | Validation | Methylobacterium | 18.44 | 26.08 | 0.07 | 191 | FP | 2.60 |
| S93 | Peritoneal | Validation | Nocardia [symbol] | 0.00 | 0.00 | 0 | 0 | FN | 2.60 |
| S151 | Peritoneal | Validation | Mycobacterium tuberculosis | 0 | 0 | 0 | 0 | FN | 2.60 |
| S144 | Fluid right [symbol] | Validation | Mycobacterium avium | 0 | 0 | 0 | 0 | FN | 2.60 |
| S152 | Peritoneal | Validation | Mycobacterium tuberculosis | 0 | 0 | 0 | 0 | FN | 2.60 |
| S122 | BAL | Validation | Staphylococcus [symbol] | 76.99 | 108.88 | 0.14 | 108.3 | FP | 2.60 |
| S145 | Pleural Fluid | Validation | Nocardia nova | 0.18 | 0.13 | 0.00 | 1 | FN | 2.60 |
| S145 | FNA right | Validation | Mycobacterium avium | 0 | 0 | 0 | 0 | FN | 2.60 |
| S167 | Pleural Fluid | Validation | [symbol] | 51.57 | 6.45 | 0.77 | 598 | FP | 2.60 |
| S86 | Fluid left | Validation | Staphylococcus aureus | 75.026 | 1.65785554 | 0.2728558 | 579 | FN | 2.60 |
| | heel (swab) | | | | | | | | |
| S17 | Pleural Fluid | Validation | Enterococcus faecium | 12.2439 | 48.97550888 | 0.0332259 | 80 | FP | 2.60 |
| S19 | Pleural Fluid | Validation | Enterococcus faecium | 12.3793 | 4.376744127 | 0.06299213 | 8 | FP | 2.60 |
| S31 | Pleural Fluid | Validation | Klebsiella pneumoniae | 204.842 | 18.1056719 | 0.81829733 | 644 | FP | 2.60 |

[symbol]

indicates data missing or illegible when filed

For fungal pathogen detection, a clinical gold standard consisting of available culture and 28S-ITS PCR results was used. On average, fungal DNA was at a significantly lower concentration based on nRPM counts than bacterial DNA (p=0.0049) (see FIG. 2F). At the optimal Youden's index derived from the training set ROC curve (nRPM=0.1 for both Illumina and nanopore sequencing), the sensitivity and specificity of mNGS detection of fungi using an independent validation set were 90.6% (95% CI 84.2-100%) and 89.0% (95% CI 85.7-92.5%), respectively, for Illumina sequencing (n=127 samples), compared to 90.9% (95% CI 80.0-100%) and 100%, respectively, for nanopore sequencing (n=43 samples) (see FIG. 2C; FIG. 6C-D; and Table 4). Among the false-negative cases in the training and validation sets, at least 1 read corresponding to the fungal pathogen was detected in 57% (4 of 7) and 17% (1 of 6) samples by Illumina and nanopore sequencing, respectively, suggesting that sensitivity could potentially be boosted at greater depths of sequencing. The majority of fungal organisms (11 of 14, 79%) designated false-positives by Illumina sequencing were found in <5% of all sequenced microbial reads in the sample.

Limits of Detection (LoD) and Linearity. DNA was spiked from a mixture of 4 organisms that were non-pathogenic to humans (Streptococcus uberis, Rhodobacter sphaeroides, Millerozyma farinosa, and Aspergillus oryzae) into healthy donor plasma matrix for LoD evaluation. Samples were spiked in 4-fold dilutions, ranging from 1:1 (no dilution) to 1:4096 dilution, with 4 replicates at each dilution. The LoD for bacterial detection using this assay was estimated to be between 400-700 genome equivalents (GE) per mL for bacteria and 4 GE per mL for fungi (see Table 10). A strong linear correlation between the organism titer (GE/mL) and nRPM values by mNGS was observed (R2=0.89-0.98; see FIG. 7).

TABLE 10
LoD expressed in genome equivalents per mL (GE/mL)

| | | mNGS Result (Positive/Negative, # of Positive Results, | | | | | | |
|---|---|---|---|---|---|---|---|---|
| Organism | Genome | Organism Copies in GE/mL) | | | | | | |
| Copies | Size | Dilution Factor | | | | | | |
| (GE/mL) | (Mb) | 1 | 4 | 16 | 64 | 256 | 1024 | 4096 |
| Streptococcus | 1.9 | POSTIVE | POSITIVE | POSITIVE | POSITIVE | NEGATIVE | NEGATIVE | NEGATIVE |
| uberis | | (4 of 4) | (4 of 4) | (4 of 4) | (4 of 4) | (0 of 4) | (0 of 4) | (0 of 4) |
| | | 27,300 | 6,830 | 1,710 | 427 | 107 | 26.7 | 6.7 |
| | | GE/mL | GE/mL | GE/mL | GE/mL | GE/mL | GE/mL | GE/mL |
| Rhodobacter | 4.6 | POSITIVE | POSITIVE | POSITIVE | NEGATIVE | NEGATIVE | NEGATIVE | NEGATIVE |
| sphaeroides | | (4 of 4) | (4 of 4) | (4 of 4) | (0 of 4) | (0 of 4) | (0 of 4) | (0 of 4) |
| | | 11,000 | 2,750 | 689 | 172 | 43 | 10.8 | 2.7 |
| | | GE/mL | GE/mL | GE/mL | GE/mL | GE/mL | GE/mL | GE/mL |
| [symbol] | 14 | POSITIVE | POSITIVE | POSITIVE | POSITIVE | POSITIVE | NEGATIVE | NEGATIVE |
| | | (4 of 4) | (4 of 4) | (4 of 4) | (4 of 4) | (4 of 4) | (0 of 4) | (0 of 4) |
| | | 4,800 | 1,120 | 280 | 70 | 17.4 | 4.4 | 1.1 |
| | | GE/mL | GE/mL | GE/mL | GE/mL | GE/mL | GE/mL | GE/mL |
| Aspergillus | 37 | POSTIVE | POSTIVE | POSTIVE | POSTIVE | NEGATIVE | NEGATIVE | NEGATIVE |
| oryzae | | (4 of 4) | (4 of 4) | (4 of 4) | (4 of 4) | (0 of 4) | (0 of 4) | (0 of 4) |
| | | 1,100 | 276 | 280 | 17.4 | 4.3 | 1.1 | 0.3 |
| | | GE/mL | GE/mL | GE/mL | GE/mL | GE/mL | GE/mL | GE/mL |

Abbreviations: GE, genome equivalents; LoD, limit of detection; Mb, megabases. [symbol]
indicates data missing or illegible when filed

Case Series. To assess the potential clinical utility of body fluid mNGS for diagnosis of infection, 12 patients were selectively enrolled with clinically probable or established infection despite negative culture and/or PCR testing of the body fluid (see Table 11). An infectious diagnosis had been made by direct detection from a different body fluid/tissue or by serology/chemistry in 8 and 3 cases respectively. A peritoneal fluid from a patient with bowel perforation and suspected abdominal infection was also included. Presumptive causative pathogens (Klebsiella aerogenes, Aspergillus fumigatus, Streptococcus pneumoniae, Streptococcus pyogenes, Cladophialophora psammophila, Candida parapsilosis, and anaerobic gastrointestinal microbiota) were identified in 7 of 12 cases using mNGS (see Table 11; and Clinical Vignettes in the Examples). Two additional cases of Treponema pallidum (neurosyphilis) and Coccidioides immitis (coccidioidomycosis), diagnosed by serology, had reads detected but present at levels below pre-established nRPM reporting thresholds. Among the remaining 3 cases, mNGS testing was unable to detect Cryptococcus neoformans in pleural fluid (diagnosis made from a culture-positive BAL fluid), Mycobacterium tuberculosis in pleural fluid (diagnosis made from a positive lymph node culture), and Sporothrix sp. in CSF (diagnosis made from serum and CSF IgM antibody positivity), presumably due to a lack of DNA representation from absent or very low pathogen titers and/or high human host background in the body fluid.

TABLE 11
Case series of body fluid mNGS testing in patients with probable or established infection but negative clinical microbiological testing.

| | | | | Body Fluid | |
|---|---|---|---|---|---|
| | Body Fluid | | Body Fluid | 16S/28S-ITS | Clinical Microbiological |
| Case | Sample Type | Presentation | mNGS Result | PCR Results^{a} | Diagnosis |
| S88 | CSF | Encephalopathy without | Positive | Negative | Klebsiella aerogenes: |
| | | known cause; has a brain | (Klebsiella aerogenes) | | Same organism grown in |
| | | implant | | | culture from surgically |
| | | | | | removed deep brain |
| | | | | | stimulator. Concordant |
| | | | | | dPCR. Matches clinical |
| | | | | | context. |
| S89 | Retro- | Abdominal fluid | Positive (multiple | ND | None: mNGS results |
| | uterine | collection and elevated | GI anaerobes)^{b} | | consistent with clinical |
| | fluid | white blood cell count; | | | context |
| | | a history of abdominal | | | |
| | | surgery | | | |
| S90 | Pleural | Fever, cough, bacteremia, | Positive | ND | Streptococcus pneumoniae: |
| | | loculated pleural effusion | (Streptococcus pneumoniae) | | Concordant positive blood |
| | | | | | culture matches the |
| | | | | | clinical context |
| S91 | Pleural | Fever, bacteremia, | Positive | ND | Streptococcus pyogenes: |
| | | pneumonia, pleural | (Streptococcus pyogenes) | | Concordant positive |
| | | effusion | | | blood culture matches the |
| | | | | | clinical context |
| S92 | BAL | Pulmonary nodules | Positive | ND | Aspergillus fumigatus: |
| | | post-chemotherapy | (Aspergillus fumigatus) | | Probable invasive |
| | | | | | aspergillosis matches |
| | | | | | clinical context. Serum |
| | | | | | beta-D-glucan and serum |
| | | | | | galactomannan positive. |
| S176 | BAL | Cavitary lesion | Positive - below threshold | ND | Coccidioides immitis: |
| | | of the lung | (Coccidioides immitis) | | Serum coccidioides |
| | | | | | antibody positive, 1:16 |
| | | | | | titer on complement |
| | | | | | fixation. |
| S177 | CSF | Chronic | Positive - | Negative | Cladophialophora bantiana: |
| | | meningoencephalitis | (Cladophialophora bantiana) | | Brain tissue culture: rare |
| | | | | | Cladophialophora bantiana |
| S178 | Pleural | Cavitary lung lesions | Negative^{c} | ND | Cryptococcus neoformans: |
| | Fluid | and pleural effusion | | | BAL and bronchial wash fluid |
| | | | | | culture positive for |
| | | | | | Cryptococcus neoformans. |
| | | | | | Serum CrAg positive. |
| S179 | CSF | Headache, vision changes, | Positive - | Negative | Treponema pallidum: |
| | | and optic disc edema | Treponema pallidum | | serum RPR, VDRL, and treponemal |
| | | | (below threshold) | | antibody positive |
| S180 | Pleural | Lymphadenopathy, | Negative^{c} | ND | Mycobacterium tuberculosis: |
| | Fluid | lymphocytic pleural | | | Positive PPD. Lymph node |
| | | effusion | | | biopsy: Necrotizing granulomas, |
| | | | | | AFB stain positive, and MTB |
| | | | | | PCR positive. |
| S181 | CSF | Headaches, blurred vision, | Candida parasilopsis | Negative | Candida parasilopsis: |
| | | night sweats, neck stiffness | | | Serum beta-D-glucan: >500. CSF |
| | | | | | culture 24 days later positive for |
| | | | | | Candida parasilopsis. |
| S182 | CSF | Headache, photophobia, | Negative^{c} | Negative | Sporothrix schenkii: |
| | | blurry vision, hydrocephalus. | | | CSF (>1:16) and serum (1:32) |
| | | | | | Sporothrix antibody positive. |

^{a}Bacterial 16S PCR or fungal 28S-ITS PCR
^{b}The top 5 anaerobes were Faecalibacterium prausnitzii, Eubacterium rectale, Akkermansia muciniphila, Acidaminococcus intestini, and Bifidobacterium adolescentis.
^{c}Infectious diagnosis missed by mNGS testing
Abbreviations: dPCR: digital PCR; ND, not done; MTB, Mycobacterium tuberculosis

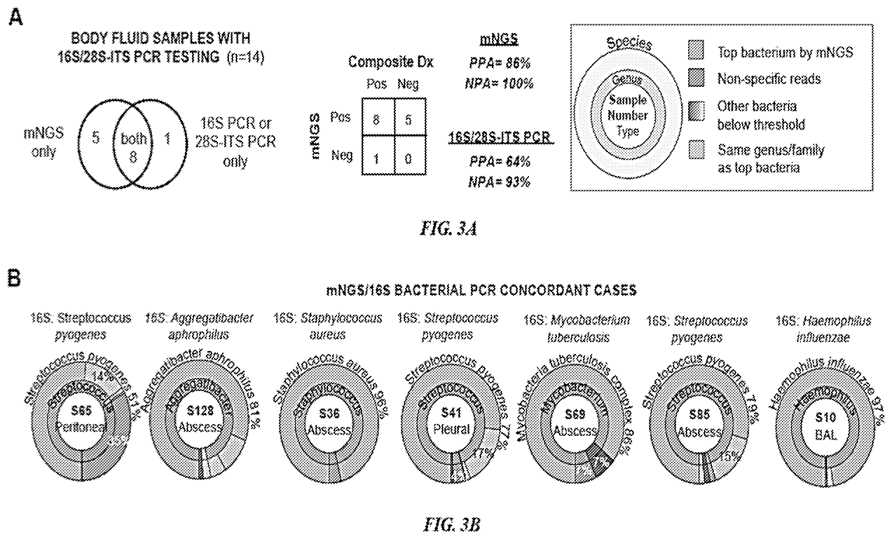

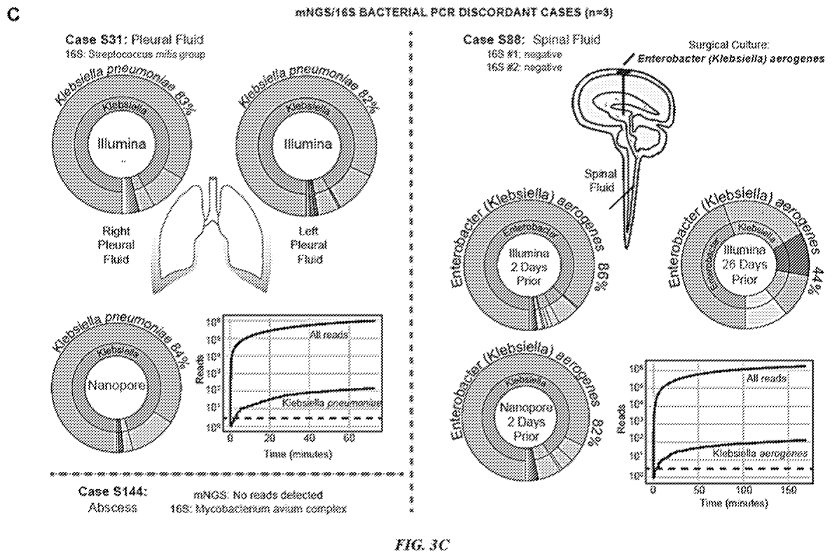

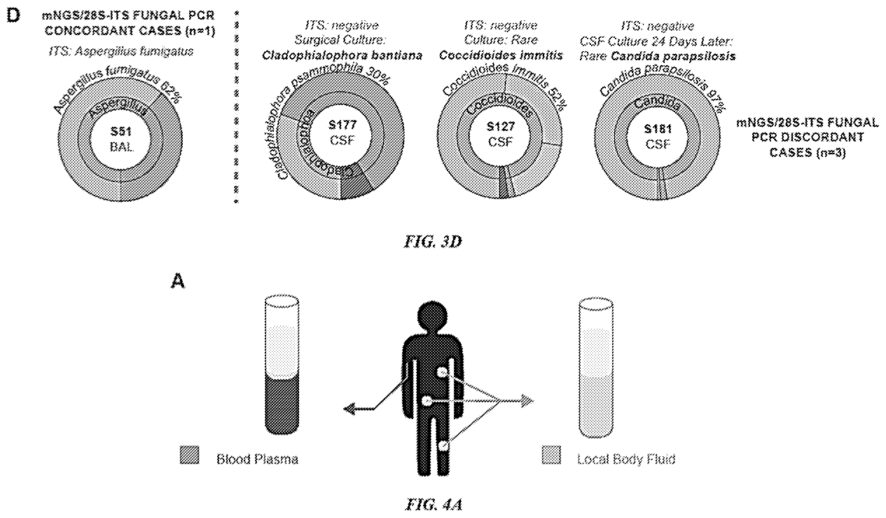

Comparison of mNGS with bacterial 16S and fungal 28S-ITS PCR. Out of the 160 patients, the performance of mNGS relative to bacterial 16S PCR or fungal 28S-ITS PCR was compared in 14 cases that had 16S or 28S-ITS PCR testing performed out of 160 (see FIG. 3, and Table 12). At the hospital, bacterial 16S and fungal 28S-248 ITS PCR testing of body fluids and tissue are routinely ordered for culture-negative cases with high clinical suspicion for infection. Concordant results between mNGS testing and PCR were obtained in 8 of 14 cases (see FIGS. 3A-B and D). Of the 6 discordant cases, 5 were found only by mNGS and 1 only by 16S PCR.

TABLE 12
Comparison of bacterial 16S and fungal 28S-ITS PCR and mNGS results.

| | | | | Body Fluid - | | |
|---|---|---|---|---|---|---|
| | Sample | | Pathogen | Normalized | PCR | Orthogonal |
| Case #^{a} | Type | Syndrome | Species | RPM^{b} | result | testing |
| S10 | BAL | Respiratory infection - | Haemophilus | 22,192 | Haemophilus | No further |
| | fluid | pneumonia and | influenzae | | influenzae | testing |
| | | respiratory failure | | | (16S PCR) | |
| S31 | Pleural | Respiratory infection - | Klebsiella | 18.12 | Streptococcus mitis | Digital PCR, |
| | Fluid | necrotizing pneumonia | pneumoniae | | group (16S PCR) | Sanger |
| | | | | | | sequencing |
| | | | | | | contralateral |
| | | | | | | pleural fluid |
| | | | | | | [symbol] |
| S36 | Abscess | Abscess of Extremity | Staphylococcus | 4.38 | Staphylococcus aureus | No further |
| | | | aureus | | (16S PCR) | testing |
| S41 | Pleural | Respiratory infection | Streptococcus | 132.4 | Streptococcus | No further |
| | Fluid | | pyogenes | | pyogenes (16S PCR) | testing |
| S65 | Peritoneal | Peritonitis | Streptococcus | 6.4 | Streptococcus | No further |
| | fluid | | pyogenes | | pyogenes (16S PCR) | testing |
| S69 | Abscess | Gastrointestinal | Mycobacterium | 6.79 | Mycobacterium | No further |
| | | abscess | tuberculosis | | tuberculosis (16S PCR) | testing |
| S85 | Abscess | CNS abscess | Streptococcus | 3.2 | Streptococcus | No further |
| | | | pyogenes | | pyogenes (16S PCR) | testing |
| S88 | CSF | CNS infection - | Klebsiella | 79 | Not detected | Culture of |
| | | hardware infection | (Enterobacter) | | | surgically |
| | | | aerogenes | | | removed CNS |
| | | | | | | implant; digital |
| | | | | | | PCR |
| S51 | BAL fluid | Respiratory infection | Aspergillus | 3.17 | Aspergillus | No further |
| | | | fumigatus | | fumigatus | testing |
| | | | | | (23S-ITS PCR) | |
| S127 | CSF | Lymphocytic | Coccidioides | 15.6 | Not detected | Culture-positive |
| | | meningitis | [symbol] | | | |
| S177 | CSF | [symbol] | [symbol] | 0.03 | Not detected | Culture of |
| | | and hydrocephalus | | | | surgical brain |
| | | | | | | biopsy matches |
| | | | | | | NGS |
| S181 | CSF | Chronic meningitis | Candida | 11.82 | Not detected | Culture positive |
| | | and intracranial | [symbol] | | | in CSF 24 days |
| | | hypertension | | | | later |

^{a}all samples in this table had negative testing results by culture, but the pathogen was detected by 16S PCR, mNGS, and/or orthogonal testing, and all were clinically adjudicated if there was a discrepancy between tests.
^{b}does not take into account any dilution of DNA extract prior to library preparation.
^{c}Klebsiella pneumoniae pathogen was detected by mNGS, but not by 16S PCR. To resolve the discrepancy, this sample was also confirmed positive for the pathogen by digital PCR (dPCR) and Sanger sequencing from the original pleural fluid and the contralateral pleural fluid. See FIG. 3B and Clinical Vignettes in the Examples for further details.
Abbreviations: BAL, bronchoalveolar lavage. [symbol]
indicates data missing or illegible when filed

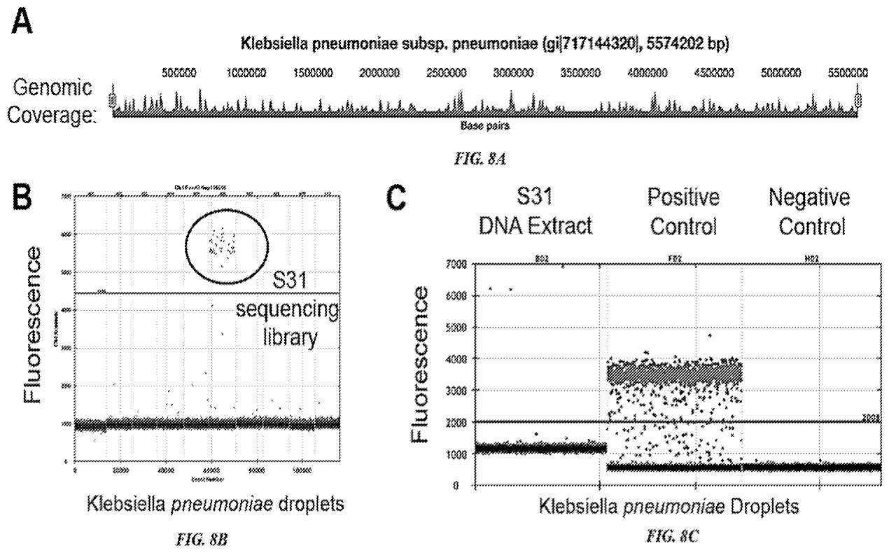

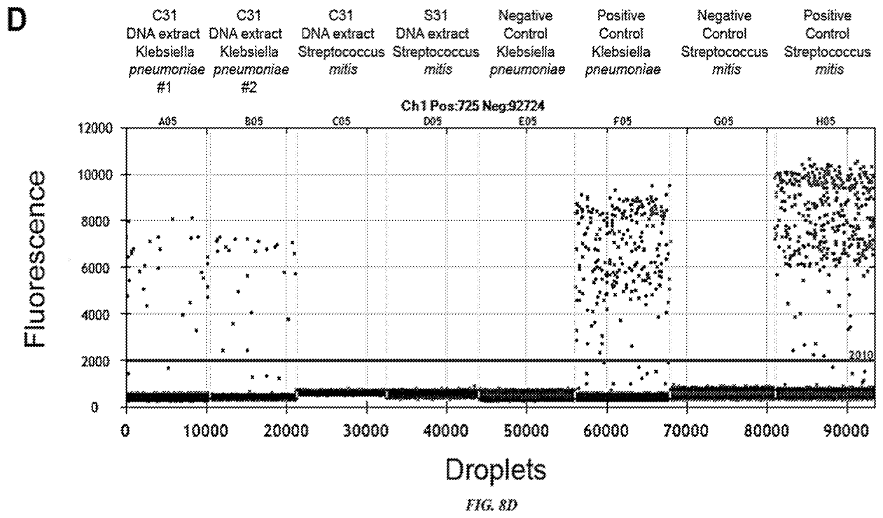

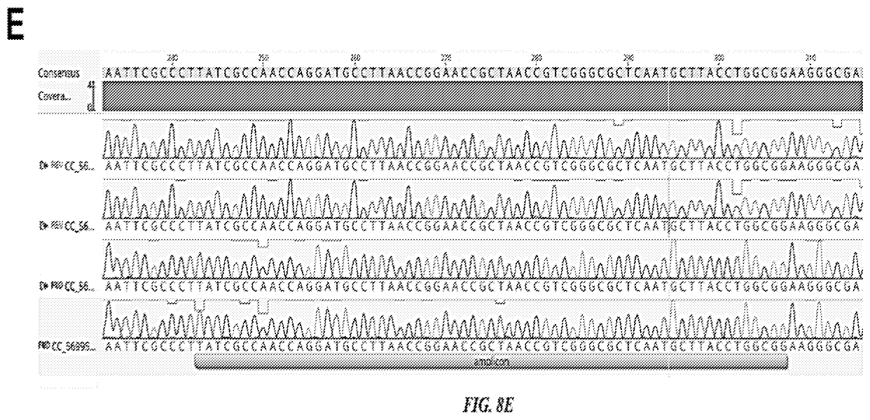

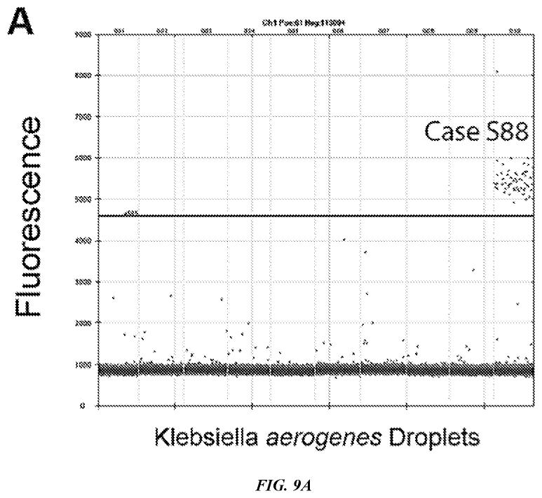

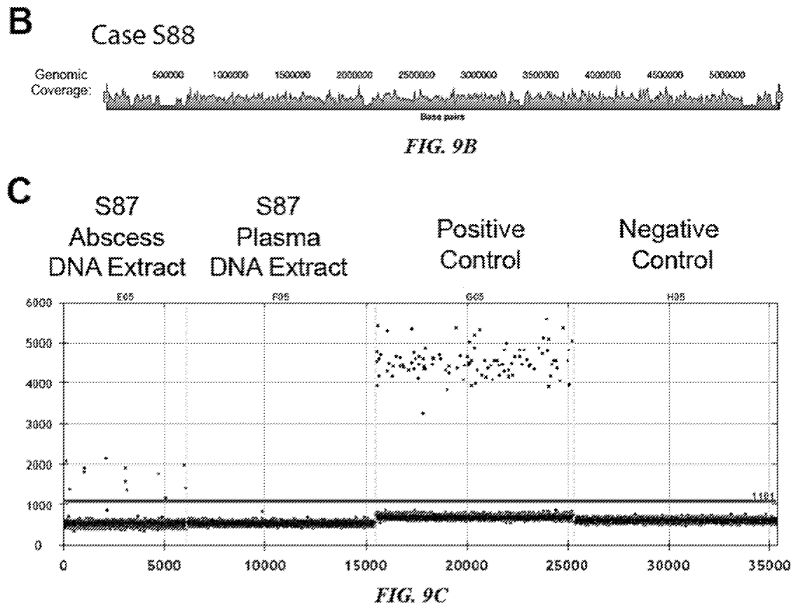

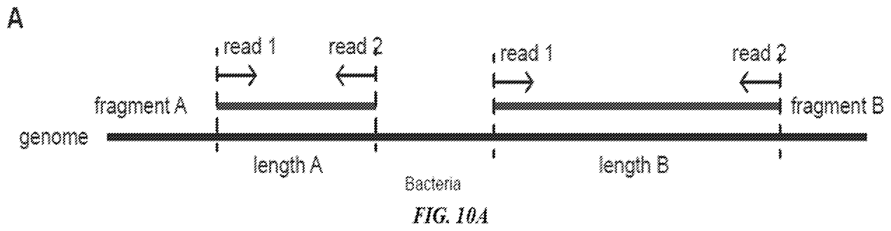

The first of 3 discordant bacterial cases was a case of an immunocompromised child with necrotizing pneumonia (see case S31 in Table 12, see FIG. 3C, see Clinical Vignettes in the Examples). 16S PCR testing of the pleural fluid was positive for an organism in the Streptococcus mitis group, whereas mNGS testing identified Klebsiella pneumoniae (see FIG. 3C). The finding of Klebsiella pneumoniae by mNGS was orthogonally validated as correct using 5 approaches: (i) dPCR of the DNA extract, (ii) dPCR of the sequencing library, (iii) Sanger sequencing of PCR amplicons from the DNA extract, (iv) mNGS (Illumina) sequencing of the contralateral pleural fluid showing Klebsiella pneumoniae, and (v) dPCR of the contralateral pleural fluid (see FIG. 8). In the second case, although culture and 16S PCR testing of the body fluid (CSF) were both negative, a subsequent culture from a neurosurgically removed deep brain stimulator (DBS) was positive for Klebsiella aerogenes (see case S88 of Table 12, see FIG. 3C, and Clinical Vignettes in the Examples). mNGS testing of CSF was also positive for Klebsiella aerogenes, a finding that was orthogonally validated with dPCR (see FIG. 9). The length distribution was analyzed of the pathogens detected by mNGS for these two cases using paired-end sequencing. The mean lengths of species-specific pathogen reads were 77 and 71 nucleotides (nt), with nearly all lengths <300 nt. The third discordant case was an abscess fluid that was positive by 16S PCR for Mycobacterium avium complex but negative by mNGS testing.

In all 3 discordant fungal cases, body fluid mNGS was able to find the causative organism, whereas fungal 28S-ITS PCR testing was negative (see FIG. 3D). The causative organism had been clinically confirmed by culture of the same body fluid (n=1), culture done 24 days later (n=1), or testing of brain biopsy tissue (n=1).

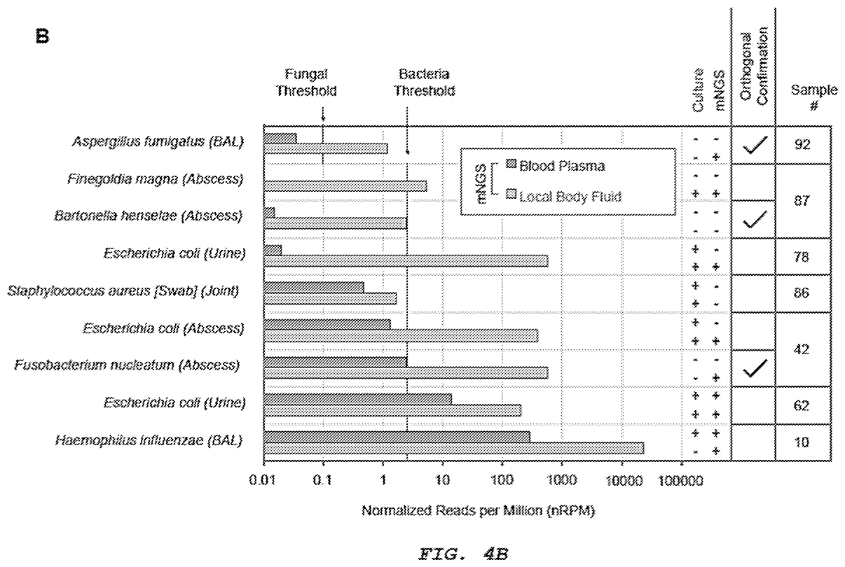

Comparison of diagnostic yield of mNGS testing from body fluids versus plasma. Seven patients in the study harboring a total of 9 pathogens had paired body fluid and plasma samples available for comparative mNGS testing (See Table 13). Pathogen cfDNA burden based on nRPM was a median 160-fold higher (IQR 34-298) in the local body fluid than in plasma from the same patient (see FIG. 4 and Table 13).

TABLE 13
Comparison of results from mNGS of paired plasma and body fluids.

| | | | | | | Body | |
|---|---|---|---|---|---|---|---|
| | | | | Body | Blood | Fluid: | |
| | | | | Fluid - | Plasma - | Plasma | Time |
| | Sample | | Pathogen | Normalized | Normalized | Fold | Delta^{b} |
| Case # | Type | Syndrome | Species | RPM^{a} | RPM^{a} | Difference | (Days) |
| S10 | Bronchial | Respiratory infection - | Haemophilus | 22.192 | 276 | 60.41 | −1.21 |
| | Lavage | pneumonia and | influenzae | | | | |
| | | respiratory failure | | | | | |
| S92 | Bronchial | Respiratory infection - | Asperigillus | 1.19 | 0.035 | [symbol] | 4.51 |
| | Lavage | pulmonary modules | fumigatus | | | | |
| S42 | Abscess | Gastrointestinal | Fusobacterium | 544 | 2.45 | 222.04 | 5.23 |
| | | Abscess | [symbol] | | | | |
| S42 | Abscess | Gastrointestinal | Escherichia coli | 387.2 | 1.3 | 297.65 | [symbol] |
| | | Abscess | | | | | |
| S62 | Urine | Urinary Tract infection | Escherichia coli | 204.27 | 14.14 | 14.44 | −0.19 |
| S76 | Urine | Urinary Tract infection | Escherichia coli | [symbol] | 0.019 | 28755 | −0.44 |
| S55 | Joint (swab) | Septic Joint | Staphylococcus | 1.66 | 0.46 | 3.6 | 0.64 |
| | | | aureus | | | | |
| S87 | Abscess | Axillary Abscess | [symbol] | 2.4 | 0.015 | 160 | 1.42 |
| S87 | Abscess | Axillary Abscess | [symbol] | 5.36 | 0 | infinite | 1.42 |

^{a}does not take into account any dilution of DNA extract prior to library preparation.
^{b}difference in days between plasma and body fluid collection; for example, 4.51 refers to plasma collection done 4.51 days before the body fluid collection
Abbreviations: RPM, reads per million. [symbol]
indicates data missing or illegible when filed

Detection of Anaerobic Bacteria and Viruses. Anaerobic bacteria were not included in the accuracy assessment, as anaerobic culture was not always performed and cultured anaerobes were typically not speciated. However, the one sample in the accuracy study that was culture-positive for an anaerobic bacterium (Finegoldia magna from a soft tissue abscess, Case S87 of Table 13) was successfully detected by mNGS testing (See Table 14).

TABLE 14
Anaerobic bacteria detected by mNGS in the accuracy study.

| | | | | % of all | | |
|---|---|---|---|---|---|---|
| | | | Normalized | Microbial | | Sample |
| Case # | Species | RPM | RPM | Reads | Reads | Type |
| S10 | Rothia dentocariosa | 5.28 | 10.57 | 0.00046 | 4 | BAL |
| S37 | Peptoclostriudium difficile | 1.67 | 3.34 | 0.00064 | 9 | Perihepatic Fluid |
| S74 | Peptoclostriudium difficile | 0.51 | 2.86 | 0.0009 | 4 | Peritoneal Fluid |
| S56 | Prevotella denticola | 1.85 | 5.25 | 0.001 | 15 | Peritoneal Fluid |
| S37 | Campylobacter curvus | 2.6 | 5.19 | 0.001 | 14 | Perihepatic fluid |
| S56 | gamma proteobacterium HdN1 | 2.23 | 6.3 | 0.0012 | 16 | Peritoneal Fluid |
| S59 | Veillonella parvula | 7.74 | 2.73 | 0.0012 | 55 | Peritoneal Fluid |
| S56 | Lactococcus lactis | 3.71 | 10.49 | 0.002 | 30 | Peritoneal Fluid |
| S5 | Geobacillus sp. WCH70 [symbol] | 139.67 | 2.71 | 0.0023 | 185 | Joint Fluid |
| S34 | Parvimonas micra | 0.46 | 2.59 | 0.0052 | 3 | Pleural Fluid |
| S5 | Bacillus coagulans | 341.73 | 7.55 | 0.0057 | 452 | Joint Fluid |
| S59 | Campylobacter concisus | 51.05 | 18.05 | 0.008 | 363 | Peritoneal Fluid |
| S74 | Bifidobacterium breve | 16.44 | 92.98 | 0.029 | 130 | Peritoneal Fluid |
| S37 | Lactobacillus gasseri | 77.89 | 155.78 | 0.03 | 420 | Perihepatic fluid |
| S53 | Bacteroides xylanisolvens | 4.24 | 16.97 | 0.041 | 34 | Abscess |
| S59 | Lactococcus lactis | 416.57 | 147.28 | 0.065 | 2962 | Peritoneal Fluid |
| S37 | Prevotella melaninogenica | 686.42 | 1376.83 | 0.26 | 3712 | Perihepatic Fluid |
| S51 | Prevotella melaninogenica | 4.62 | 18.48 | 0.29 | 35 | BAL |
| S56 | Veillonella parvula | 677.79 | 1917.08 | 0.38 | 5481 | Peritoneal Fluid |
| S42 | Fusobacterium nucleatum | 135.97 | 932.59 | 0.44 | 59 | Abscess |
| S87 | Fingoidia magna | 2.68 | 7.59 | 0.41 | 19 | Abscess |
| S116 | Streptococcus [symbol] | 86.55 | 2.70 | 0.25 | 731 | BAL |
| S149 | Peptoclostriudium difficile | 3.73 | 2.64 | 0.00 | 32 | Peritoneal Fluid |
| S176 | Veillonella parvula | 177.50 | 7.84 | 0.16 | 1186 | BAL |
| S176 | Rothia [symbol] | 110.45 | 4.88 | 0.10 | 738 | BAL |
| S137 | Veillonella parvula | 6.83 | 218.54 | 0.01 | 1 | Retrogastric Fluid |

Abbreviations: BAL, bronchoalveolar lavage; RPM, reads per million. [symbol]
indicates data missing or illegible when filed

DNA viruses were also excluded in the accuracy assessment due to lack of routine clinical testing for viruses. Applying previously validated clinical mNGS thresholds of 3 non-overlapping reads for viral detection viruses were detected from the Anelloviridae (n=5), Herpesviridae (n=9), and Adenoviridae (n=2) families (See Table 15). Four of the 5 (80%) anellovirus detections were from immunocompromised patients, consistent with the reported association of anelloviruses as non-pathogenic markers of active inflammation in this population. Among the 11 remaining viruses detected by body fluid mNGS, 6 of 6 (100%) were orthogonally confirmed as true-positive cases by virus-specific PCR.

TABLE 15
Viruses detected by mNGS in the accuracy study.

| | | | | | PCR Testing in | |
|---|---|---|---|---|---|---|
| | | | | | Clinical | |
| | | | | | Microbiology | |
| | | mNGS | | | Laboratory | |
| | Sample | Detected | | Clinical Viral | (Research Use | |
| Sample | Type | Virus | Reads | Testing +/−7 days | Only) | Comments |
| S15 | Bronchial | HHV5 | 6 | Bronchial Lavage CMV | Positive CMV - | CMV infection was |
| | Lavage | | | [symbol] | 1909 IU/mL | diagnosed and |
| | | | | positive, CMV plasma | | treated with |
| | | | | [symbol] Detected: low | | [symbol] |
| | | | | positive, <137 IU/mL | | |
| S22 | Peritoneal | HHV6B | 9 | No testing | No testing | |
| | Fluid | | | | | |
| S27 | Abscess | HHV6B | 193 | No testing | No testing | |
| S37 | Perihepatic | TTV, SENV | [symbol] | No testing | No testing | |
| | Fluid | | | | | |
| S49 | Abdominal | TTV | 433 | No testing | No testing | |
| | fluid (swab) | | | | | |
| S51 | Bronchial | HHV1, | 12, 2 | EBV quant; negative [symbol] | Positive HSV1 | HSV1 infection not diagnosed and |
| | Lavage | low HHV4 | | No testing for Bronchial | (HHV1); Positive | not tested clinically. Patient is a [symbol] |
| | | | | Lavage | EBV (HHV4); 797 | year-old with an autoimmune |
| | | | | | copies/mL | disorder on [symbol] and |
| | | | | | | [symbol] and IgA deficiency who |
| | | | | | | presented with 2-3 weeks of cough, |
| | | | | | | congestion, and fever that |
| | | | | | | progressed to sepsis, bilateral |
| | | | | | | pneumonia, and [symbol] |
| | | | | | | (unclear cause but [symbol] enterovirus) with |
| | | | | | | cardiopulmonary decompensation |
| | | | | | | and [symbol] |
| S57 | Peritoneal | HHV5, | 170, 23, 5 | CMV plasma PCR; | Positive | CMV infection tested 7 days later |
| | Fluid | [symbol] | | Detected [symbol] | Adenovirus [symbol] | and diagnosed upon investigation |
| | | | | | copies/mL | into worsening lung disease of |
| | | | | | | unclear etiology. |
| S58 | Pleural | low HHV5 | 1 | CMV plasma PCR [symbol] | Negative CMV | Patient was seen by an infectious |
| | Fluid | | | Detected [symbol] IU/mL | | disease physician and not [symbol] |
| | | | | | | for CMV in the context of a diagnosis |
| | | | | | | of invasive pulmonary |
| | | | | | | [symbol] |
| S64 | [symbol] | TTV | 13 | No testing | No testing | |
| | (swab) | | | | | |
| S83 | Bronchial | HHV1 | 4 | No testing | No testing; | HSV1 infection not |
| | Lavage | | | | insufficient | diagnosed and |
| | | | | | material | not tested clinically. |
| S85 | Abscess | TTV | 4 | No testing | No testing | |
| S84 | Pleural | low TTV | 2 | No testing | No testing | |
| | Fluid | | | | | |
| S175 | [symbol] | HHV4 | 4 | No testing | No testing | |
| | Fluid | | | | | |
| S176 | Bronchial | [symbol] | 4 | No testing | No testing | |
| | Lavage | | | | | |
| S123 | [symbol] | HHV4 | 21 | EBV [symbol] | No testing | |
| | Fluid | | | days later [symbol] | | |
| | | | | [symbol] copies/mL | | |
| S136 | Bronchial | HHV1 | 3 | CMV culture negative | No testing | |
| | Lavage | | | Blood [symbol] 7 days | | |
| | | | | later was positive [symbol] | | |

[symbol]

indicates data missing or illegible when filed

Clinical Vignettes. The first set of clinical vignettes comprised 5 cases where both culture and 16S PCR was negative, but a clinical diagnosis was made through other means (also shown in Table 11). The cases were sourced from a combination of physician referral and positive microbiological results.

The second set of clinical vignettes is comprised of 7 cases from the accuracy study where mNGS was able to find incidental new bacteria and fungi that were not known at the time of testing. In each case, follow-up orthogonal testing using 16S/ITS PCR or digital PCR was performed and clinical adjudication after mNGS was able to subsequently confirm the new organism.

Set 1: Prospective Case Series of Body Fluid mNGS Testing in Patients with Probable Infection but Negative Clinical Microbiological Testing

Case S88

CSF (2 days prior to surgical removal of the implant):
Culture and 16S rDNA PCR: negative
CSF mNGS Illumina: Klebsiella aerogenes
CSF mNGS Nanopore: Klebsiella aerogenes
dPCR: Klebsiella aerogenes

Deep brain stimulator implant material removed from the brain:
Culture: Klebsiella aerogenes

Clinical adjudication: Klebsiella aerogenes

Case S88 is a man in his 70s with a background of Parkinson's disease, deep brain stimulator (DBS) placement, and mechanical aortic valve replacement on warfarin. The DBS was placed 3 years prior to admission and the electrode was repositioned 9 months prior to admission. The patient was admitted for fever and reduced consciousness with a history of recent traumatic head injury and a scalp wound. He was treated for meningitis with empirical vancomycin, ceftriaxone, and ampicillin, with clinical improvement after six days of treatment. A prompt lumbar puncture was not possible due to the anticoagulation, but this was performed four days into antibiotic treatment. CSF bacterial culture and 16S rDNA PCR were both negative at the time. Fourteen days after stopping antibiotic treatment, the patient was readmitted to the hospital for reduced consciousness.

As fever was noted, meningeal doses of vancomycin, cefepime, and ampicillin were commenced. Once again, a lumbar puncture could not be immediately performed due to anticoagulation. The scalp wound he previously sustained was noted to be close to the DBS lead. A brain CT with contrast showed streak artifact associated with DBS leads, but no acute intracranial pathology.

The CSF was hazy macroscopically, with a high WBC of 760×10⁶/L (63% lymphocytes, 11% lymphocytes, 25% monocytes/histiocytes, 1% basophils), RBC 28×10⁶/L, protein 58 mg/dL, glucose 48 mg/dL (corresponding serum glucose 75 mg/dL). CSF culture, HSV/VZV PCR, and 16S rDNA PCR were all negative. Three days after admission, the DBS was removed surgically, and bacterial culture of the prosthetic material was positive for Klebsiella aerogenes. The patient had complete resolution of the infection and a good clinical outcome.

At this point, a CSF sampled 2 days before the surgery removal of the infected hardware was retrospectively enrolled. CSF testing by mNGS was positive for Klebsiella aerogenes, which was further confirmed by digital PCR of the sequencing library (see FIG. 8A-B). Therefore, despite negative culture and 16S rDNA PCR, mNGS on the CSF was positive for the pathogen that was found to be infecting the DBS prosthetic material.

Case S89

Retrouterine fluid:
Culture (including anaerobic culture): negative
mNGS: multiple; top 5: Faecalibacterium prausnitzii, Eubacterium rectale, Akkermansia muciniphila, Acidaminococcus intestini, and Bifidobacterium adolescentis

Clinical adjudication: predominately anaerobic GI flora

A woman in her 20s with inflammatory bowel disease and past GI surgery presents with free air seen on an abdominal CT and was confirmed to have small bowel perforation during corrective surgery. One week after her operation, the patient continued to have leukocytosis and a CT scan showed a rectouterine fluid collection that was drained the next day. The rectouterine fluid was visually purulent (cloudy) and thick, but was negative on culture, including anaerobic culture. Testing by mNGS of this fluid drainage shows multiple, anaerobic, gastrointestinal bacteria: Faecalibacterium prausnitzii, Eubacterium rectale, Akkermansia muciniphila, Acidaminococcus intestini, and Bifidobacterium adolescentis as the top 5 most common organisms, whereas anaerobic culture results were negative. The primary surgical team started empiric antibiotics (piperacillin/tazobactam only) after drainage. CT imaging showed persisting rectouterine fluid collection a few days later, although slightly decreased and the patient's elevated white count normalized. Antibiotics were discontinued after nearly a week and the patient was discharged. In this case, mNGS could have suggested the addition of metronidazole to cover the undocumented anaerobic organisms.

Case S92

Bronchoalveolar lavage (BAL) fluid:
Gram stain and culture (including fungal culture): negative
BAL fluid mNGS: Aspergillus fumigatus (56 reads, 1.19 normalized RPM)

### Blood:

Plasma mNGS: Aspergillus fumigatus (1 read, 0.035 normalized RPM)
Serum beta-D-glucan: 316 picograms/mL (reference: <60)
Serum aspergillus galactomannan index: 4.5 (reference: <0.5)

Clinical adjudication: Aspergillus fumigatus (Probable by EORTC/MGS* international standards)

A man in his 60s with anaplastic large cell lymphoma and bladder cancer was admitted electively for chemotherapy. His clinical course was complicated by MRSA bacteremia and endocarditis from a PICC line source (treated with vancomycin) and ischemic bowel requiring primary resection and anastomosis.

A CT chest (without contrast) performed for persistent fevers and streaky opacities on CXR revealed multiple bilateral pulmonary nodules, nodular areas of consolidation, and a left pleural effusion, with unchanged supraclavicular, mediastinal, and hilar lymphadenopathy. Serum beta-D-glucan was raised at 316 picograms/mL (reference range <60) and serum aspergillus galactomannan index was raised at 4.501 (reference range <0.5).

The patient met the EORTC/MGS* criteria for probable invasive aspergillosis, and voriconazole was commenced.

*European Organization for Research and Treatment of Cancer

BAL and FNA of a pulmonary nodule were collected 3 days into voriconazole treatment. BAL Gram stain and cultures were negative. FNA revealed malignant lymphoma cells on cytology, consistent with the patient's known lymphoma, but also negative cultures.

At this point, the BAL sample was included in this series given that the patient was culture-negative but had a clinically probable invasive Aspergillus infection. mNGS of the BAL demonstrated the presence of Aspergillus fumigatus.

Voriconazole was changed to posaconazole after 8 days due to liver toxicity concerns. Follow-up of serum galactomannan index demonstrated a treatment response (falling to 0.24 mg/mL at 15 weeks of posaconazole treatment) and follow-up CT scans showed a continued decrease in size of the multiple pulmonary nodules representing resolving infection. The patient was subsequently discharged from the hospital with hematology and infectious diseases clinic follow-up.

Case S90

Blood culture: Streptococcus pneumoniae
Pleural fluid:
Culture: no growth
mNGS: Streptococcus pneumoniae

Clinical adjudication: Streptococcus pneumoniae

A woman in her 50s, with a history of a hematopoietic stem cell transplant 1 year ago, presented with fever, productive cough, and tachycardia, and was subsequently found to be blood culture positive for Streptococcus pneumoniae. CT imaging showed a left loculated pleural effusion. The effusion was drained, but the culture results of the pleural fluid were negative. The sample was enrolled into this study and mNGS results of the same pleural fluid showed Streptococcus pneumoniae as the top species at a normalized RPM of 57,856 and were more than 99.99% of all the microbial reads not classified into the same family or genus.

Case S91

Blood culture: Group A Streptococcus
Pleural fluid:
Culture: no growth
mNGS: Streptococcus pyogenes (Group A streptococcus)

Clinical adjudication: Streptococcus pyogenes

A woman in her 50s who presented with fever, malaise, and syncope in the setting of sepsis, was admitted with Group A Streptococcus bacteremia and pneumonia. She placed on ceftriaxone and quickly improved. She developed a complicated parapneumonic effusion (LDH (lactate dehydrogenase)>2700 U/L). The effusion was drained, but the culture of the pleural fluid was negative. The sample was enrolled into this study and mNGS of the same pleural fluid showed that Streptococcus pyogenes was the top species identified at a normalized RPM of 57,856 and were more than 99.99% of all the microbial reads not classified into the same family or genus.

Set 2: Additional Pathogens Incidentally Detected by Body Fluid mNGS Testing in Patients with Microbiologically Proven Infection

Case S31

Pleural fluid:
Culture: negative
16S rDNA PCR: Streptococcus mitis group
CSF mNGS Illumina: Klebsiella pneumoniae
CSF mNGS Nanopore: Klebsiella pneumoniae
Digital PCR: Klebsiella pneumoniae, negative for Streptococcus mitis
Sanger sequencing: Klebsiella pneumoniae
   Contralateral pleural fluid:
Culture: negative
mNGS Illumina: Klebsiella pneumoniae
mNGS Nanopore: Klebsiella pneumoniae
Digital PCR: Klebsiella pneumoniae, negative for Streptococcus mitis

Clinical adjudication: Klebsiella pneumoniae

A child with congenital CMV and myelodysplastic syndrome was admitted for chemotherapy. He developed febrile neutropenia with septic shock and coagulopathy. Despite empirical cefepime, the sepsis worsened with the development of ARDS and worsening abdominal distension, leading to an intensive care admission. His antibiotics were changed empirically to meropenem, ciprofloxacin, and vancomycin. Caspofungin was also initiated for antifungal cover.

CT imaging revealed necrotizing pneumonia involving all lobes of both lungs and moderate bilateral pleural effusions. Asymmetric enhancement of the small intestine may have indicated bowel inflammation/infection or septic shock physiology.

Blood, BAL, and pleural fluid were all negative on bacterial culture. The pleural fluid was exudative by Light's criteria. 16S rDNA PCR of the pleural fluid was positive for Streptococcus mitis group, with no other organisms detected.

Despite the specific 16S rDNA PCR result, a decision was made to continue the broad-spectrum antibiotic combination, including meropenem, to cover the range of possible organisms contributing to the necrotizing pneumonia. The patient improved clinically with no signs of sepsis after treatment. A chest CT verified resolution of the infection.

Pleural fluid mNGS by both Illumina and Nanopore sequencing showed Klebsiella pneumoniae. This was subsequently confirmed by digital PCR of both the sequencing library and the original DNA extract and Sanger sequencing of the DNA extract (see FIGS. 10B-E). Also, in a separate sample collection, library preparation, and sequencing run, the contralateral pleural fluid revealed only Klebsiella pneumoniae, which was similarly confirmed by digital PCR. Digital PCR of the original DNA extract from the bilateral pleural fluid targeting Streptococcus mitis was negative, suggesting that the organism was either a false positive contaminant in the 16S PCR or present at a low level for mNGS and digital PCR.

Case S65

Ascitic fluid:
Culture: no growth
16S rDNA PCR: Streptococcus pyogenes
Ascitic fluid mNGS: Streptococcus pyogenes

A previously healthy woman in her 30s presented to the hospital with diffuse abdominal pain, nausea, vomiting, watery diarrhea, fever, leukocytosis, and acute kidney injury four days after IUD placement. CT abdomen and pelvis demonstrated inflammation of the caecum, sigmoid colon, and rectum, with peritoneal enhancement and intra-abdominal ascites. Chlamydia and gonorrhea NAAT testing were negative.

A percutaneous drain was inserted five days into antibiotic treatment with piperacillin-tazobactam. The ascitic fluid showed WBC 14.375×10⁹/L (74% neutrophils, 5% lymphocytes, 21% others), a high total protein of 3.8 g/dL, and a serum albumin albumin gradient (SAAG) of 0.4 g/dL, consistent with infected ascites. Direct microbiological cultures of the ascitic fluid, however, yielded no growth.

This case was referred by a hospitalist physician and mNGS on the same ascitic fluid was positive for Streptococcus pyogenes. 16S PCR of a later ascitic fluid was previously sent and it was negative. The same ascitic fluid was then sent for 16S PCR that underwent mNGS and the 16S test was also positive for Streptococcus pyogenes. The patient was treated with 14 days of piperacillin-tazobactam, along with percutaneous drainage of subsequent loculated collections. She clinically improved was discharged from the hospital 20 days after admission.

Case S10

### Blood:

Culture at the hospital: negative
Culture at the previous outside hospital: Haemophilus influenzae
Plasma mNGS: Haemophilus influenzae
   Bronchoalveolar lavage (BAL) fluid:
Culture: negative
16S rDNA PCR: Haemophilus influenzae
BAL mNGS: Haemophilus influenzae
Second BAL 2 days after the first BAL: Haemophilus influenzae

A woman in her 30s who was a smoker and previous intravenous drug user presented with one week of productive cough and dyspnea. She developed type 1 respiratory failure and cardiogenic shock, requiring intubation, ventilation, and ECMO support. She was then transferred from the outside hospital to a tertiary care hospital. CT chest revealed bilateral upper lobe consolidation with patchy regions of nodular consolidation throughout the remaining lung fields, with diffuse mediastinal and hilar lymphadenopathy. The blood cultures and BAL cultures collected three days after the initiation of antibiotics at the outside hospital were all negative.

This case was referred by an infectious disease specialist and mNGS of the BAL fluid was positive for Haemophilus influenzae. One of 2 blood cultures prior to antibiotics at an outside hospital was also positive for Haemophilus influenzae. Subsequent 16S rDNA PCR was positive for Haemophilus influenzae.

The patient was commenced empirically on ceftriaxone, vancomycin, and azithromycin. These were subsequently changed to ceftriaxone monotherapy based on Haemophilus influenzae sensitivity results. Her workup revealed a new diagnosis of B-cell acute lymphoblastic leukemia. The patient improved clinically, completed induction chemotherapy, and has been disease-free for over a year.

Case S42

### Blood:

Blood culture: Escherichia coli
Plasma mNGS: Fusobacterium nucleatum and Escherichia coli

### Abscess:

Culture: Escherichia coli
mNGS: Fusobacterium nucleatum and Escherichia coli

A woman in her 30s with a history of chronic endometriosis and laparotomy for ruptured appendicitis and tubo-ovarian abscess two months prior to admission, was readmitted for severe lower abdominal pain, vaginal bleeding, nausea, low-grade fevers, and chills. CT abdomen and pelvis showed multiple loculated abdominal and pelvic abscesses (the largest measuring 9×7×17 cm) interspersed between bowel loops and mesentery—these could not be surgically drained due to the dense adhesions and bowel loops surrounding the fluid collections. Piperacillin-tazobactam was commenced empirically and an abdominal drain was placed into the large abscess. Both blood cultures from admission and abscess fluid cultures grew pan-sensitive Escherichia coli only.

Plasma and abscess fluid mNGS both showed DNA reads to Fusobacterium nucleatum and Escherichia coli. Follow-up CT two weeks later showed resolution of multiple abscesses, with minimal residual collections remaining.

Fusobacterium nucleatum is an anaerobe commonly found in polymicrobial intra-abdominal abscesses. This was detected by mNGS and was not detected by conventional bacterial culture.

Case S64

AV graft tissue culture: negative

Peri-graft swab:
culture with follow-up 16S identification: Mycoplasma hominis (22 days time to result)
mNGS: Mycoplasma hominis

A woman in her 50s presented with fever and tenderness in the area over a Polytetrafluoroethylene (PTFE) arteriovenous graft. She had a background of end-stage renal failure with a renal transplantation two months prior to admission. Intra-operative findings during graft excision revealed that the graft was completely thrombosed, with surrounding purulent fluid and extension of the infection along the graft to disrupt the arterial anastomosis.

AV graft tissue cultures were negative, but a peri-graft swab grew pinpoint colonies of gram-negative rods after 6 days. Identification of the colonies was difficult as MALDI-ToF (matrix-associated laser desorption/ionization—time of flight) and biochemical testing were inconclusive. Send-out 16S sequencing eventually identified the colonies as Mycoplasma hominis 16 additional days later. mNGS from the original pen-graft swab (available on day 0) was also positive for Mycoplasma hominis. Nanopore real-time sequencing took less than 10 minutes for organism identification after the initiation of sequencing.

The patient was discharged back to her referring hospital before final culture results were available, as the Mycoplasma hominis required 16S PCR for identification. This is a case where an earlier result (such as by mNGS) would have had an impact on clinical management as vancomycin is an ineffective treatment for Mycoplasma hominis.

It will be understood that various modifications may be made without departing from the spirit and scope of this disclosure. Accordingly, other embodiments are within the scope of the following claims.

1. An oligonucleotide comprising barcodes for use in multiple types of next generation sequencing technologies, the barcodes comprising
at least about 18 to about 39 nucleotides in length having a first nucleotide domain and at least one second nucleotide domain;
wherein the first nucleotide domain comprises 4-9 nucleotides (4-9mer) of the barcode located at either end of the barcode and wherein the 4-9mers are compatible with a next generation sequencing technology that utilizes bridge amplification,
wherein the second nucleotide domain comprises 14-35 nucleotides (14-35mer) of the barcode and wherein the 14-35mers are compatible with a next generation sequencing that utilizes nanopores,
wherein at least a minimum Levenshtein distance between a pair of 4-9mers is utilized, and
wherein the Levenshtein distance has been maximized between a pair of barcodes in order to minimize barcode “crosstalk”.

2. The oligonucleotide of claim 1, wherein the oligonucleotide further comprises a flow cell attachment domain.

3. The oligonucleotide of claim 2, wherein the flow cell attachment domain comprises a sequence selected from SEQ ID NO:1, 2, 3 or 4.

4. The oligonucleotide of claim 1, further comprising a sequencing primer binding domain.

5. The oligonucleotide of claim 1, wherein the barcode is comprised of the 4-9mer and the second domain comprises 3 sets of 10 mers that when concatenated together form a 34-39mer, wherein the last nucleotide is removed to form the 33-38mer barcode.

6. The oligonucleotide of claim 1, wherein the oligonucleotide comprises a sequence selected from any one of SEQ ID Nos: 226-416 and 417.

7. The oligonucleotide of claim 1, wherein the oligonucleotide consists of 47-80 nucleotides.

8. The oligonucleotide of claim 1, wherein the oligonucleotide is 62-83 nucleotides in length.

9. An oligonucleotide barcode sequence for use in multiple types of next generation sequencing, wherein the oligonucleotide barcode is about 24 to 39 nucleotides in length and comprises a first oligonucleotide barcode domain of about 4-9 nucleotides (4-9mer) at the 5′ or 3′ end of the oligonucleotide barcode and a second oligonucleotide barcode domain of about 10-29 nucleotides in length operably linked to the first oligonucleotide barcode domain, wherein the Levenshtein distance has been maximized between a pair of oligonucleotide barcodes in order to minimize barcode “crosstalk”;
wherein the first oligonucleotide barcode domain is compatible with next generation sequencing using bridge amplification;
wherein the second oligonucleotide barcode domain is compatible with next generation sequencing using nanopores; and
wherein the oligonucleotide has a minimum Levenshtein distance between a pair of 4-9mers.

10. The oligonucleotide barcode sequence of claim 9, wherein the barcode is about 36-39 nucleotides in length.

11. The oligonucleotide barcode sequence of claim 9, wherein the oligonucleotide comprises a sequence selected from the group consisting of SEQ ID Nos: 226-416 and 417.

12. A set of oligonucleotides comprising barcodes of claim 1.

13. The set of oligonucleotides of claim 12, wherein each barcode is located between a pair of sequencing adaptors.

14. The set of oligonucleotides of claim 13, wherein the pair of sequencing adaptors have sequences selected from (i) or (ii):
(i)
(SEQ ID NO: 1)
CAAGCAGAAGACGGCATACGAGAT,
and
(SEQ ID NO: 2)
GTGACTGGAGTTCAGACGTGTGCTCTTCCGATC*T;
or
(ii)
(SEQ ID NO: 3)
AATGATACGGCGACCACCGAGATCTACAC,
and
(SEQ ID NO: 4)
ACACTCTTTCCCTACACGACGCTCTTCCGATC*T,
wherein * indicates a phosphorothioate bond between the nucleotides.

15. The set of oligonucleotides of claim 13, wherein the set of oligonucleotides are PCR primers used for sequencing library barcoding.

16. A sequencing library comprising the set of barcodes of claim 1.

17. The sequencing library of claim 16, wherein the sequencing library is used for an application selected from: pathogen discovery, environmental metagenomics, de novo genome assembly, whole-exome sequencing, transcriptomics sequencing, targeted gene panel sequencing or whole-genome resequencing.

18. A method for rapid pathogen detection in a sample using metagenomic next-generation sequencing (mNGS), comprising:
obtaining one or more samples comprising cell-free DNA (cfDNA);
generating a plurality of sequencing reads comprising a barcode from the set of barcodes of claim 12 using next-generation sequencing;
performing metagenomic analysis on the plurality of sequencing read data using a clinical bioinformatics software pipeline that can rapidly analyze sequencing read data for pathogenic DNA;
determining and identifying pathogen(s) in the one or more samples based upon the metagenomic analysis of the sequencing read data.

19. The method of claim 18, wherein the one or more samples comprises a body fluid sample from a subject.

20. (canceled)

21. The method of claim 19, wherein the body fluid sample is selected from cerebrospinal fluid, urine, semen, pericardial fluid, pleural fluid, peritoneal fluid, synovial fluid, amniotic fluid, fetal fibronectin, saliva, sweat, eye vitreous humor, eye aqueous humor, bronchoalveolar lavage fluid, breast milk, bile, and ascites fluid.

22. The method of claim 21, wherein the one or more samples further comprise a blood serum sample.

23. The method of claim 18, wherein the next-generation sequencing comprises (i) sequencing technology that utilizes bridge amplification, or (ii) sequencing technology that utilizes nanopores; or (iii) a combination of (i) and (ii).

24-25. (canceled)

26. The method of claim 18, wherein the clinical bioinformatics software pipeline that can rapidly analyze sequencing read data for pathogenic DNA is SURPI+ or SURPIrt.

27. The method of claim 18, wherein the pathogen(s) comprise one or more pathogenic bacteria, or one or more pathogenic fungi.

28. (canceled)

29. A set of paired 37mer barcodes comprising dual indexes that are configured for dual use in multiple types of next generation sequencing technologies,
wherein the Levenshtein distance has been maximized between each pair of 37mer barcodes in order to minimize barcode “crosstalk”;
wherein the first 8 nucleotides (8mer) of each pair of 37mer barcodes is compatible with a next generation sequencing technology that utilizes bridge amplification, and wherein at least a minimum Levenshtein distance between each pair of 8mers is utilized;
wherein at least a minimum Levenshtein distance between each pair of 37mers barcodes is used so that the 37mer barcode is compatible with a next generation sequencing technology that utilizes nanopores.


SEQUENCE LISTING (419 sequences)

SEQ ID NO: 1 - DNA, 24 bp, Artificial Sequence
  Description of Artificial Sequence Synthetic oligonucleotide
caagcagaagacggcatacgagat

SEQ ID NO: 2 - DNA, 34 bp, Artificial Sequence
  Description of Artificial Sequence Synthetic oligonucleotide
gtgactggagttcagacgtgtgctcttccgatct

SEQ ID NO: 3 - DNA, 29 bp, Artificial Sequence
  Description of Artificial Sequence Synthetic oligonucleotide
aatgatacggcgaccaccgagatctacac

SEQ ID NO: 4 - DNA, 33 bp, Artificial Sequence
  Description of Artificial Sequence Synthetic oligonucleotide
acactctttccctacacgacgctcttccgatct

SEQ ID NO: 5 - DNA, 95 bp, Artificial Sequence
  Description of Artificial Sequence Synthetic oligonucleotide
caagcagaagacggcatacgagatctccgtatcttctccatttgttgtgcagaaatggctcgtgactggagttcagacgtgtgctcttccgatct

SEQ ID NO: 6 - DNA, 95 bp, Artificial Sequence
  Description of Artificial Sequence Synthetic oligonucleotide
caagcagaagacggcatacgagatgagactctatacctcctcctctatacgttcgcttcatgtgactggagttcagacgtgtgctcttccgatct

SEQ ID NO: 7 - DNA, 95 bp, Artificial Sequence
  Description of Artificial Sequence Synthetic oligonucleotide
caagcagaagacggcatacgagataccgtggagtcataagcttgacctcgccacatgtctggtgactggagttcagacgtgtgctcttccgatct

SEQ ID NO: 8 - DNA, 95 bp, Artificial Sequence
  Description of Artificial Sequence Synthetic oligonucleotide
caagcagaagacggcatacgagatccggactgatgaccggattaagttcgcagtcaccgaagtgactggagttcagacgtgtgctcttccgatct

SEQ ID NO: 9 - DNA, 95 bp, Artificial Sequence
  Description of Artificial Sequence Synthetic oligonucleotide
caagcagaagacggcatacgagatgctctagccgtcactctttatcctcacactactggctgtgactggagttcagacgtgtgctcttccgatct

SEQ ID NO: 10 - DNA, 95 bp, Artificial Sequence
  Description of Artificial Sequence Synthetic oligonucleotide
caagcagaagacggcatacgagatcatctgttctcgttacacagagctgccaagtacagtggtgactggagttcagacgtgtgctcttccgatct

SEQ ID NO: 11 - DNA, 95 bp, Artificial Sequence
  Description of Artificial Sequence Synthetic oligonucleotide
caagcagaagacggcatacgagatccgttctcttgagcgcattatagaagcgccaagatcggtgactggagttcagacgtgtgctcttccgatct

SEQ ID NO: 12 - DNA, 95 bp, Artificial Sequence
  Description of Artificial Sequence Synthetic oligonucleotide
caagcagaagacggcatacgagatatcgtggtcgcttaccgttgtcaaggacaagctgatggtgactggagttcagacgtgtgctcttccgatct

SEQ ID NO: 13 - DNA, 95 bp, Artificial Sequence
  Description of Artificial Sequence Synthetic oligonucleotide
caagcagaagacggcatacgagatagagcaatgacgatatgttcttcggcatggtagtagcgtgactggagttcagacgtgtgctcttccgatct

SEQ ID NO: 14 - DNA, 95 bp, Artificial Sequence
  Description of Artificial Sequence Synthetic oligonucleotide
caagcagaagacggcatacgagatgtcggtatcttatgtgcagctgttcgaccggtgtacagtgactggagttcagacgtgtgctcttccgatct

SEQ ID NO: 15 - DNA, 95 bp, Artificial Sequence
  Description of Artificial Sequence Synthetic oligonucleotide
caagcagaagacggcatacgagatgtgacaactgagtgactttatgctgccggctctcaacgtgactggagttcagacgtgtgctcttccgatct

SEQ ID NO: 16 - DNA, 95 bp, Artificial Sequence
  Description of Artificial Sequence Synthetic oligonucleotide
caagcagaagacggcatacgagatgaacgattccaacgtaattgtgttgtcctcaaggagtgtgactggagttcagacgtgtgctcttccgatct

SEQ ID NO: 17 - DNA, 95 bp, Artificial Sequence
  Description of Artificial Sequence Synthetic oligonucleotide
caagcagaagacggcatacgagatggttcgcaggcaggtcacaacaccgttctttacggaggtgactggagttcagacgtgtgctcttccgatct

SEQ ID NO: 18 - DNA, 95 bp, Artificial Sequence
  Description of Artificial Sequence Synthetic oligonucleotide
caagcagaagacggcatacgagattgttctcccaattgtagttgctccacatatctgtgcagtgactggagttcagacgtgtgctcttccgatct

SEQ ID NO: 19 - DNA, 95 bp, Artificial Sequence
  Description of Artificial Sequence Synthetic oligonucleotide
caagcagaagacggcatacgagatatggccgtcagttgtgtaactgtgacctctctgaggtgtgactggagttcagacgtgtgctcttccgatct

SEQ ID NO: 20 - DNA, 95 bp, Artificial Sequence
  Description of Artificial Sequence Synthetic oligonucleotide
caagcagaagacggcatacgagattaagcgtccatgattgatgctaatgttccctctacctgtgactggagttcagacgtgtgctcttccgatct

SEQ ID NO: 21 - DNA, 95 bp, Artificial Sequence
  Description of Artificial Sequence Synthetic oligonucleotide
caagcagaagacggcatacgagattaactgtcttaggctaattctggactagcatgttcgcgtgactggagttcagacgtgtgctcttccgatct

SEQ ID NO: 22 - DNA, 95 bp, Artificial Sequence
  Description of Artificial Sequence Synthetic oligonucleotide
caagcagaagacggcatacgagattggccacggtcattgtacaggtaccgcatagtcctaggtgactggagttcagacgtgtgctcttccgatct

SEQ ID NO: 23 - DNA, 95 bp, Artificial Sequence
  Description of Artificial Sequence Synthetic oligonucleotide
caagcagaagacggcatacgagattagcgaaagatgccgacgaatagcttgcgggtcagatgtgactggagttcagacgtgtgctcttccgatct

SEQ ID NO: 24 - DNA, 95 bp, Artificial Sequence
  Description of Artificial Sequence Synthetic oligonucleotide
caagcagaagacggcatacgagatccatacggcctggatcaccaatatggagccgtccatagtgactggagttcagacgtgtgctcttccgatct

SEQ ID NO: 25 - DNA, 95 bp, Artificial Sequence
  Description of Artificial Sequence Synthetic oligonucleotide
caagcagaagacggcatacgagatacgtctcatgccacctgttggccatgcagttctctgggtgactggagttcagacgtgtgctcttccgatct

SEQ ID NO: 26 - DNA, 95 bp, Artificial Sequence
  Description of Artificial Sequence Synthetic oligonucleotide
caagcagaagacggcatacgagatcagagacatacatggctccaagttcaggcgtccttctgtgactggagttcagacgtgtgctcttccgatct

SEQ ID NO: 27 - DNA, 95 bp, Artificial Sequence
  Description of Artificial Sequence Synthetic oligonucleotide
caagcagaagacggcatacgagatcgagcttctcgaccataccacatcgacatttccgcaagtgactggagttcagacgtgtgctcttccgatct

SEQ ID NO: 28 - DNA, 95 bp, Artificial Sequence
  Description of Artificial Sequence Synthetic oligonucleotide
caagcagaagacggcatacgagataaccgaggatagcaccgtaccatccatctaggataccgtgactggagttcagacgtgtgctcttccgatct

SEQ ID NO: 29 - DNA, 95 bp, Artificial Sequence
  Description of Artificial Sequence Synthetic oligonucleotide
caagcagaagacggcatacgagattatggcggctgcgattctggagatatgtgcgctatgtgtgactggagttcagacgtgtgctcttccgatct

SEQ ID NO: 30 - DNA, 95 bp, Artificial Sequence
  Description of Artificial Sequence Synthetic oligonucleotide
caagcagaagacggcatacgagatgagagagtgaccaagtaccactagttaagcagctactgtgactggagttcagacgtgtgctcttccgatct

SEQ ID NO: 31 - DNA, 95 bp, Artificial Sequence
  Description of Artificial Sequence Synthetic oligonucleotide
caagcagaagacggcatacgagatctggtccagcttctctaacaagttggttcgagaagtcgtgactggagttcagacgtgtgctcttccgatct

SEQ ID NO: 32 - DNA, 95 bp, Artificial Sequence
  Description of Artificial Sequence Synthetic oligonucleotide
caagcagaagacggcatacgagatgtcacacgctcaccgtttggacctgttggttcgcgtagtgactggagttcagacgtgtgctcttccgatct

SEQ ID NO: 33 - DNA, 95 bp, Artificial Sequence
  Description of Artificial Sequence Synthetic oligonucleotide
caagcagaagacggcatacgagatggtcctatcgtgtctaattccgccttggattgaggcagtgactggagttcagacgtgtgctcttccgatct

SEQ ID NO: 34 - DNA, 95 bp, Artificial Sequence
  Description of Artificial Sequence Synthetic oligonucleotide
caagcagaagacggcatacgagatagttcactccagatctgttgcgcaccttgcttcacaggtgactggagttcagacgtgtgctcttccgatct

SEQ ID NO: 35 - DNA, 95 bp, Artificial Sequence
  Description of Artificial Sequence Synthetic oligonucleotide
caagcagaagacggcatacgagatacggtgctgtagtcagcttagcacttcggtaccacctgtgactggagttcagacgtgtgctcttccgatct

SEQ ID NO: 36 - DNA, 95 bp, Artificial Sequence
  Description of Artificial Sequence Synthetic oligonucleotide
caagcagaagacggcatacgagataagattcatacgacagttaagctagagacttcctcgtgtgactggagttcagacgtgtgctcttccgatct

SEQ ID NO: 37 - DNA, 95 bp, Artificial Sequence
  Description of Artificial Sequence Synthetic oligonucleotide
caagcagaagacggcatacgagatgacgtgacggctgcaatttgagcgttgtttacctctcgtgactggagttcagacgtgtgctcttccgatct

SEQ ID NO: 38 - DNA, 95 bp, Artificial Sequence
  Description of Artificial Sequence Synthetic oligonucleotide
caagcagaagacggcatacgagatcaactactcggattgagttgtacgtccgccgagttgtgtgactggagttcagacgtgtgctcttccgatct

SEQ ID NO: 39 - DNA, 95 bp, Artificial Sequence
  Description of Artificial Sequence Synthetic oligonucleotide
caagcagaagacggcatacgagatacggcagtggtacaactttggtagaggttggtggattgtgactggagttcagacgtgtgctcttccgatct

SEQ ID NO: 40 - DNA, 95 bp, Artificial Sequence
  Description of Artificial Sequence Synthetic oligonucleotide
caagcagaagacggcatacgagatcgattctgccgtgaactttcgactatcctgatggagagtgactggagttcagacgtgtgctcttccgatct

SEQ ID NO: 41 - DNA, 95 bp, Artificial Sequence
  Description of Artificial Sequence Synthetic oligonucleotide
caagcagaagacggcatacgagatgtcgtagacggacctgttgaatgcctcaacgaaggttgtgactggagttcagacgtgtgctcttccgatct

SEQ ID NO: 42 - DNA, 95 bp, Artificial Sequence
  Description of Artificial Sequence Synthetic oligonucleotide
caagcagaagacggcatacgagatgtaactgagcaactgcctcgaattccaagtgcggtaagtgactggagttcagacgtgtgctcttccgatct

SEQ ID NO: 43 - DNA, 95 bp, Artificial Sequence
  Description of Artificial Sequence Synthetic oligonucleotide
caagcagaagacggcatacgagattgtccaactaatgctctttgtagccaatgtcacttcggtgactggagttcagacgtgtgctcttccgatct

SEQ ID NO: 44 - DNA, 95 bp, Artificial Sequence
  Description of Artificial Sequence Synthetic oligonucleotide
caagcagaagacggcatacgagatttcggacggttcattagttcacaagcggatgaagaccgtgactggagttcagacgtgtgctcttccgatct

SEQ ID NO: 45 - DNA, 95 bp, Artificial Sequence
  Description of Artificial Sequence Synthetic oligonucleotide
caagcagaagacggcatacgagatggttaagcatgcctctattcctgatctgacagcatcagtgactggagttcagacgtgtgctcttccgatct

SEQ ID NO: 46 - DNA, 95 bp, Artificial Sequence
  Description of Artificial Sequence Synthetic oligonucleotide
caagcagaagacggcatacgagataatgtactgtgactctttaccagagaccgccttaacggtgactggagttcagacgtgtgctcttccgatct

SEQ ID NO: 47 - DNA, 95 bp, Artificial Sequence
  Description of Artificial Sequence Synthetic oligonucleotide
caagcagaagacggcatacgagatggttaccatgcaccaatttgatggaacctgtctcacagtgactggagttcagacgtgtgctcttccgatct

SEQ ID NO: 48 - DNA, 95 bp, Artificial Sequence
  Description of Artificial Sequence Synthetic oligonucleotide
caagcagaagacggcatacgagattcgtagaacctccacagttgtctcttgtgacaccttcgtgactggagttcagacgtgtgctcttccgatct

SEQ ID NO: 49 - DNA, 95 bp, Artificial Sequence
  Description of Artificial Sequence Synthetic oligonucleotide
caagcagaagacggcatacgagatgcatggaacacctcgttttctcaggttggctgcactagtgactggagttcagacgtgtgctcttccgatct

SEQ ID NO: 50 - DNA, 95 bp, Artificial Sequence
  Description of Artificial Sequence Synthetic oligonucleotide
caagcagaagacggcatacgagataagatcgtatagcgctcttcggtgttccggctaggaagtgactggagttcagacgtgtgctcttccgatct

SEQ ID NO: 51 - DNA, 95 bp, Artificial Sequence
  Description of Artificial Sequence Synthetic oligonucleotide
caagcagaagacggcatacgagatacggattcctaacaggtaagcgtaagcggttgttgccgtgactggagttcagacgtgtgctcttccgatct

SEQ ID NO: 52 - DNA, 95 bp, Artificial Sequence
  Description of Artificial Sequence Synthetic oligonucleotide
caagcagaagacggcatacgagatatccatatggcgttggaccgtcgttatcagccgatatgtgactggagttcagacgtgtgctcttccgatct

SEQ ID NO: 53 - DNA, 95 bp, Artificial Sequence
  Description of Artificial Sequence Synthetic oligonucleotide
caagcagaagacggcatacgagattaaccattcgcttcattttccgaacaggtccgacttagtgactggagttcagacgtgtgctcttccgatct

SEQ ID NO: 54 - DNA, 95 bp, Artificial Sequence
  Description of Artificial Sequence Synthetic oligonucleotide
caagcagaagacggcatacgagatgagtgtttgcctatgtttacgaacgccttttccgttcgtgactggagttcagacgtgtgctcttccgatct

SEQ ID NO: 55 - DNA, 95 bp, Artificial Sequence
  Description of Artificial Sequence Synthetic oligonucleotide
caagcagaagacggcatacgagattactaacccgttcttcgttctggttgacatcggagaagtgactggagttcagacgtgtgctcttccgatct

SEQ ID NO: 56 - DNA, 95 bp, Artificial Sequence
  Description of Artificial Sequence Synthetic oligonucleotide
caagcagaagacggcatacgagattatacaatagtgattgcccgaggatcgaaccaaccaagtgactggagttcagacgtgtgctcttccgatct

SEQ ID NO: 57 - DNA, 95 bp, Artificial Sequence
  Description of Artificial Sequence Synthetic oligonucleotide
caagcagaagacggcatacgagatgttggctggaggcgaatttctccgctgtcaggatcgagtgactggagttcagacgtgtgctcttccgatct

SEQ ID NO: 58 - DNA, 95 bp, Artificial Sequence
  Description of Artificial Sequence Synthetic oligonucleotide
caagcagaagacggcatacgagattcttggagttcatcacattgcaataccactccatggtgtgactggagttcagacgtgtgctcttccgatct

SEQ ID NO: 59 - DNA, 95 bp, Artificial Sequence
  Description of Artificial Sequence Synthetic oligonucleotide
caagcagaagacggcatacgagatgactaacgagcgatcactaccatggcgacttgaacgcgtgactggagttcagacgtgtgctcttccgatct

SEQ ID NO: 60 - DNA, 95 bp, Artificial Sequence
  Description of Artificial Sequence Synthetic oligonucleotide
caagcagaagacggcatacgagatcgtctgtccagttgacctggtctggtatttaccatgcgtgactggagttcagacgtgtgctcttccgatct

SEQ ID NO: 61 - DNA, 95 bp, Artificial Sequence
  Description of Artificial Sequence Synthetic oligonucleotide
caagcagaagacggcatacgagatcctgtgactactatccataactcaacgcaagtacggtgtgactggagttcagacgtgtgctcttccgatct

SEQ ID NO: 62 - DNA, 95 bp, Artificial Sequence
  Description of Artificial Sequence Synthetic oligonucleotide
caagcagaagacggcatacgagatagaccaatggcacgagacagaattggcgggttacctcgtgactggagttcagacgtgtgctcttccgatct

SEQ ID NO: 63 - DNA, 95 bp, Artificial Sequence
  Description of Artificial Sequence Synthetic oligonucleotide
caagcagaagacggcatacgagatgtgaagtcttgtcttctttcgaaggtatctagagagggtgactggagttcagacgtgtgctcttccgatct

SEQ ID NO: 64 - DNA, 95 bp, Artificial Sequence
  Description of Artificial Sequence Synthetic oligonucleotide
caagcagaagacggcatacgagatgatgcaactggtagagcttccactccgtaaatccgtggtgactggagttcagacgtgtgctcttccgatct

SEQ ID NO: 65 - DNA, 95 bp, Artificial Sequence
  Description of Artificial Sequence Synthetic oligonucleotide
caagcagaagacggcatacgagatattgaggcaactgaacctacgacattcacggatgactgtgactggagttcagacgtgtgctcttccgatct

SEQ ID NO: 66 - DNA, 95 bp, Artificial Sequence
  Description of Artificial Sequence Synthetic oligonucleotide
caagcagaagacggcatacgagattggtgtccttaagtgagcaagacctagattgtggttcgtgactggagttcagacgtgtgctcttccgatct

SEQ ID NO: 67 - DNA, 95 bp, Artificial Sequence
  Description of Artificial Sequence Synthetic oligonucleotide
caagcagaagacggcatacgagatcagttgatatgcacacattggtgactaagagatgcgtgtgactggagttcagacgtgtgctcttccgatct

SEQ ID NO: 68 - DNA, 95 bp, Artificial Sequence
  Description of Artificial Sequence Synthetic oligonucleotide
caagcagaagacggcatacgagatcagacattggtactggcttggctagtcgaccaagacagtgactggagttcagacgtgtgctcttccgatct

SEQ ID NO: 69 - DNA, 95 bp, Artificial Sequence
  Description of Artificial Sequence Synthetic oligonucleotide
caagcagaagacggcatacgagatgcttaatggcggatgaattgcagtcgtggggtaccatgtgactggagttcagacgtgtgctcttccgatct

SEQ ID NO: 70 - DNA, 95 bp, Artificial Sequence
  Description of Artificial Sequence Synthetic oligonucleotide
caagcagaagacggcatacgagatcctaggatcattgccgctcaccgccagaattggacaggtgactggagttcagacgtgtgctcttccgatct

SEQ ID NO: 71 - DNA, 95 bp, Artificial Sequence
  Description of Artificial Sequence Synthetic oligonucleotide
caagcagaagacggcatacgagatgacggtgctcctacttattgtcaccagccaactcgaggtgactggagttcagacgtgtgctcttccgatct

SEQ ID NO: 72 - DNA, 95 bp, Artificial Sequence
  Description of Artificial Sequence Synthetic oligonucleotide
caagcagaagacggcatacgagatcagtgcccgtcttatacccgtgtattatgagcgagaagtgactggagttcagacgtgtgctcttccgatct

SEQ ID NO: 73 - DNA, 95 bp, Artificial Sequence
  Description of Artificial Sequence Synthetic oligonucleotide
caagcagaagacggcatacgagataatcgctatcggagaggttcgcagaagcatcggttaggtgactggagttcagacgtgtgctcttccgatct

SEQ ID NO: 74 - DNA, 95 bp, Artificial Sequence
  Description of Artificial Sequence Synthetic oligonucleotide
caagcagaagacggcatacgagattcagtcgaggccaatgcttcttcctagagttgtccgtgtgactggagttcagacgtgtgctcttccgatct

SEQ ID NO: 75 - DNA, 95 bp, Artificial Sequence
  Description of Artificial Sequence Synthetic oligonucleotide
caagcagaagacggcatacgagatacgtgaaggtcaacctcttacctactggcactgtacggtgactggagttcagacgtgtgctcttccgatct

SEQ ID NO: 76 - DNA, 95 bp, Artificial Sequence
  Description of Artificial Sequence Synthetic oligonucleotide
caagcagaagacggcatacgagataagccatgaggtgctcattatcggcctcccgtgatcagtgactggagttcagacgtgtgctcttccgatct

SEQ ID NO: 77 - DNA, 95 bp, Artificial Sequence
  Description of Artificial Sequence Synthetic oligonucleotide
caagcagaagacggcatacgagatgtgttagtgacttcgctccaacaagctaccagtcaaggtgactggagttcagacgtgtgctcttccgatct

SEQ ID NO: 78 - DNA, 95 bp, Artificial Sequence
  Description of Artificial Sequence Synthetic oligonucleotide
caagcagaagacggcatacgagatgactgcccggattcatcggcacttaagaactaacaccgtgactggagttcagacgtgtgctcttccgatct

SEQ ID NO: 79 - DNA, 95 bp, Artificial Sequence
  Description of Artificial Sequence Synthetic oligonucleotide
caagcagaagacggcatacgagatctgcatctggagaatacttggcagcatattagcggttgtgactggagttcagacgtgtgctcttccgatct

SEQ ID NO: 80 - DNA, 95 bp, Artificial Sequence
  Description of Artificial Sequence Synthetic oligonucleotide
caagcagaagacggcatacgagattaacaccaccgcttgtcttcaatggcagtggtcttgtgtgactggagttcagacgtgtgctcttccgatct

SEQ ID NO: 81 - DNA, 95 bp, Artificial Sequence
  Description of Artificial Sequence Synthetic oligonucleotide
caagcagaagacggcatacgagattggcattgtaagctgtcttaaggacgttcattcggacgtgactggagttcagacgtgtgctcttccgatct

SEQ ID NO: 82 - DNA, 95 bp, Artificial Sequence
  Description of Artificial Sequence Synthetic oligonucleotide
caagcagaagacggcatacgagatcggtgtgtcgcgcaatgttctcgttccttggaactgtgtgactggagttcagacgtgtgctcttccgatct

SEQ ID NO: 83 - DNA, 95 bp, Artificial Sequence
  Description of Artificial Sequence Synthetic oligonucleotide
caagcagaagacggcatacgagattgtaggaacgtagcatattccgattgttgctgtctctgtgactggagttcagacgtgtgctcttccgatct

SEQ ID NO: 84 - DNA, 95 bp, Artificial Sequence
  Description of Artificial Sequence Synthetic oligonucleotide
caagcagaagacggcatacgagattcactgacagtgcagcttgaggccgaagtttatcggcgtgactggagttcagacgtgtgctcttccgatct

SEQ ID NO: 85 - DNA, 95 bp, Artificial Sequence
  Description of Artificial Sequence Synthetic oligonucleotide
caagcagaagacggcatacgagatacactggaggtacgtatacgtatccgaggttgctccagtgactggagttcagacgtgtgctcttccgatct

SEQ ID NO: 86 - DNA, 95 bp, Artificial Sequence
  Description of Artificial Sequence Synthetic oligonucleotide
caagcagaagacggcatacgagatctctctagtggcctacaccagcttctagtttccactggtgactggagttcagacgtgtgctcttccgatct

SEQ ID NO: 87 - DNA, 95 bp, Artificial Sequence
  Description of Artificial Sequence Synthetic oligonucleotide
caagcagaagacggcatacgagatgacttatacgtgcgaagttcaagattgcccaggcattgtgactggagttcagacgtgtgctcttccgatct

SEQ ID NO: 88 - DNA, 95 bp, Artificial Sequence
  Description of Artificial Sequence Synthetic oligonucleotide
caagcagaagacggcatacgagattcacgccgttctatggcccggttacaattacgacactgtgactggagttcagacgtgtgctcttccgatct

SEQ ID NO: 89 - DNA, 95 bp, Artificial Sequence
  Description of Artificial Sequence Synthetic oligonucleotide
caagcagaagacggcatacgagataggtggaatgacagcgcttcataacaccgtcaagtgggtgactggagttcagacgtgtgctcttccgatct

SEQ ID NO: 90 - DNA, 95 bp, Artificial Sequence
  Description of Artificial Sequence Synthetic oligonucleotide
caagcagaagacggcatacgagatttgagcatgaactgcattcagtgaagtcgttggcactgtgactggagttcagacgtgtgctcttccgatct

SEQ ID NO: 91 - DNA, 95 bp, Artificial Sequence
  Description of Artificial Sequence Synthetic oligonucleotide
caagcagaagacggcatacgagatcaatcagccgtatggtcggagacactccttatgcaccgtgactggagttcagacgtgtgctcttccgatct

SEQ ID NO: 92 - DNA, 95 bp, Artificial Sequence
  Description of Artificial Sequence Synthetic oligonucleotide
caagcagaagacggcatacgagattatcaacatgcttgctttacggccaacatggagattggtgactggagttcagacgtgtgctcttccgatct

SEQ ID NO: 93 - DNA, 95 bp, Artificial Sequence
  Description of Artificial Sequence Synthetic oligonucleotide
caagcagaagacggcatacgagattatggagcgttgcttaatcagagcaacttccgataacgtgactggagttcagacgtgtgctcttccgatct

SEQ ID NO: 94 - DNA, 95 bp, Artificial Sequence
  Description of Artificial Sequence Synthetic oligonucleotide
caagcagaagacggcatacgagattgcattagttcgcactctgcctatactcggaagttccgtgactggagttcagacgtgtgctcttccgatct

SEQ ID NO: 95 - DNA, 95 bp, Artificial Sequence
  Description of Artificial Sequence Synthetic oligonucleotide
caagcagaagacggcatacgagattgaatggataggagccattgccttccgatgcttcagagtgactggagttcagacgtgtgctcttccgatct

SEQ ID NO: 96 - DNA, 95 bp, Artificial Sequence
  Description of Artificial Sequence Synthetic oligonucleotide
caagcagaagacggcatacgagatatctatgctatggcaggttcttaaccgacgtgacagagtgactggagttcagacgtgtgctcttccgatct

SEQ ID NO: 97 - DNA, 95 bp, Artificial Sequence
  Description of Artificial Sequence Synthetic oligonucleotide
caagcagaagacggcatacgagatcggctatggttagaacgtaacgtggtaggtaccgtcagtgactggagttcagacgtgtgctcttccgatct

SEQ ID NO: 98 - DNA, 95 bp, Artificial Sequence
  Description of Artificial Sequence Synthetic oligonucleotide
caagcagaagacggcatacgagatttagcggacaatctcctttaacgcatggttacaagccgtgactggagttcagacgtgtgctcttccgatct

SEQ ID NO: 99 - DNA, 95 bp, Artificial Sequence
  Description of Artificial Sequence Synthetic oligonucleotide
caagcagaagacggcatacgagatccacaattctctaagacggtcgcgtacaagatcgaaggtgactggagttcagacgtgtgctcttccgatct

SEQ ID NO: 100 - DNA, 95 bp, Artificial Sequence
  Description of Artificial Sequence Synthetic oligonucleotide
caagcagaagacggcatacgagatcaaccacgactactgcgttgaactgaggaggttcgttgtgactggagttcagacgtgtgctcttccgatct

SEQ ID NO: 101 - DNA, 99 bp, Artificial Sequence
  Description of Artificial Sequence Synthetic oligonucleotide
aatgatacggcgaccaccgagatctacacacaagcgtccactatagggagcaagaagcttgcagtcacactctttccctacacgacgctcttccgatct

SEQ ID NO: 102 - DNA, 99 bp, Artificial Sequence
  Description of Artificial Sequence Synthetic oligonucleotide
aatgatacggcgaccaccgagatctacactacagtcgcaccgaacatgtatcacacgtgtgtctacacactctttccctacacgacgctcttccgatct

SEQ ID NO: 103 - DNA, 99 bp, Artificial Sequence
  Description of Artificial Sequence Synthetic oligonucleotide
aatgatacggcgaccaccgagatctacacgagtatccacatatagcttctgacggcgagtatggacacactctttccctacacgacgctcttccgatct

SEQ ID NO: 104 - DNA, 99 bp, Artificial Sequence
  Description of Artificial Sequence Synthetic oligonucleotide
aatgatacggcgaccaccgagatctacaccttcaagatgtctgccgtcgcggagttacagagtggaacactctttccctacacgacgctcttccgatct

SEQ ID NO: 105 - DNA, 99 bp, Artificial Sequence
  Description of Artificial Sequence Synthetic oligonucleotide
aatgatacggcgaccaccgagatctacacatcaatacttgtggctgtcaaccacttgacttactggacactctttccctacacgacgctcttccgatct

SEQ ID NO: 106 - DNA, 99 bp, Artificial Sequence
  Description of Artificial Sequence Synthetic oligonucleotide
aatgatacggcgaccaccgagatctacacagtcttgcaagtacgcctgtgtattggactcatgagcacactctttccctacacgacgctcttccgatct

SEQ ID NO: 107 - DNA, 99 bp, Artificial Sequence
  Description of Artificial Sequence Synthetic oligonucleotide
aatgatacggcgaccaccgagatctacactgcctatagaccaccgattgcgcctgactgccatcttacactctttccctacacgacgctcttccgatct

SEQ ID NO: 108 - DNA, 99 bp, Artificial Sequence
  Description of Artificial Sequence Synthetic oligonucleotide
aatgatacggcgaccaccgagatctacacagagatgcggcgtactattctgccacgcttagcttggacactctttccctacacgacgctcttccgatct

SEQ ID NO: 109 - DNA, 99 bp, Artificial Sequence
  Description of Artificial Sequence Synthetic oligonucleotide
aatgatacggcgaccaccgagatctacacattgcattattacgaggttgattaggaggagtgcatcacactctttccctacacgacgctcttccgatct

SEQ ID NO: 110 - DNA, 99 bp, Artificial Sequence
  Description of Artificial Sequence Synthetic oligonucleotide
aatgatacggcgaccaccgagatctacacgtagtagagtacagacaccacaggcattatcgttcgaacactctttccctacacgacgctcttccgatct

SEQ ID NO: 111 - DNA, 99 bp, Artificial Sequence
  Description of Artificial Sequence Synthetic oligonucleotide
aatgatacggcgaccaccgagatctacaccgcaatttccgcattataaccacggaggtggaacaacacactctttccctacacgacgctcttccgatct

SEQ ID NO: 112 - DNA, 99 bp, Artificial Sequence
  Description of Artificial Sequence Synthetic oligonucleotide
aatgatacggcgaccaccgagatctacaccgagctgcatcaggcgttcatgcatgaacacctcgttacactctttccctacacgacgctcttccgatct

SEQ ID NO: 113 - DNA, 99 bp, Artificial Sequence
  Description of Artificial Sequence Synthetic oligonucleotide
aatgatacggcgaccaccgagatctacacgaacggttcaactcgcgttacggtgctatttgcgtgtacactctttccctacacgacgctcttccgatct

SEQ ID NO: 114 - DNA, 99 bp, Artificial Sequence
  Description of Artificial Sequence Synthetic oligonucleotide
aatgatacggcgaccaccgagatctacactgtctccgcaacaacgcttctccattacgagttgccaacactctttccctacacgacgctcttccgatct

SEQ ID NO: 115 - DNA, 99 bp, Artificial Sequence
  Description of Artificial Sequence Synthetic oligonucleotide
aatgatacggcgaccaccgagatctacacttcgccacgttacggtattcgagtcggacgtggtcaaacactctttccctacacgacgctcttccgatct

SEQ ID NO: 116 - DNA, 99 bp, Artificial Sequence
  Description of Artificial Sequence Synthetic oligonucleotide
aatgatacggcgaccaccgagatctacactgtgcgactcgctaatcttagcctggtccttcagacgacactctttccctacacgacgctcttccgatct

SEQ ID NO: 117 - DNA, 99 bp, Artificial Sequence
  Description of Artificial Sequence Synthetic oligonucleotide
aatgatacggcgaccaccgagatctacacaagacgtggactaacgtttactgtatgcggctactagacactctttccctacacgacgctcttccgatct

SEQ ID NO: 118 - DNA, 99 bp, Artificial Sequence
  Description of Artificial Sequence Synthetic oligonucleotide
aatgatacggcgaccaccgagatctacacatggagtcggtaatagtcctctagaatgaattcgtggacactctttccctacacgacgctcttccgatct

SEQ ID NO: 119 - DNA, 99 bp, Artificial Sequence
  Description of Artificial Sequence Synthetic oligonucleotide
aatgatacggcgaccaccgagatctacacaacgaggtcgacgttgtgacgagcagttgcctgattcacactctttccctacacgacgctcttccgatct

SEQ ID NO: 120 - DNA, 99 bp, Artificial Sequence
  Description of Artificial Sequence Synthetic oligonucleotide
aatgatacggcgaccaccgagatctacacagcttgccggacttaggttcctggcttctaatgccagacactctttccctacacgacgctcttccgatct

SEQ ID NO: 121 - DNA, 99 bp, Artificial Sequence
  Description of Artificial Sequence Synthetic oligonucleotide
aatgatacggcgaccaccgagatctacacccgatcggttagtagctttcgcatactgttcatctccacactctttccctacacgacgctcttccgatct

SEQ ID NO: 122 - DNA, 99 bp, Artificial Sequence
  Description of Artificial Sequence Synthetic oligonucleotide
aatgatacggcgaccaccgagatctacactgcatcctcctcaggagttcatgtccatcgaaccgttacactctttccctacacgacgctcttccgatct

SEQ ID NO: 123 - DNA, 99 bp, Artificial Sequence
  Description of Artificial Sequence Synthetic oligonucleotide
aatgatacggcgaccaccgagatctacacttctagctggccataactgaacaggcacccagaatgcacactctttccctacacgacgctcttccgatct

SEQ ID NO: 124 - DNA, 99 bp, Artificial Sequence
  Description of Artificial Sequence Synthetic oligonucleotide
aatgatacggcgaccaccgagatctacaccatgatgcttcaacgtgtgagcgaatacatgtgacctacactctttccctacacgacgctcttccgatct

SEQ ID NO: 125 - DNA, 99 bp, Artificial Sequence
  Description of Artificial Sequence Synthetic oligonucleotide
aatgatacggcgaccaccgagatctacacttagtcagcgttagatttccacggttccagcattgctacactctttccctacacgacgctcttccgatct

SEQ ID NO: 126 - DNA, 99 bp, Artificial Sequence
  Description of Artificial Sequence Synthetic oligonucleotide
aatgatacggcgaccaccgagatctacaccagttgcgattaggtcctcagactcctcgcttcttgcacactctttccctacacgacgctcttccgatct

SEQ ID NO: 127 - DNA, 99 bp, Artificial Sequence
  Description of Artificial Sequence Synthetic oligonucleotide
aatgatacggcgaccaccgagatctacacatcggttgtccatgttatgcgctaagtatgagacatgacactctttccctacacgacgctcttccgatct

SEQ ID NO: 128 - DNA, 99 bp, Artificial Sequence
  Description of Artificial Sequence Synthetic oligonucleotide
aatgatacggcgaccaccgagatctacacagtctcgtccggccaattggagttcttccctggtacaacactctttccctacacgacgctcttccgatct

SEQ ID NO: 129 - DNA, 99 bp, Artificial Sequence
  Description of Artificial Sequence Synthetic oligonucleotide
aatgatacggcgaccaccgagatctacaccaatcgaacagtcacgcttctcttcaggatctcggatacactctttccctacacgacgctcttccgatct

SEQ ID NO: 130 - DNA, 99 bp, Artificial Sequence
  Description of Artificial Sequence Synthetic oligonucleotide
aatgatacggcgaccaccgagatctacacacgtggtgaccgtagacgaggaattccgcacttgaggacactctttccctacacgacgctcttccgatct

SEQ ID NO: 131 - DNA, 99 bp, Artificial Sequence
  Description of Artificial Sequence Synthetic oligonucleotide
aatgatacggcgaccaccgagatctacacagagtctagtccggtgtttccttgtgtaccttgtggtacactctttccctacacgacgctcttccgatct

SEQ ID NO: 132 - DNA, 99 bp, Artificial Sequence
  Description of Artificial Sequence Synthetic oligonucleotide
aatgatacggcgaccaccgagatctacacgtgatgggcgcactagttgccagctaacacagtaaccacactctttccctacacgacgctcttccgatct

SEQ ID NO: 133 - DNA, 99 bp, Artificial Sequence
  Description of Artificial Sequence Synthetic oligonucleotide
aatgatacggcgaccaccgagatctacacgtccaggtggctgaagcttcacctaggtttcgtgcttacactctttccctacacgacgctcttccgatct

SEQ ID NO: 134 - DNA, 99 bp, Artificial Sequence
  Description of Artificial Sequence Synthetic oligonucleotide
aatgatacggcgaccaccgagatctacactctcagcgacacagtgcgcttgatcattcaacgagctacactctttccctacacgacgctcttccgatct

SEQ ID NO: 135 - DNA, 99 bp, Artificial Sequence
  Description of Artificial Sequence Synthetic oligonucleotide
aatgatacggcgaccaccgagatctacaccgtaggtgtattgcctattgttgagatcgactcctgaacactctttccctacacgacgctcttccgatct

SEQ ID NO: 136 - DNA, 99 bp, Artificial Sequence
  Description of Artificial Sequence Synthetic oligonucleotide
aatgatacggcgaccaccgagatctacacttcaggtccacggtaactagctgaagagttctgccatacactctttccctacacgacgctcttccgatct

SEQ ID NO: 137 - DNA, 99 bp, Artificial Sequence
  Description of Artificial Sequence Synthetic oligonucleotide
aatgatacggcgaccaccgagatctacacgaaccgacgtatcgcgattcgaccacaactctccaagacactctttccctacacgacgctcttccgatct

SEQ ID NO: 138 - DNA, 99 bp, Artificial Sequence
  Description of Artificial Sequence Synthetic oligonucleotide
aatgatacggcgaccaccgagatctacacacctgcgcgacatcgactccgaagaatagttgccaacacactctttccctacacgacgctcttccgatct

SEQ ID NO: 139 - DNA, 99 bp, Artificial Sequence
  Description of Artificial Sequence Synthetic oligonucleotide
aatgatacggcgaccaccgagatctacacctcatagtccatatgccctcgtaattgtcatctgcagacactctttccctacacgacgctcttccgatct

SEQ ID NO: 140 - DNA, 99 bp, Artificial Sequence
  Description of Artificial Sequence Synthetic oligonucleotide
aatgatacggcgaccaccgagatctacacaggcctgcgctattaagggcgtccattaacctagctaacactctttccctacacgacgctcttccgatct

SEQ ID NO: 141 - DNA, 99 bp, Artificial Sequence
  Description of Artificial Sequence Synthetic oligonucleotide
aatgatacggcgaccaccgagatctacacacagaactgcattagacttcactcttagccgcaattcacactctttccctacacgacgctcttccgatct

SEQ ID NO: 142 - DNA, 99 bp, Artificial Sequence
  Description of Artificial Sequence Synthetic oligonucleotide
aatgatacggcgaccaccgagatctacaccgttaggagcatgttgtttgccggaagaccgctctaaacactctttccctacacgacgctcttccgatct

SEQ ID NO: 143 - DNA, 99 bp, Artificial Sequence
  Description of Artificial Sequence Synthetic oligonucleotide
aatgatacggcgaccaccgagatctacacgaacacctaatctggctttaatcggtgagggcttctaacactctttccctacacgacgctcttccgatct

SEQ ID NO: 144 - DNA, 99 bp, Artificial Sequence
  Description of Artificial Sequence Synthetic oligonucleotide
aatgatacggcgaccaccgagatctacaccttgcacacaggatggtttccgcaaccaacctcaagaacactctttccctacacgacgctcttccgatct

SEQ ID NO: 145 - DNA, 99 bp, Artificial Sequence
  Description of Artificial Sequence Synthetic oligonucleotide
aatgatacggcgaccaccgagatctacacgatcttggctctgtaggttcggttagctctggacgttacactctttccctacacgacgctcttccgatct

SEQ ID NO: 146 - DNA, 99 bp, Artificial Sequence
  Description of Artificial Sequence Synthetic oligonucleotide
aatgatacggcgaccaccgagatctacacagcacttagttctcaggttcggaacttcaacacgaagacactctttccctacacgacgctcttccgatct

SEQ ID NO: 147 - DNA, 99 bp, Artificial Sequence
  Description of Artificial Sequence Synthetic oligonucleotide
aatgatacggcgaccaccgagatctacacgccactctcagcatagctccaagtctcttacgtactcacactctttccctacacgacgctcttccgatct

SEQ ID NO: 148 - DNA, 99 bp, Artificial Sequence
  Description of Artificial Sequence Synthetic oligonucleotide
aatgatacggcgaccaccgagatctacacggtattgatagagaagcttagtggcgccaccacagatacactctttccctacacgacgctcttccgatct

SEQ ID NO: 149 - DNA, 99 bp, Artificial Sequence
  Description of Artificial Sequence Synthetic oligonucleotide
aatgatacggcgaccaccgagatctacacgtatgcggacctcacctttccaccgaaggcctagagtacactctttccctacacgacgctcttccgatct

SEQ ID NO: 150 - DNA, 99 bp, Artificial Sequence
  Description of Artificial Sequence Synthetic oligonucleotide
aatgatacggcgaccaccgagatctacacacgatgatcgtcgattattgaggctctactccttgacacactctttccctacacgacgctcttccgatct

SEQ ID NO: 151 - DNA, 99 bp, Artificial Sequence
  Description of Artificial Sequence Synthetic oligonucleotide
aatgatacggcgaccaccgagatctacacgttgaacctagtacaagttaggacacagagcaagtctacactctttccctacacgacgctcttccgatct

SEQ ID NO: 152 - DNA, 99 bp, Artificial Sequence
  Description of Artificial Sequence Synthetic oligonucleotide
aatgatacggcgaccaccgagatctacacagcagtctcaaccggtatgcggattcatataagcgtcacactctttccctacacgacgctcttccgatct

SEQ ID NO: 153 - DNA, 99 bp, Artificial Sequence
  Description of Artificial Sequence Synthetic oligonucleotide
aatgatacggcgaccaccgagatctacacgaagcggcgaaccagcaccggatgtaaggaacatccgacactctttccctacacgacgctcttccgatct

SEQ ID NO: 154 - DNA, 99 bp, Artificial Sequence
  Description of Artificial Sequence Synthetic oligonucleotide
aatgatacggcgaccaccgagatctacactcgtcacctccatagaatacgacaaggcgctccagttacactctttccctacacgacgctcttccgatct

SEQ ID NO: 155 - DNA, 99 bp, Artificial Sequence
  Description of Artificial Sequence Synthetic oligonucleotide
aatgatacggcgaccaccgagatctacacatcatctcgctgtacgtttctagaggaaggtgtgaagacactctttccctacacgacgctcttccgatct

SEQ ID NO: 156 - DNA, 99 bp, Artificial Sequence
  Description of Artificial Sequence Synthetic oligonucleotide
aatgatacggcgaccaccgagatctacacccactccgtatcaagcaggcgattgaggttccgaactacactctttccctacacgacgctcttccgatct

SEQ ID NO: 157 - DNA, 99 bp, Artificial Sequence
  Description of Artificial Sequence Synthetic oligonucleotide
aatgatacggcgaccaccgagatctacacgcaagttagcacacgagttcgtacgctaaaccggattacactctttccctacacgacgctcttccgatct

SEQ ID NO: 158 - DNA, 99 bp, Artificial Sequence
  Description of Artificial Sequence Synthetic oligonucleotide
aatgatacggcgaccaccgagatctacacttacttcatgacgaacattcttgtaacggtatggccaacactctttccctacacgacgctcttccgatct

SEQ ID NO: 159 - DNA, 99 bp, Artificial Sequence
  Description of Artificial Sequence Synthetic oligonucleotide
aatgatacggcgaccaccgagatctacacgttaatgatgctcggcatgtaaggtggctgagaaccaacactctttccctacacgacgctcttccgatct

SEQ ID NO: 160 - DNA, 99 bp, Artificial Sequence
  Description of Artificial Sequence Synthetic oligonucleotide
aatgatacggcgaccaccgagatctacacctaacggtcactacggaaaccgctccaactcacacacacactctttccctacacgacgctcttccgatct

SEQ ID NO: 161 - DNA, 99 bp, Artificial Sequence
  Description of Artificial Sequence Synthetic oligonucleotide
aatgatacggcgaccaccgagatctacactacttgggcgataggagtgcggtggattcaccaaggaacactctttccctacacgacgctcttccgatct

SEQ ID NO: 162 - DNA, 99 bp, Artificial Sequence
  Description of Artificial Sequence Synthetic oligonucleotide
aatgatacggcgaccaccgagatctacacgttcactgacatgactagtacaactcgagttcacgctacactctttccctacacgacgctcttccgatct

SEQ ID NO: 163 - DNA, 99 bp, Artificial Sequence
  Description of Artificial Sequence Synthetic oligonucleotide
aatgatacggcgaccaccgagatctacactgagagtcgacatatctttgacacggtgcactctgctacactctttccctacacgacgctcttccgatct

SEQ ID NO: 164 - DNA, 99 bp, Artificial Sequence
  Description of Artificial Sequence Synthetic oligonucleotide
aatgatacggcgaccaccgagatctacactcttcgggtctagtcttttcacgcgtctgctacctacacactctttccctacacgacgctcttccgatct

SEQ ID NO: 165 - DNA, 99 bp, Artificial Sequence
  Description of Artificial Sequence Synthetic oligonucleotide
aatgatacggcgaccaccgagatctacacctagagatgtcctcatattgaatccggttggtattccacactctttccctacacgacgctcttccgatct

SEQ ID NO: 166 - DNA, 99 bp, Artificial Sequence
  Description of Artificial Sequence Synthetic oligonucleotide
aatgatacggcgaccaccgagatctacaccattcgggctggctgatacaggaaggagagaatcctcacactctttccctacacgacgctcttccgatct

SEQ ID NO: 167 - DNA, 99 bp, Artificial Sequence
  Description of Artificial Sequence Synthetic oligonucleotide
aatgatacggcgaccaccgagatctacacctgtattctgtaatctcgcaatcagatcattcggtctacactctttccctacacgacgctcttccgatct

SEQ ID NO: 168 - DNA, 99 bp, Artificial Sequence
  Description of Artificial Sequence Synthetic oligonucleotide
aatgatacggcgaccaccgagatctacactctaccacattaacggctgaggtacttgtagtgaaggacactctttccctacacgacgctcttccgatct

SEQ ID NO: 169 - DNA, 99 bp, Artificial Sequence
  Description of Artificial Sequence Synthetic oligonucleotide
aatgatacggcgaccaccgagatctacactctcgtccgccaataccttacgtcgagtagatcttcgacactctttccctacacgacgctcttccgatct

SEQ ID NO: 170 - DNA, 99 bp, Artificial Sequence
  Description of Artificial Sequence Synthetic oligonucleotide
aatgatacggcgaccaccgagatctacaccttacctacgaacttcattcttcttctcccaggttgaacactctttccctacacgacgctcttccgatct

SEQ ID NO: 171 - DNA, 99 bp, Artificial Sequence
  Description of Artificial Sequence Synthetic oligonucleotide
aatgatacggcgaccaccgagatctacacccacggtgtcgaatcgctgacaacctctcctaccagaacactctttccctacacgacgctcttccgatct

SEQ ID NO: 172 - DNA, 99 bp, Artificial Sequence
  Description of Artificial Sequence Synthetic oligonucleotide
aatgatacggcgaccaccgagatctacactctaccggcaagactctttctctcgaactatcctgtcacactctttccctacacgacgctcttccgatct

SEQ ID NO: 173 - DNA, 99 bp, Artificial Sequence
  Description of Artificial Sequence Synthetic oligonucleotide
aatgatacggcgaccaccgagatctacactgtgcacggcatatccgttctctggccaataactgccacactctttccctacacgacgctcttccgatct

SEQ ID NO: 174 - DNA, 99 bp, Artificial Sequence
  Description of Artificial Sequence Synthetic oligonucleotide
aatgatacggcgaccaccgagatctacactcgtcgttctcacacggttccttaagcctaccgttgaacactctttccctacacgacgctcttccgatct

SEQ ID NO: 175 - DNA, 99 bp, Artificial Sequence
  Description of Artificial Sequence Synthetic oligonucleotide
aatgatacggcgaccaccgagatctacacagctctaggcttacacttgcaacgtattgcatctgacacactctttccctacacgacgctcttccgatct

SEQ ID NO: 176 - DNA, 99 bp, Artificial Sequence
  Description of Artificial Sequence Synthetic oligonucleotide
aatgatacggcgaccaccgagatctacactcgtaattccggtaccttgacacaacaggacgcatagacactctttccctacacgacgctcttccgatct

SEQ ID NO: 177 - DNA, 99 bp, Artificial Sequence
  Description of Artificial Sequence Synthetic oligonucleotide
aatgatacggcgaccaccgagatctacacccgtcttccacacaagattctacagactctggttgtgacactctttccctacacgacgctcttccgatct

SEQ ID NO: 178 - DNA, 99 bp, Artificial Sequence
  Description of Artificial Sequence Synthetic oligonucleotide
aatgatacggcgaccaccgagatctacacatgagatgttatgacgctgctacctgtaatcgaatcgacactctttccctacacgacgctcttccgatct

SEQ ID NO: 179 - DNA, 99 bp, Artificial Sequence
  Description of Artificial Sequence Synthetic oligonucleotide
aatgatacggcgaccaccgagatctacactaggaccggagttgcttccatattcggaattgagctgacactctttccctacacgacgctcttccgatct

SEQ ID NO: 180 - DNA, 99 bp, Artificial Sequence
  Description of Artificial Sequence Synthetic oligonucleotide
aatgatacggcgaccaccgagatctacactcttattcatacgttcattccgccaattccaaggtacacactctttccctacacgacgctcttccgatct

SEQ ID NO: 181 - DNA, 99 bp, Artificial Sequence
  Description of Artificial Sequence Synthetic oligonucleotide
aatgatacggcgaccaccgagatctacacggcttctggttggttcttggataacgatccaaccttgacactctttccctacacgacgctcttccgatct

SEQ ID NO: 182 - DNA, 99 bp, Artificial Sequence
  Description of Artificial Sequence Synthetic oligonucleotide
aatgatacggcgaccaccgagatctacacgagcggtccactgacaagtcggataaccgatacgctcacactctttccctacacgacgctcttccgatct

SEQ ID NO: 183 - DNA, 99 bp, Artificial Sequence
  Description of Artificial Sequence Synthetic oligonucleotide
aatgatacggcgaccaccgagatctacacgccagaacgattgtccgtactaggcggtgggctgtatacactctttccctacacgacgctcttccgatct

SEQ ID NO: 184 - DNA, 99 bp, Artificial Sequence
  Description of Artificial Sequence Synthetic oligonucleotide
aatgatacggcgaccaccgagatctacactagaccaagcctaaccattgcgttatagcataaggcgacactctttccctacacgacgctcttccgatct

SEQ ID NO: 185 - DNA, 99 bp, Artificial Sequence
  Description of Artificial Sequence Synthetic oligonucleotide
aatgatacggcgaccaccgagatctacacgaggagatgctcacatcttctcacctcatttgcaaggacactctttccctacacgacgctcttccgatct

SEQ ID NO: 186 - DNA, 99 bp, Artificial Sequence
  Description of Artificial Sequence Synthetic oligonucleotide
aatgatacggcgaccaccgagatctacacacacctcctctgcgagatggcatattaccgattggtcacactctttccctacacgacgctcttccgatct

SEQ ID NO: 187 - DNA, 99 bp, Artificial Sequence
  Description of Artificial Sequence Synthetic oligonucleotide
aatgatacggcgaccaccgagatctacactggcaatcagtcgctgaggaaggactctgttgccttgacactctttccctacacgacgctcttccgatct

SEQ ID NO: 188 - DNA, 99 bp, Artificial Sequence
  Description of Artificial Sequence Synthetic oligonucleotide
aatgatacggcgaccaccgagatctacactgttgattcaagtgtcataggatgatggcggaagagaacactctttccctacacgacgctcttccgatct

SEQ ID NO: 189 - DNA, 99 bp, Artificial Sequence
  Description of Artificial Sequence Synthetic oligonucleotide
aatgatacggcgaccaccgagatctacacgctgacacctgatagccttacacgtaacccaatcggaacactctttccctacacgacgctcttccgatct

SEQ ID NO: 190 - DNA, 99 bp, Artificial Sequence
  Description of Artificial Sequence Synthetic oligonucleotide
aatgatacggcgaccaccgagatctacacgaggttctgtgttcgtcggagatccaacatgacatccacactctttccctacacgacgctcttccgatct

SEQ ID NO: 191 - DNA, 99 bp, Artificial Sequence
  Description of Artificial Sequence Synthetic oligonucleotide
aatgatacggcgaccaccgagatctacacacctcaggaagttacagtcaagaaccgtgcagtggatacactctttccctacacgacgctcttccgatct

SEQ ID NO: 192 - DNA, 99 bp, Artificial Sequence
  Description of Artificial Sequence Synthetic oligonucleotide
aatgatacggcgaccaccgagatctacacacctgaccacgacaccattggcttcaacgggataggtacactctttccctacacgacgctcttccgatct

SEQ ID NO: 193 - DNA, 99 bp, Artificial Sequence
  Description of Artificial Sequence Synthetic oligonucleotide
aatgatacggcgaccaccgagatctacactaagatcactgcgtccatggagtgtgcatctagtgagacactctttccctacacgacgctcttccgatct

SEQ ID NO: 194 - DNA, 99 bp, Artificial Sequence
  Description of Artificial Sequence Synthetic oligonucleotide
aatgatacggcgaccaccgagatctacaccaagaggggtgcctatctagcagctcttattaggtgcacactctttccctacacgacgctcttccgatct

SEQ ID NO: 195 - DNA, 99 bp, Artificial Sequence
  Description of Artificial Sequence Synthetic oligonucleotide
aatgatacggcgaccaccgagatctacacatcgagaggtgaattcattggaggttagatggcatgaacactctttccctacacgacgctcttccgatct

SEQ ID NO: 196 - DNA, 99 bp, Artificial Sequence
  Description of Artificial Sequence Synthetic oligonucleotide
aatgatacggcgaccaccgagatctacacgacacgttatcagctggttatgagtcacgtgcttagcacactctttccctacacgacgctcttccgatct

SEQ ID NO: 197 - DNA, 19 bp, Artificial Sequence
  Description of Artificial Sequence Synthetic primer
aatatcgtgcagcgaggtg

SEQ ID NO: 198 - DNA, 19 bp, Artificial Sequence
  Description of Artificial Sequence Synthetic primer
ccggcatccaccatattct

SEQ ID NO: 199 - DNA, 19 bp, Artificial Sequence
  Description of Artificial Sequence Synthetic probe
aacggcacgtcttcgactt

SEQ ID NO: 200 - DNA, 20 bp, Artificial Sequence
  Description of Artificial Sequence Synthetic primer
acgctgaggctttcaagaat

SEQ ID NO: 201 - DNA, 20 bp, Artificial Sequence
  Description of Artificial Sequence Synthetic primer
tttgttggcgtaaacagtcg

SEQ ID NO: 202 - DNA, 22 bp, Artificial Sequence
  Description of Artificial Sequence Synthetic probe
ctcagaagaggtttcacaacga

SEQ ID NO: 203 - DNA, 21 bp, Artificial Sequence
  Description of Artificial Sequence Synthetic primer
agataatggcacagccaatca

SEQ ID NO: 204 - DNA, 20 bp, Artificial Sequence
  Description of Artificial Sequence Synthetic primer
tatttacgggcacatcatcg

SEQ ID NO: 205 - DNA, 26 bp, Artificial Sequence
  Description of Artificial Sequence Synthetic probe
cgatgccgatatagttgtagatagtc

SEQ ID NO: 206 - DNA, 20 bp, Artificial Sequence
  Description of Artificial Sequence Synthetic primer
ccgacagatccttgtccaac

SEQ ID NO: 207 - DNA, 19 bp, Artificial Sequence
  Description of Artificial Sequence Synthetic primer
gggtgaaaaacgccaactc

SEQ ID NO: 208 - DNA, 19 bp, Artificial Sequence
  Description of Artificial Sequence Synthetic probe
gttggccgttgagccatac

SEQ ID NO: 209 - DNA, 25 bp, Artificial Sequence
  Description of Artificial Sequence Synthetic primer
caactgcatttaacatatcaacagg

SEQ ID NO: 210 - DNA, 21 bp, Artificial Sequence
  Description of Artificial Sequence Synthetic primer
aaatgcccagaagaaaaacct

SEQ ID NO: 211 - DNA, 24 bp, Artificial Sequence
  Description of Artificial Sequence Synthetic probe
ttctccaactcccattaaatatct

SEQ ID NO: 212 - DNA, 20 bp, Artificial Sequence
  Description of Artificial Sequence Synthetic primer
gatggctcgccactttagaa

SEQ ID NO: 213 - DNA, 21 bp, Artificial Sequence
  Description of Artificial Sequence Synthetic primer
cctaatgtttgcatgccgtta

SEQ ID NO: 214 - DNA, 22 bp, Artificial Sequence
  Description of Artificial Sequence Synthetic probe
tgaaccgcgtcatttattattt

SEQ ID NO: 215 - DNA, 19 bp, Artificial Sequence
  Description of Artificial Sequence Synthetic primer
gcgaaacccagcagaaact

SEQ ID NO: 216 - DNA, 24 bp, Artificial Sequence
  Description of Artificial Sequence Synthetic primer
gagtacgttaccgctatattcacg

SEQ ID NO: 217 - DNA, 19 bp, Artificial Sequence
  Description of Artificial Sequence Synthetic probe
tacctttatggccctgctg

SEQ ID NO: 218 - DNA, 19 bp, Artificial Sequence
  Description of Artificial Sequence Synthetic primer
ggcgattttcggtctgact

SEQ ID NO: 219 - DNA, 18 bp, Artificial Sequence
  Description of Artificial Sequence Synthetic primer
cgtcgcgcgtataatgtg

SEQ ID NO: 220 - DNA, 18 bp, Artificial Sequence
  Description of Artificial Sequence Synthetic probe
cgtggggtgaacgctaac

SEQ ID NO: 221 - DNA, 18 bp, Artificial Sequence
  Description of Artificial Sequence Synthetic primer
tatcgccaaccaggatgc

SEQ ID NO: 222 - DNA, 18 bp, Artificial Sequence
  Description of Artificial Sequence Synthetic primer
ccgccaggtaagcattga

SEQ ID NO: 223 - DNA, 20 bp, Artificial Sequence
  Description of Artificial Sequence Synthetic primer
agcgatgcgtattgttcttg

SEQ ID NO: 224 - DNA, 20 bp, Artificial Sequence
  Description of Artificial Sequence Synthetic primer
atatcccataatcgggcaga

SEQ ID NO: 225 - DNA, 20 bp, Artificial Sequence
  Description of Artificial Sequence Synthetic probe
ccgatgaaatatcgcctgat

SEQ ID NO: 226 - DNA, 37 bp, Artificial Sequence
  Description of Artificial Sequence Synthetic oligonucleotide
gagccatttctgcacaacaaatggagaagatacggag

SEQ ID NO: 227 - DNA, 37 bp, Artificial Sequence
  Description of Artificial Sequence Synthetic oligonucleotide
atgaagcgaacgtatagaggaggaggtatagagtctc

SEQ ID NO: 228 - DNA, 37 bp, Artificial Sequence
  Description of Artificial Sequence Synthetic oligonucleotide
cagacatgtggcgaggtcaagcttatgactccacggt

SEQ ID NO: 229 - DNA, 37 bp, Artificial Sequence
  Description of Artificial Sequence Synthetic oligonucleotide
ttcggtgactgcgaacttaatccggtcatcagtccgg

SEQ ID NO: 230 - DNA, 37 bp, Artificial Sequence
  Description of Artificial Sequence Synthetic oligonucleotide
agccagtagtgtgaggataaagagtgacggctagagc

SEQ ID NO: 231 - DNA, 37 bp, Artificial Sequence
  Description of Artificial Sequence Synthetic oligonucleotide
cactgtacttggcagctctgtgtaacgagaacagatg

SEQ ID NO: 232 - DNA, 37 bp, Artificial Sequence
  Description of Artificial Sequence Synthetic oligonucleotide
cgatcttggcgcttctataatgcgctcaagagaacgg

SEQ ID NO: 233 - DNA, 37 bp, Artificial Sequence
  Description of Artificial Sequence Synthetic oligonucleotide
catcagcttgtccttgacaacggtaagcgaccacgat

SEQ ID NO: 234 - DNA, 37 bp, Artificial Sequence
  Description of Artificial Sequence Synthetic oligonucleotide
gctactaccatgccgaagaacatatcgtcattgctct

SEQ ID NO: 235 - DNA, 37 bp, Artificial Sequence
  Description of Artificial Sequence Synthetic oligonucleotide
tgtacaccggtcgaacagctgcacataagataccgac

SEQ ID NO: 236 - DNA, 37 bp, Artificial Sequence
  Description of Artificial Sequence Synthetic oligonucleotide
gttgagagccggcagcataaagtcactcagttgtcac

SEQ ID NO: 237 - DNA, 37 bp, Artificial Sequence
  Description of Artificial Sequence Synthetic oligonucleotide
actccttgaggacaacacaattacgttggaatcgttc

SEQ ID NO: 238 - DNA, 37 bp, Artificial Sequence
  Description of Artificial Sequence Synthetic oligonucleotide
ctccgtaaagaacggtgttgtgacctgcctgcgaacc

SEQ ID NO: 239 - DNA, 37 bp, Artificial Sequence
  Description of Artificial Sequence Synthetic oligonucleotide
tgcacagatatgtggagcaactacaattgggagaaca

SEQ ID NO: 240 - DNA, 37 bp, Artificial Sequence
  Description of Artificial Sequence Synthetic oligonucleotide
acctcagagaggtcacagttacacaactgacggccat

SEQ ID NO: 241 - DNA, 37 bp, Artificial Sequence
  Description of Artificial Sequence Synthetic oligonucleotide
aggtagagggaacattagcatcaatcatggacgctta

SEQ ID NO: 242 - DNA, 37 bp, Artificial Sequence
  Description of Artificial Sequence Synthetic oligonucleotide
gcgaacatgctagtccagaattagcctaagacagtta

SEQ ID NO: 243 - DNA, 37 bp, Artificial Sequence
  Description of Artificial Sequence Synthetic oligonucleotide
ctaggactatgcggtacctgtacaatgaccgtggcca

SEQ ID NO: 244 - DNA, 37 bp, Artificial Sequence
  Description of Artificial Sequence Synthetic oligonucleotide
atctgacccgcaagctattcgtcggcatctttcgcta

SEQ ID NO: 245 - DNA, 37 bp, Artificial Sequence
  Description of Artificial Sequence Synthetic oligonucleotide
tatggacggctccatattggtgatccaggccgtatgg

SEQ ID NO: 246 - DNA, 37 bp, Artificial Sequence
  Description of Artificial Sequence Synthetic oligonucleotide
ccagagaactgcatggccaacaggtggcatgagacgt

SEQ ID NO: 247 - DNA, 37 bp, Artificial Sequence
  Description of Artificial Sequence Synthetic oligonucleotide
agaaggacgcctgaacttggagccatgtatgtctctg

SEQ ID NO: 248 - DNA, 37 bp, Artificial Sequence
  Description of Artificial Sequence Synthetic oligonucleotide
ttgcggaaatgtcgatgtggtatggtcgagaagctcg

SEQ ID NO: 249 - DNA, 37 bp, Artificial Sequence
  Description of Artificial Sequence Synthetic oligonucleotide
ggtatcctagatggatggtacggtgctatcctcggtt

SEQ ID NO: 250 - DNA, 37 bp, Artificial Sequence
  Description of Artificial Sequence Synthetic oligonucleotide
acatagcgcacatatctccagaatcgcagccgccata

SEQ ID NO: 251 - DNA, 37 bp, Artificial Sequence
  Description of Artificial Sequence Synthetic oligonucleotide
agtagctgcttaactagtggtacttggtcactctctc

SEQ ID NO: 252 - DNA, 37 bp, Artificial Sequence
  Description of Artificial Sequence Synthetic oligonucleotide
gacttctcgaaccaacttgttagagaagctggaccag

SEQ ID NO: 253 - DNA, 37 bp, Artificial Sequence
  Description of Artificial Sequence Synthetic oligonucleotide
tacgcgaaccaacaggtccaaacggtgagcgtgtgac

SEQ ID NO: 254 - DNA, 37 bp, Artificial Sequence
  Description of Artificial Sequence Synthetic oligonucleotide
tgcctcaatccaaggcggaattagacacgataggacc

SEQ ID NO: 255 - DNA, 37 bp, Artificial Sequence
  Description of Artificial Sequence Synthetic oligonucleotide
ctgtgaagcaaggtgcgcaacagatctggagtgaact

SEQ ID NO: 256 - DNA, 37 bp, Artificial Sequence
  Description of Artificial Sequence Synthetic oligonucleotide
aggtggtaccgaagtgctaagctgactacagcaccgt

SEQ ID NO: 257 - DNA, 37 bp, Artificial Sequence
  Description of Artificial Sequence Synthetic oligonucleotide
acgaggaagtctctagcttaactgtcgtatgaatctt

SEQ ID NO: 258 - DNA, 37 bp, Artificial Sequence
  Description of Artificial Sequence Synthetic oligonucleotide
gagaggtaaacaacgctcaaattgcagccgtcacgtc

SEQ ID NO: 259 - DNA, 37 bp, Artificial Sequence
  Description of Artificial Sequence Synthetic oligonucleotide
acaactcggcggacgtacaactcaatccgagtagttg

SEQ ID NO: 260 - DNA, 37 bp, Artificial Sequence
  Description of Artificial Sequence Synthetic oligonucleotide
aatccaccaacctctaccaaagttgtaccactgccgt

SEQ ID NO: 261 - DNA, 37 bp, Artificial Sequence
  Description of Artificial Sequence Synthetic oligonucleotide
tctccatcaggatagtcgaaagttcacggcagaatcg

SEQ ID NO: 262 - DNA, 37 bp, Artificial Sequence
  Description of Artificial Sequence Synthetic oligonucleotide
aaccttcgttgaggcattcaacaggtccgtctacgac

SEQ ID NO: 263 - DNA, 37 bp, Artificial Sequence
  Description of Artificial Sequence Synthetic oligonucleotide
ttaccgcacttggaattcgaggcagttgctcagttac

SEQ ID NO: 264 - DNA, 37 bp, Artificial Sequence
  Description of Artificial Sequence Synthetic oligonucleotide
cgaagtgacattggctacaaagagcattagttggaca

SEQ ID NO: 265 - DNA, 37 bp, Artificial Sequence
  Description of Artificial Sequence Synthetic oligonucleotide
ggtcttcatccgcttgtgaactaatgaaccgtccgaa

SEQ ID NO: 266 - DNA, 37 bp, Artificial Sequence
  Description of Artificial Sequence Synthetic oligonucleotide
tgatgctgtcagatcaggaatagaggcatgcttaacc

SEQ ID NO: 267 - DNA, 37 bp, Artificial Sequence
  Description of Artificial Sequence Synthetic oligonucleotide
cgttaaggcggtctctggtaaagagtcacagtacatt

SEQ ID NO: 268 - DNA, 37 bp, Artificial Sequence
  Description of Artificial Sequence Synthetic oligonucleotide
tgtgagacaggttccatcaaattggtgcatggtaacc

SEQ ID NO: 269 - DNA, 37 bp, Artificial Sequence
  Description of Artificial Sequence Synthetic oligonucleotide
gaaggtgtcacaagagacaactgtggaggttctacga

SEQ ID NO: 270 - DNA, 37 bp, Artificial Sequence
  Description of Artificial Sequence Synthetic oligonucleotide
tagtgcagccaacctgagaaaacgaggtgttccatgc

SEQ ID NO: 271 - DNA, 37 bp, Artificial Sequence
  Description of Artificial Sequence Synthetic oligonucleotide
ttcctagccggaacaccgaagagcgctatacgatctt

SEQ ID NO: 272 - DNA, 37 bp, Artificial Sequence
  Description of Artificial Sequence Synthetic oligonucleotide
ggcaacaaccgcttacgcttacctgttaggaatccgt

SEQ ID NO: 273 - DNA, 37 bp, Artificial Sequence
  Description of Artificial Sequence Synthetic oligonucleotide
atatcggctgataacgacggtccaacgccatatggat

SEQ ID NO: 274 - DNA, 37 bp, Artificial Sequence
  Description of Artificial Sequence Synthetic oligonucleotide
taagtcggacctgttcggaaaatgaagcgaatggtta

SEQ ID NO: 275 - DNA, 37 bp, Artificial Sequence
  Description of Artificial Sequence Synthetic oligonucleotide
gaacggaaaaggcgttcgtaaacataggcaaacactc

SEQ ID NO: 276 - DNA, 37 bp, Artificial Sequence
  Description of Artificial Sequence Synthetic oligonucleotide
ttctccgatgtcaaccagaacgaagaacgggttagta

SEQ ID NO: 277 - DNA, 37 bp, Artificial Sequence
  Description of Artificial Sequence Synthetic oligonucleotide
ttggttggttcgatcctcgggcaatcactattgtata

SEQ ID NO: 278 - DNA, 37 bp, Artificial Sequence
  Description of Artificial Sequence Synthetic oligonucleotide
tcgatcctgacagcggagaaattcgcctccagccaac

SEQ ID NO: 279 - DNA, 37 bp, Artificial Sequence
  Description of Artificial Sequence Synthetic oligonucleotide
accatggagtggtattgcaatgtgatgaactccaaga

SEQ ID NO: 280 - DNA, 37 bp, Artificial Sequence
  Description of Artificial Sequence Synthetic oligonucleotide
gcgttcaagtcgccatggtagtgatcgctcgttagtc

SEQ ID NO: 281 - DNA, 37 bp, Artificial Sequence
  Description of Artificial Sequence Synthetic oligonucleotide
gcatggtaaataccagaccaggtcaactggacagacg

SEQ ID NO: 282 - DNA, 37 bp, Artificial Sequence
  Description of Artificial Sequence Synthetic oligonucleotide
accgtacttgcgttgagttatggatagtagtcacagg

SEQ ID NO: 283 - DNA, 37 bp, Artificial Sequence
  Description of Artificial Sequence Synthetic oligonucleotide
gaggtaacccgccaattctgtctcgtgccattggtct

SEQ ID NO: 284 - DNA, 37 bp, Artificial Sequence
  Description of Artificial Sequence Synthetic oligonucleotide
cctctctagataccttcgaaagaagacaagacttcac

SEQ ID NO: 285 - DNA, 37 bp, Artificial Sequence
  Description of Artificial Sequence Synthetic oligonucleotide
cacggatttacggagtggaagctctaccagttgcatc

SEQ ID NO: 286 - DNA, 37 bp, Artificial Sequence
  Description of Artificial Sequence Synthetic oligonucleotide
agtcatccgtgaatgtcgtaggttcagttgcctcaat

SEQ ID NO: 287 - DNA, 37 bp, Artificial Sequence
  Description of Artificial Sequence Synthetic oligonucleotide
gaaccacaatctaggtcttgctcacttaaggacacca

SEQ ID NO: 288 - DNA, 37 bp, Artificial Sequence
  Description of Artificial Sequence Synthetic oligonucleotide
acgcatctcttagtcaccaatgtgtgcatatcaactg

SEQ ID NO: 289 - DNA, 37 bp, Artificial Sequence
  Description of Artificial Sequence Synthetic oligonucleotide
tgtcttggtcgactagccaagccagtaccaatgtctg

SEQ ID NO: 290 - DNA, 37 bp, Artificial Sequence
  Description of Artificial Sequence Synthetic oligonucleotide
atggtaccccacgactgcaattcatccgccattaagc

SEQ ID NO: 291 - DNA, 37 bp, Artificial Sequence
  Description of Artificial Sequence Synthetic oligonucleotide
ctgtccaattctggcggtgagcggcaatgatcctagg

SEQ ID NO: 292 - DNA, 37 bp, Artificial Sequence
  Description of Artificial Sequence Synthetic oligonucleotide
ctcgagttggctggtgacaataagtaggagcaccgtc

SEQ ID NO: 293 - DNA, 37 bp, Artificial Sequence
  Description of Artificial Sequence Synthetic oligonucleotide
ttctcgctcataatacacgggtataagacgggcactg

SEQ ID NO: 294 - DNA, 37 bp, Artificial Sequence
  Description of Artificial Sequence Synthetic oligonucleotide
ctaaccgatgcttctgcgaacctctccgatagcgatt

SEQ ID NO: 295 - DNA, 37 bp, Artificial Sequence
  Description of Artificial Sequence Synthetic oligonucleotide
acggacaactctaggaagaagcattggcctcgactga

SEQ ID NO: 296 - DNA, 37 bp, Artificial Sequence
  Description of Artificial Sequence Synthetic oligonucleotide
cgtacagtgccagtaggtaagaggttgaccttcacgt

SEQ ID NO: 297 - DNA, 37 bp, Artificial Sequence
  Description of Artificial Sequence Synthetic oligonucleotide
tgatcacgggaggccgataatgagcacctcatggctt

SEQ ID NO: 298 - DNA, 37 bp, Artificial Sequence
  Description of Artificial Sequence Synthetic oligonucleotide
cttgactggtagcttgttggagcgaagtcactaacac

SEQ ID NO: 299 - DNA, 37 bp, Artificial Sequence
  Description of Artificial Sequence Synthetic oligonucleotide
ggtgttagttcttaagtgccgatgaatccgggcagtc

SEQ ID NO: 300 - DNA, 37 bp, Artificial Sequence
  Description of Artificial Sequence Synthetic oligonucleotide
aaccgctaatatgctgccaagtattctccagatgcag

SEQ ID NO: 301 - DNA, 37 bp, Artificial Sequence
  Description of Artificial Sequence Synthetic oligonucleotide
acaagaccactgccattgaagacaagcggtggtgtta

SEQ ID NO: 302 - DNA, 37 bp, Artificial Sequence
  Description of Artificial Sequence Synthetic oligonucleotide
gtccgaatgaacgtccttaagacagcttacaatgcca

SEQ ID NO: 303 - DNA, 37 bp, Artificial Sequence
  Description of Artificial Sequence Synthetic oligonucleotide
acagttccaaggaacgagaacattgcgcgacacaccg

SEQ ID NO: 304 - DNA, 37 bp, Artificial Sequence
  Description of Artificial Sequence Synthetic oligonucleotide
agagacagcaacaatcggaatatgctacgttcctaca

SEQ ID NO: 305 - DNA, 37 bp, Artificial Sequence
  Description of Artificial Sequence Synthetic oligonucleotide
gccgataaacttcggcctcaagctgcactgtcagtga

SEQ ID NO: 306 - DNA, 37 bp, Artificial Sequence
  Description of Artificial Sequence Synthetic oligonucleotide
tggagcaacctcggatacgtatacgtacctccagtgt

SEQ ID NO: 307 - DNA, 37 bp, Artificial Sequence
  Description of Artificial Sequence Synthetic oligonucleotide
cagtggaaactagaagctggtgtaggccactagagag

SEQ ID NO: 308 - DNA, 37 bp, Artificial Sequence
  Description of Artificial Sequence Synthetic oligonucleotide
aatgcctgggcaatcttgaacttcgcacgtataagtc

SEQ ID NO: 309 - DNA, 37 bp, Artificial Sequence
  Description of Artificial Sequence Synthetic oligonucleotide
agtgtcgtaattgtaaccgggccatagaacggcgtga

SEQ ID NO: 310 - DNA, 37 bp, Artificial Sequence
  Description of Artificial Sequence Synthetic oligonucleotide
ccacttgacggtgttatgaagcgctgtcattccacct

SEQ ID NO: 311 - DNA, 37 bp, Artificial Sequence
  Description of Artificial Sequence Synthetic oligonucleotide
agtgccaacgacttcactgaatgcagttcatgctcaa

SEQ ID NO: 312 - DNA, 37 bp, Artificial Sequence
  Description of Artificial Sequence Synthetic oligonucleotide
ggtgcataaggagtgtctccgaccatacggctgattg

SEQ ID NO: 313 - DNA, 37 bp, Artificial Sequence
  Description of Artificial Sequence Synthetic oligonucleotide
caatctccatgttggccgtaaagcaagcatgttgata

SEQ ID NO: 314 - DNA, 37 bp, Artificial Sequence
  Description of Artificial Sequence Synthetic oligonucleotide
gttatcggaagttgctctgattaagcaacgctccata

SEQ ID NO: 315 - DNA, 37 bp, Artificial Sequence
  Description of Artificial Sequence Synthetic oligonucleotide
ggaacttccgagtataggcagagtgcgaactaatgca

SEQ ID NO: 316 - DNA, 37 bp, Artificial Sequence
  Description of Artificial Sequence Synthetic oligonucleotide
tctgaagcatcggaaggcaatggctcctatccattca

SEQ ID NO: 317 - DNA, 37 bp, Artificial Sequence
  Description of Artificial Sequence Synthetic oligonucleotide
tctgtcacgtcggttaagaacctgccatagcatagat

SEQ ID NO: 318 - DNA, 37 bp, Artificial Sequence
  Description of Artificial Sequence Synthetic oligonucleotide
tgacggtacctaccacgttacgttctaaccatagccg

SEQ ID NO: 319 - DNA, 37 bp, Artificial Sequence
  Description of Artificial Sequence Synthetic oligonucleotide
ggcttgtaaccatgcgttaaaggagattgtccgctaa

SEQ ID NO: 320 - DNA, 37 bp, Artificial Sequence
  Description of Artificial Sequence Synthetic oligonucleotide
cttcgatcttgtacgcgaccgtcttagagaattgtgg

SEQ ID NO: 321 - DNA, 37 bp, Artificial Sequence
  Description of Artificial Sequence Synthetic oligonucleotide
aacgaacctcctcagttcaacgcagtagtcgtggttg

SEQ ID NO: 322 - DNA, 37 bp, Artificial Sequence
  Description of Artificial Sequence Synthetic oligonucleotide
gactgcaagcttcttgctccctatagtggacgcttgt

SEQ ID NO: 323 - DNA, 37 bp, Artificial Sequence
  Description of Artificial Sequence Synthetic oligonucleotide
gtagacacacgtgtgatacatgttcggtgcgactgta

SEQ ID NO: 324 - DNA, 37 bp, Artificial Sequence
  Description of Artificial Sequence Synthetic oligonucleotide
gtccatactcgccgtcagaagctatatgtggatactc

SEQ ID NO: 325 - DNA, 37 bp, Artificial Sequence
  Description of Artificial Sequence Synthetic oligonucleotide
tccactctgtaactccgcgacggcagacatcttgaag

SEQ ID NO: 326 - DNA, 37 bp, Artificial Sequence
  Description of Artificial Sequence Synthetic oligonucleotide
ccagtaagtcaagtggttgacagccacaagtattgat

SEQ ID NO: 327 - DNA, 37 bp, Artificial Sequence
  Description of Artificial Sequence Synthetic oligonucleotide
gctcatgagtccaatacacaggcgtacttgcaagact

SEQ ID NO: 328 - DNA, 37 bp, Artificial Sequence
  Description of Artificial Sequence Synthetic oligonucleotide
aagatggcagtcaggcgcaatcggtggtctataggca

SEQ ID NO: 329 - DNA, 37 bp, Artificial Sequence
  Description of Artificial Sequence Synthetic oligonucleotide
ccaagctaagcgtggcagaatagtacgccgcatctct

SEQ ID NO: 330 - DNA, 37 bp, Artificial Sequence
  Description of Artificial Sequence Synthetic oligonucleotide
gatgcactcctcctaatcaacctcgtaataatgcaat

SEQ ID NO: 331 - DNA, 37 bp, Artificial Sequence
  Description of Artificial Sequence Synthetic oligonucleotide
tcgaacgataatgcctgtggtgtctgtactctactac

SEQ ID NO: 332 - DNA, 37 bp, Artificial Sequence
  Description of Artificial Sequence Synthetic oligonucleotide
gttgttccacctccgtggttataatgcggaaattgcg

SEQ ID NO: 333 - DNA, 37 bp, Artificial Sequence
  Description of Artificial Sequence Synthetic oligonucleotide
aacgaggtgttcatgcatgaacgcctgatgcagctcg

SEQ ID NO: 334 - DNA, 37 bp, Artificial Sequence
  Description of Artificial Sequence Synthetic oligonucleotide
acacgcaaatagcaccgtaacgcgagttgaaccgttc

SEQ ID NO: 335 - DNA, 37 bp, Artificial Sequence
  Description of Artificial Sequence Synthetic oligonucleotide
tggcaactcgtaatggagaagcgttgttgcggagaca

SEQ ID NO: 336 - DNA, 37 bp, Artificial Sequence
  Description of Artificial Sequence Synthetic oligonucleotide
ttgaccacgtccgactcgaataccgtaacgtggcgaa

SEQ ID NO: 337 - DNA, 37 bp, Artificial Sequence
  Description of Artificial Sequence Synthetic oligonucleotide
cgtctgaaggaccaggctaagattagcgagtcgcaca

SEQ ID NO: 338 - DNA, 37 bp, Artificial Sequence
  Description of Artificial Sequence Synthetic oligonucleotide
ctagtagccgcatacagtaaacgttagtccacgtctt

SEQ ID NO: 339 - DNA, 37 bp, Artificial Sequence
  Description of Artificial Sequence Synthetic oligonucleotide
ccacgaattcattctagaggactattaccgactccat

SEQ ID NO: 340 - DNA, 37 bp, Artificial Sequence
  Description of Artificial Sequence Synthetic oligonucleotide
gaatcaggcaactgctcgtcacaacgtcgacctcgtt

SEQ ID NO: 341 - DNA, 37 bp, Artificial Sequence
  Description of Artificial Sequence Synthetic oligonucleotide
ctggcattagaagccaggaacctaagtccggcaagct

SEQ ID NO: 342 - DNA, 37 bp, Artificial Sequence
  Description of Artificial Sequence Synthetic oligonucleotide
ggagatgaacagtatgcgaaagctactaaccgatcgg

SEQ ID NO: 343 - DNA, 37 bp, Artificial Sequence
  Description of Artificial Sequence Synthetic oligonucleotide
aacggttcgatggacatgaactcctgaggaggatgca

SEQ ID NO: 344 - DNA, 37 bp, Artificial Sequence
  Description of Artificial Sequence Synthetic oligonucleotide
gcattctgggtgcctgttcagttatggccagctagaa

SEQ ID NO: 345 - DNA, 37 bp, Artificial Sequence
  Description of Artificial Sequence Synthetic oligonucleotide
aggtcacatgtattcgctcacacgttgaagcatcatg

SEQ ID NO: 346 - DNA, 37 bp, Artificial Sequence
  Description of Artificial Sequence Synthetic oligonucleotide
agcaatgctggaaccgtggaaatctaacgctgactaa

SEQ ID NO: 347 - DNA, 37 bp, Artificial Sequence
  Description of Artificial Sequence Synthetic oligonucleotide
gcaagaagcgaggagtctgaggacctaatcgcaactg

SEQ ID NO: 348 - DNA, 37 bp, Artificial Sequence
  Description of Artificial Sequence Synthetic oligonucleotide
catgtctcatacttagcgcataacatggacaaccgat

SEQ ID NO: 349 - DNA, 37 bp, Artificial Sequence
  Description of Artificial Sequence Synthetic oligonucleotide
tgtaccagggaagaactccaattggccggacgagact

SEQ ID NO: 350 - DNA, 37 bp, Artificial Sequence
  Description of Artificial Sequence Synthetic oligonucleotide
atccgagatcctgaagagaagcgtgactgttcgattg

SEQ ID NO: 351 - DNA, 37 bp, Artificial Sequence
  Description of Artificial Sequence Synthetic oligonucleotide
cctcaagtgcggaattcctcgtctacggtcaccacgt

SEQ ID NO: 352 - DNA, 37 bp, Artificial Sequence
  Description of Artificial Sequence Synthetic oligonucleotide
accacaaggtacacaaggaaacaccggactagactct

SEQ ID NO: 353 - DNA, 37 bp, Artificial Sequence
  Description of Artificial Sequence Synthetic oligonucleotide
ggttactgtgttagctggcaactagtgcgcccatcac

SEQ ID NO: 354 - DNA, 37 bp, Artificial Sequence
  Description of Artificial Sequence Synthetic oligonucleotide
aagcacgaaacctaggtgaagcttcagccacctggac

SEQ ID NO: 355 - DNA, 37 bp, Artificial Sequence
  Description of Artificial Sequence Synthetic oligonucleotide
agctcgttgaatgatcaagcgcactgtgtcgctgaga

SEQ ID NO: 356 - DNA, 37 bp, Artificial Sequence
  Description of Artificial Sequence Synthetic oligonucleotide
tcaggagtcgatctcaacaataggcaatacacctacg

SEQ ID NO: 357 - DNA, 37 bp, Artificial Sequence
  Description of Artificial Sequence Synthetic oligonucleotide
atggcagaactcttcagctagttaccgtggacctgaa

SEQ ID NO: 358 - DNA, 37 bp, Artificial Sequence
  Description of Artificial Sequence Synthetic oligonucleotide
cttggagagttgtggtcgaatcgcgatacgtcggttc

SEQ ID NO: 359 - DNA, 37 bp, Artificial Sequence
  Description of Artificial Sequence Synthetic oligonucleotide
gttggcaactattcttcggagtcgatgtcgcgcaggt

SEQ ID NO: 360 - DNA, 37 bp, Artificial Sequence
  Description of Artificial Sequence Synthetic oligonucleotide
ctgcagatgacaattacgagggcatatggactatgag

SEQ ID NO: 361 - DNA, 37 bp, Artificial Sequence
  Description of Artificial Sequence Synthetic oligonucleotide
tagctaggttaatggacgcccttaatagcgcaggcct

SEQ ID NO: 362 - DNA, 37 bp, Artificial Sequence
  Description of Artificial Sequence Synthetic oligonucleotide
gaattgcggctaagagtgaagtctaatgcagttctgt

SEQ ID NO: 363 - DNA, 37 bp, Artificial Sequence
  Description of Artificial Sequence Synthetic oligonucleotide
ttagagcggtcttccggcaaacaacatgctcctaacg

SEQ ID NO: 364 - DNA, 37 bp, Artificial Sequence
  Description of Artificial Sequence Synthetic oligonucleotide
tagaagccctcaccgattaaagccagattaggtgttc

SEQ ID NO: 365 - DNA, 37 bp, Artificial Sequence
  Description of Artificial Sequence Synthetic oligonucleotide
tcttgaggttggttgcggaaaccatcctgtgtgcaag

SEQ ID NO: 366 - DNA, 37 bp, Artificial Sequence
  Description of Artificial Sequence Synthetic oligonucleotide
aacgtccagagctaaccgaacctacagagccaagatc

SEQ ID NO: 367 - DNA, 37 bp, Artificial Sequence
  Description of Artificial Sequence Synthetic oligonucleotide
cttcgtgttgaagttccgaacctgagaactaagtgct

SEQ ID NO: 368 - DNA, 37 bp, Artificial Sequence
  Description of Artificial Sequence Synthetic oligonucleotide
gagtacgtaagagacttggagctatgctgagagtggc

SEQ ID NO: 369 - DNA, 37 bp, Artificial Sequence
  Description of Artificial Sequence Synthetic oligonucleotide
atctgtggtggcgccactaagcttctctatcaatacc

SEQ ID NO: 370 - DNA, 37 bp, Artificial Sequence
  Description of Artificial Sequence Synthetic oligonucleotide
actctaggccttcggtggaaaggtgaggtccgcatac

SEQ ID NO: 371 - DNA, 37 bp, Artificial Sequence
  Description of Artificial Sequence Synthetic oligonucleotide
gtcaaggagtagagcctcaataatcgacgatcatcgt

SEQ ID NO: 372 - DNA, 37 bp, Artificial Sequence
  Description of Artificial Sequence Synthetic oligonucleotide
agacttgctctgtgtcctaacttgtactaggttcaac

SEQ ID NO: 373 - DNA, 37 bp, Artificial Sequence
  Description of Artificial Sequence Synthetic oligonucleotide
gacgcttatatgaatccgcataccggttgagactgct

SEQ ID NO: 374 - DNA, 37 bp, Artificial Sequence
  Description of Artificial Sequence Synthetic oligonucleotide
cggatgttccttacatccggtgctggttcgccgcttc

SEQ ID NO: 375 - DNA, 37 bp, Artificial Sequence
  Description of Artificial Sequence Synthetic oligonucleotide
aactggagcgccttgtcgtattctatggaggtgacga

SEQ ID NO: 376 - DNA, 37 bp, Artificial Sequence
  Description of Artificial Sequence Synthetic oligonucleotide
cttcacaccttcctctagaaacgtacagcgagatgat

SEQ ID NO: 377 - DNA, 37 bp, Artificial Sequence
  Description of Artificial Sequence Synthetic oligonucleotide
agttcggaacctcaatcgcctgcttgatacggagtgg

SEQ ID NO: 378 - DNA, 37 bp, Artificial Sequence
  Description of Artificial Sequence Synthetic oligonucleotide
aatccggtttagcgtacgaactcgtgtgctaacttgc

SEQ ID NO: 379 - DNA, 37 bp, Artificial Sequence
  Description of Artificial Sequence Synthetic oligonucleotide
tggccataccgttacaagaatgttcgtcatgaagtaa

SEQ ID NO: 380 - DNA, 37 bp, Artificial Sequence
  Description of Artificial Sequence Synthetic oligonucleotide
tggttctcagccaccttacatgccgagcatcattaac

SEQ ID NO: 381 - DNA, 37 bp, Artificial Sequence
  Description of Artificial Sequence Synthetic oligonucleotide
gtgtgtgagttggagcggtttccgtagtgaccgttag

SEQ ID NO: 382 - DNA, 37 bp, Artificial Sequence
  Description of Artificial Sequence Synthetic oligonucleotide
tccttggtgaatccaccgcactcctatcgcccaagta

SEQ ID NO: 383 - DNA, 37 bp, Artificial Sequence
  Description of Artificial Sequence Synthetic oligonucleotide
agcgtgaactcgagttgtactagtcatgtcagtgaac

SEQ ID NO: 384 - DNA, 37 bp, Artificial Sequence
  Description of Artificial Sequence Synthetic oligonucleotide
agcagagtgcaccgtgtcaaagatatgtcgactctca

SEQ ID NO: 385 - DNA, 37 bp, Artificial Sequence
  Description of Artificial Sequence Synthetic oligonucleotide
gtaggtagcagacgcgtgaaaagactagacccgaaga

SEQ ID NO: 386 - DNA, 37 bp, Artificial Sequence
  Description of Artificial Sequence Synthetic oligonucleotide
ggaataccaaccggattcaatatgaggacatctctag

SEQ ID NO: 387 - DNA, 37 bp, Artificial Sequence
  Description of Artificial Sequence Synthetic oligonucleotide
gaggattctctccttcctgtatcagccagcccgaatg

SEQ ID NO: 388 - DNA, 37 bp, Artificial Sequence
  Description of Artificial Sequence Synthetic oligonucleotide
agaccgaatgatctgattgcgagattacagaatacag

SEQ ID NO: 389 - DNA, 37 bp, Artificial Sequence
  Description of Artificial Sequence Synthetic oligonucleotide
ccttcactacaagtacctcagccgttaatgtggtaga

SEQ ID NO: 390 - DNA, 37 bp, Artificial Sequence
  Description of Artificial Sequence Synthetic oligonucleotide
cgaagatctactcgacgtaaggtattggcggacgaga

SEQ ID NO: 391 - DNA, 37 bp, Artificial Sequence
  Description of Artificial Sequence Synthetic oligonucleotide
tcaacctgggagaagaagaatgaagttcgtaggtaag

SEQ ID NO: 392 - DNA, 37 bp, Artificial Sequence
  Description of Artificial Sequence Synthetic oligonucleotide
tctggtaggagaggttgtcagcgattcgacaccgtgg

SEQ ID NO: 393 - DNA, 37 bp, Artificial Sequence
  Description of Artificial Sequence Synthetic oligonucleotide
gacaggatagttcgagagaaagagtcttgccggtaga

SEQ ID NO: 394 - DNA, 37 bp, Artificial Sequence
  Description of Artificial Sequence Synthetic oligonucleotide
ggcagttattggccagagaacggatatgccgtgcaca

SEQ ID NO: 395 - DNA, 37 bp, Artificial Sequence
  Description of Artificial Sequence Synthetic oligonucleotide
tcaacggtaggcttaaggaaccgtgtgagaacgacga

SEQ ID NO: 396 - DNA, 37 bp, Artificial Sequence
  Description of Artificial Sequence Synthetic oligonucleotide
gtcagatgcaatacgttgcaagtgtaagcctagagct

SEQ ID NO: 397 - DNA, 37 bp, Artificial Sequence
  Description of Artificial Sequence Synthetic oligonucleotide
ctatgcgtcctgttgtgtcaaggtaccggaattacga

SEQ ID NO: 398 - DNA, 37 bp, Artificial Sequence
  Description of Artificial Sequence Synthetic oligonucleotide
cacaaccagagtctgtagaatcttgtgtggaagacgg

SEQ ID NO: 399 - DNA, 37 bp, Artificial Sequence
  Description of Artificial Sequence Synthetic oligonucleotide
cgattcgattacaggtagcagcgtcataacatctcat

SEQ ID NO: 400 - DNA, 37 bp, Artificial Sequence
  Description of Artificial Sequence Synthetic oligonucleotide
cagctcaattccgaatatggaagcaactccggtccta

SEQ ID NO: 401 - DNA, 37 bp, Artificial Sequence
  Description of Artificial Sequence Synthetic oligonucleotide
gtaccttggaattggcggaatgaacgtatgaataaga

SEQ ID NO: 402 - DNA, 37 bp, Artificial Sequence
  Description of Artificial Sequence Synthetic oligonucleotide
caaggttggatcgttatccaagaaccaaccagaagcc

SEQ ID NO: 403 - DNA, 37 bp, Artificial Sequence
  Description of Artificial Sequence Synthetic oligonucleotide
gagcgtatcggttatccgacttgtcagtggaccgctc

SEQ ID NO: 404 - DNA, 37 bp, Artificial Sequence
  Description of Artificial Sequence Synthetic oligonucleotide
atacagcccaccgcctagtacggacaatcgttctggc

SEQ ID NO: 405 - DNA, 37 bp, Artificial Sequence
  Description of Artificial Sequence Synthetic oligonucleotide
cgccttatgctataacgcaatggttaggcttggtcta

SEQ ID NO: 406 - DNA, 37 bp, Artificial Sequence
  Description of Artificial Sequence Synthetic oligonucleotide
ccttgcaaatgaggtgagaagatgtgagcatctcctc

SEQ ID NO: 407 - DNA, 37 bp, Artificial Sequence
  Description of Artificial Sequence Synthetic oligonucleotide
gaccaatcggtaatatgccatctcgcagaggaggtgt

SEQ ID NO: 408 - DNA, 37 bp, Artificial Sequence
  Description of Artificial Sequence Synthetic oligonucleotide
caaggcaacagagtccttcctcagcgactgattgcca

SEQ ID NO: 409 - DNA, 37 bp, Artificial Sequence
  Description of Artificial Sequence Synthetic oligonucleotide
tctcttccgccatcatcctatgacacttgaatcaaca

SEQ ID NO: 410 - DNA, 37 bp, Artificial Sequence
  Description of Artificial Sequence Synthetic oligonucleotide
tccgattgggttacgtgtaaggctatcaggtgtcagc

SEQ ID NO: 411 - DNA, 37 bp, Artificial Sequence
  Description of Artificial Sequence Synthetic oligonucleotide
ggatgtcatgttggatctccgacgaacacagaacctc

SEQ ID NO: 412 - DNA, 37 bp, Artificial Sequence
  Description of Artificial Sequence Synthetic oligonucleotide
atccactgcacggttcttgactgtaacttcctgaggt

SEQ ID NO: 413 - DNA, 37 bp, Artificial Sequence
  Description of Artificial Sequence Synthetic oligonucleotide
acctatcccgttgaagccaatggtgtcgtggtcaggt

SEQ ID NO: 414 - DNA, 37 bp, Artificial Sequence
  Description of Artificial Sequence Synthetic oligonucleotide
ctcactagatgcacactccatggacgcagtgatctta

SEQ ID NO: 415 - DNA, 37 bp, Artificial Sequence
  Description of Artificial Sequence Synthetic oligonucleotide
gcacctaataagagctgctagataggcacccctcttg

SEQ ID NO: 416 - DNA, 37 bp, Artificial Sequence
  Description of Artificial Sequence Synthetic oligonucleotide
tcatgccatctaacctccaatgaattcacctctcgat

SEQ ID NO: 417 - DNA, 37 bp, Artificial Sequence
  Description of Artificial Sequence Synthetic oligonucleotide
gctaagcacgtgactcataaccagctgataacgtgtc

SEQ ID NO: 418 - DNA, 83 bp, Unknown
  Description of Unknown Klebsiella pneumoniae sequence
aattcgcccttatcgccaaccaggatgccttaaccggaaccgctaaccgtcgggcgctcaatgcttacctggcggaagggcga

SEQ ID NO: 419 - DNA, 83 bp, Artificial Sequence; identical to SEQ ID NO: 418
  Description of Artificial Sequence Synthetic oligonucleotide